SEQUENCE LISTING (252 sequences)

SEQ ID NO: 1 - DNA, 317 bp, Rhizoctonia solani ZG3
gcggcggatcaccgtggcggccgcatccaagattcttgccaacttgacttatgcttacaagggctacggccttagtatgggaacaatgctgtgtggtgtgactccccaagaaggacctgctctatactacattgattcagacggcacccgtttacccggtaacctgttttgtgttggatccggtcagacattcgcgtacggtgtgctggactctaactaccgctacgatttgagcgaggaggaagctcttgctctgggccgtcgtgcgatcttggccgctatgcaccgtgatgcatactgcggcggcttcatcaacc

SEQ ID NO: 2 - protein, 105 aa, Rhizoctonia solani ZG3
RRITVAAASKILANLTYAYKGYGLSMGTMLCGVTPQEGPALYYIDSDGTRLPGNLFCVGSGQTFAYGVLDSNYRYDLSEEEALALGRRAILAAMHRDAYCGGFIN

SEQ ID NO: 3 - DNA, 291 bp, Rhizoctonia solani ZG5
caagattccttgccaacttgacttatgcttacaagggctacggccttagtatgggaacaatgctgtgtggtgtgactccccaagaaggacctgctctatactacattgattcagacggcacccgtttacccggtaacctgttttgtgttggatccggtcagacattcgcgtacggtgtgctggactctaactaccgctacgatttgagcgaggaggaagctcttgctctgggccgtcgtgcgatcttggccgctatgcaccgtgatgcttactctggtggttacatcaaag

SEQ ID NO: 4 - protein, 96 aa, Rhizoctonia solani ZG5
RFLANLTYAYKGYGLSMGTMLCGVTPQEGPALYYIDSDGTRLPGNLFCVGSGQTFAYGVLDSNYRYDLSEEEALALGRRAILAAMHRDAYSGGYIK

SEQ ID NO: 5 - DNA, 291 bp, Rhizoctonia solani rice infecting strain
caagattccttgccaacttgacttatgcttacaagggctacagccttagtatgggaacaatgctgtgtggtgtgactccccaagaaggacctgctctatactacattgattcagacggcacccgtttacccggtaacctgttttgtgttggatccggtcagacattcgcgtacggtgtgctggactctaactaccgctacgatttgagcgaggaggaagctcttgctctgggccgtcgtgcgatcttggccgctatgcaccgtgatgcttactctggtggttacatcaaag

SEQ ID NO: 6 - protein, 96 aa, Rhizoctonia solani rice infecting strain
RFLANLTYAYKGYSLSMGTMLCGVTPQEGPALYYIDSDGTRLPGNLFCVGSGQTFAYGVLDSNYRYDLSEEEALALGRRAILAAMHRDAYSGGYIK

SEQ ID NO: 7 - DNA, 300 bp, Rhizoctonia solani ZG3
cgtggcggccgcatccaagattcttgccaacttgacttatgcttacaagggctacggccttagtatgggaacaatgctgtgtggtgtgactccccaagaaggacctgctctatactacattgattcagacggcacccgtttacccggtaacctgttttgtgttggatccggtcagacattcgcgtacggtgtgctggactctaactaccgctacgatttgagcgaggaggaagctcttgctctgggccgtcgtgcgatcttggccgctatgcaccgtgatgcatactgcggcggcttcat

SEQ ID NO: 8 - DNA, 2086 bp, artificial
  Hairpin sequence
atgaagccgccgcagtatgcatcacggtgcatagcggccaagatcgcacgacggcccagagcaagagcttcctcctcgctcaaatcgtagcggtagttagagtccagcacaccgtacgcgaatgtctgaccggatccaacacaaaacaggttaccgggtaaacgggtgccgtctgaatcaatgtagtatagagcaggtccttcttggggagtcacaccacacagcattgttcccatactaaggccgtagcccttgtaagcataagtcaagttggcaagaatcttggatgcggccgccacgacccagctttcttgtacaaagtggtgatatcactagtgcggccgcctgcaggtcgaccatatggtcgacctgcaggcggccgcactagtgatgctgttatgttcagtgtcaagctgacctgcaaacacgttaaatgctaagaagttagaatatatgagacacgttaactggtatatgaataagctgtaaataaccgagtataaactcattaactaatatcacctctagagtataatataatcaaattcgacaatttgactttcaagagtaggctaatgtaaaatctttatatatttctacaatgttcaaagaaacagttgcatctaaacccctatggccatcaaattcaatgaacgctaagctgatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatcagcttaatatgactctcaataaagtctcataccaacaagtgccaccttattcaaccatcaagaaaaaagccaaaatttatgctactctaaggaaaacttcactaaagaagacgatttagagtgttttaccaagaatttctgtcatcttactaaacaactaaagatcggtgtgatacaaaacctaatctcattaaagtttatgctaaaataagcataattttacccactaagcgtgaccagataaacataactcagcacaccagagcatatatattggtggctcaaatcatagaaacttacagtgaagacacagaaagccgtaagaagaggcaagagtatgaaaccttacctcatcatttccatgaggttgcttctgatcccgcgggatatcaccactttgtacaagaaagctgggtcgtggcggccgcatccaagattcttgccaacttgacttatgcttacaagggctacggccttagtatgggaacaatgctgtgtggtgtgactccccaagaaggacctgctctatactacattgattcagacggcacccgtttacccggtaacctgttttgtgttggatccggtcagacattcgcgtacggtgtgctggactctaactaccgctacgatttgagcgaggaggaagctcttgctctgggccgtcgtgcgatcttggccgctatgcaccgtgatgcatactgcggcggcttcat

SEQ ID NO: 9 - DNA, 27 bp, artificial
  Primer
ccaccaccctggccttcmgnttycarg

SEQ ID NO: 10 - DNA, 23 bp, artificial
  Primer
ggccaccgccggnaaytggathg

SEQ ID NO: 11 - DNA, 24 bp, artificial
  Primer
gcggcggatcaccgtngcngcngc

SEQ ID NO: 12 - DNA, 21 bp, artificial
  Primer
cgtggccgccgcnwsnaarat

SEQ ID NO: 13 - DNA, 32 bp, artificial
  Primer
ggccaacctggtgtaccagtayaarggnatgg

SEQ ID NO: 14 - DNA, 23 bp, artificial
  Primer
tgggcctgtccatgggnacnatg

SEQ ID NO: 15 - DNA, 22 bp, artificial
  Primer
cccggtgcgtggcngcnardat

SEQ ID NO: 16 - DNA, 28 bp, artificial
  Primer
ggttgatgaagccgccgswrtangcrtc

SEQ ID NO: 17 - DNA, 29 bp, artificial
  Primer
ccagccctcctccttcacrtgrtanarrt

SEQ ID NO: 18 - DNA, 32 bp, artificial
  Primer
aactcctgcttctccaccttgryytyccaraa

SEQ ID NO: 19 - DNA, 49 bp, artificial
  Primer
ggggacaagtttgtacaaaaaagcaggctatgaagccgccgcagtatgc

SEQ ID NO: 20 - DNA, 49 bp, artificial
  Primer
ggggaccactttgtacaagaaagctgggtcgtggcggccgcatccaaga

SEQ ID NO: 21 - DNA, 19 bp, artificial
  Primer
caagattccttgccaactt

SEQ ID NO: 22 - DNA, 24 bp, artificial
  Primer
tgatgtaaccaccagagtaagcat

SEQ ID NO: 23 - DNA, 623 bp, Rhizoconia solani ZG3
ggaccgggccaagcaaagcgccgagtgggtgaggcatcaagagcaacagaaacaagaaaaggaagaccaggacgaacaggaacggattgaattcgcccttcagatcgactggcacgactttgtggtcgtggaaacagttttgttcactgagaatgatgaacacgatgatttgccgccaccaacctcgcttaacgacctgcagtctgcatctctcgagcaaaaagccgccatgtctcttaacccactgcggattgaggaggctatgccgaccgagcttgatgattccatctattacaatgcttatcctgtccaccatgaaaccgttcttacaccacagccctcctcccagcagcatgctccctccccggcctcatatctgccatccgcgcctgaccataatgcgcagcgcttatctgcattgccccttcacggccaacaagctcgcactgaatcgcccgttccgagtggtctaggccaaccgccaatgcgcattcgctccgactatgtcccccgagctcaagcccgtcgccagcaaggcgccaccgccatctgccccaattgccaccaacagatccctgtcgcagagctagatgagcatatgagtattgagctgctggaccccc

SEQ ID NO: 24 - protein, 207 aa, Rhizoconia solani ZG3
DRAKQSAEWVRHQEQQKQEKEDQDEQERIEFALQIDWHDFVVVETVLFTENDEHDDLPPPTSLNDLQSASLEQKAAMSLNPLRIEEAMPTELDDSIYYNAYPVHHETVLTPQPSSQQHAPSPASYLPSAPDHNAQRLSALPLHGQQARTESPVPSGLGQPPMRIRSDYVPRAQARRQQGATAICPNCHQQIPVAELDEHMSIELLDP

SEQ ID NO: 25 - DNA, 400 bp, Rhizoconia solani ZG3
tgactggcatgattttgtggttgtggaaacagttttgttcactgagaatgatgaacacgatgatttgccgccaccaacctcgcttaacgacctgcagtctgcatctctcgagcaaaaagccgccatgtctcttaacccactgcggattgaggaggctatgccgaccgagcttgatgattccatctattacaatgcttatcctgtccaccatgaaaccgttcttacaccacagccctcctcccagcagcatgctccctccccggcctcatatctgccatccgcgcctgaccataatgcgcagcgcttatctgcattgccccttcacggccaacaagctcgcactgaatcgcccgttccgagtggtctaggccaaccgccaatgcgcattcgctccgactat

SEQ ID NO: 26 - DNA, 2286 bp, artificial
  hairpin sequence
atagtcggagcgaatgcgcattggcggttggcctagaccactcggaacgggcgattcagtgcgagcttgttggccgtgaaggggcaatgcagataagcgctgcgcattatggtcaggcgcggatggcagatatgaggccggggagggagcatgctgctgggaggagggctgtggtgtaagaacggtttcatggtggacaggataagcattgtaatagatggaatcatcaagctcggtcggcatagcctcctcaatccgcagtgggttaagagacatggcggctttttgctcgagagatgcagactgcaggtcgttaagcgaggttggtggcggcaaatcatcgtgttcatcattctcagtgaacaaaactgtttccacaaccacaaaatcatgccagtcaacccagctttcttgtacaaagtggtgatatcactagtgcggccgcctgcaggtcgaccatatggtcgacctgcaggcggccgcactagtgatgctgttatgttcagtgtcaagctgacctgcaaacacgttaaatgctaagaagttagaatatatgagacacgttaactggtatatgaataagctgtaaataaccgagtataaactcattaactaatatcacctctagagtataatataatcaaattcgacaatttgactttcaagagtaggctaatgtaaaatctttatatatttctacaatgttcaaagaaacagttgcatctaaacccctatggccatcaaattcaatgaacgctaagctgatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatcagcttaatatgactctcaataaagtctcataccaacaagtgccaccttattcaaccatcaagaaaaaagccaaaatttatgctactctaaggaaaacttcactaaagaagacgatttagagtgttttaccaagaatttctgtcatcttactaaacaactaaagatcggtgtgatacaaaacctaatctcattaaagtttatgctaaaataagcataattttacccactaagcgtgaccagataaacataactcagcacaccagagcatatatattggtggctcaaatcatagaaacttacagtgaagacacagaaagccgtaagaagaggcaagagtatgaaaccttacctcatcatttccatgaggttgcttctgatcccgcgggatatcaccactttgtacaagaaagctgggttgactggcatgattttgtggttgtggaaacagttttgttcactgagaatgatgaacacgatgatttgccgccaccaacctcgcttaacgacctgcagtctgcatctctcgagcaaaaagccgccatgtctcttaacccactgcggattgaggaggctatgccgaccgagcttgatgattccatctattacaatgcttatcctgtccaccatgaaaccgttcttacaccacagccctcctcccagcagcatgctccctccccggcctcatatctgccatccgcgcctgaccataatgcgcagcgcttatctgcattgccccttcacggccaacaagctcgcactgaatcgcccgttccgagtggtctaggccaaccgccaatgcgcattcgctccgactat

SEQ ID NO: 27 - DNA, 25 bp, artificial
  Primer
tcgagaagaccgccggntaygtngc

SEQ ID NO: 28 - DNA, 27 bp, artificial
  Primer
gcacaggaacaaccccaarttywsntt

SEQ ID NO: 29 - DNA, 27 bp, artificial
  Primer
ggtgcagctgaccgccytntwygtngc

SEQ ID NO: 30 - DNA, 27 bp, artificial
  Primer
catgacccagctgatgcarmgngarrc

SEQ ID NO: 31 - DNA, 26 bp, artificial
  Primer
cagcgggagaccaggaayyhncartt

SEQ ID NO: 32 - DNA, 30 bp, artificial
  Primer
tcttccagcacatggtggaycartayrcna

SEQ ID NO: 33 - DNA, 24 bp, artificial
  Primer
ggaccgggccaagcarmgngcnga

SEQ ID NO: 34 - DNA, 29 bp, artificial
  Primer
gcaggagcagcagaagcaraarmargarg

SEQ ID NO: 35 - DNA, 29 bp, artificial
  Primer
cagatcgactggcacgacttygtngtngt

SEQ ID NO: 36 - DNA, 30 bp, artificial
  Primer
ggtggagaccgtcatcttcamngargcnga

SEQ ID NO: 37 - DNA, 28 bp, artificial
  Primer
cctgccacttgacccactcngcnckytg

SEQ ID NO: 38 - DNA, 30 bp, artificial
  Primer
gaagatgacggtctccaccacnacraartc

SEQ ID NO: 39 - DNA, 26 bp, artificial
  Primer
gggcaggttggcctggkcrtcngcyt

SEQ ID NO: 40 - DNA, 26 bp, artificial
  Primer
tgctggtggcagttgggrcanarngc

SEQ ID NO: 41 - DNA, 27 bp, artificial
  Primer
gggggtccagcagctcdatnckcatrt

SEQ ID NO: 42 - DNA, 28 bp, artificial
  Primer
ggactcggccttggccykytgytcyttc

SEQ ID NO: 43 - DNA, 24 bp, artificial
  Primer
ggaggccagccgcttnarrttrtt

SEQ ID NO: 44 - DNA, 49 bp, artificial
  Primer
ggggacaagtttgtacaaaaaagcaggctatagtcggagcgaatgcgca

SEQ ID NO: 45 - DNA, 49 bp, artificial
  Primer
ggggaccactttgtacaagaaagctgggttgactggcatgattttgtgg

SEQ ID NO: 46 - DNA, 989 bp, Rhizoctonia solani ZG3
atcacctggtgcggctttgaacacccgtccgaagtgcagcaggaatgcattcctcaggctgtgcttggcatggacgtactctgccaggccaaatctggtcatggcaagaccgcggaattcgcccttggcgacgttgcagcaactcgaaccggtggatggcgaggtgtcggtcattgtgctttgccacacacgagaacttgcttatcagatccgcaacgaatatttgcgcttctcgcgctttatgcctgatgtgcgcaccgcggtcgtgtatggcggtactccagttgcaaaggacattgagctcctcaaagacaagacaaaatgcccccacatcatcgtagccacccccggacgactcaacgcattggcccgtgacaagcatctcgacccaaagaaggtcaagcactttgtgttggacgagtgcgataaaatgttggagcaacttgacatgcgccgagacgttcaagaaattttccgagtcactcctcatcacaaacaagtcatgatgttcagcgctaccctgtccaaggatatccgcgtcacttgcaaaaaattcatggccaacccgctcgaaatctttatcgacgacgagtcgaagctcacccttcacggtctccagcaacactttttgaatctggaagaggcggcaaaaaaccgcaagctcaacgacctcttggatagtctggagtttaatcaggttgtcatcttcgtcaagtctgtttctcgagcaaatgagttgaacaaactccttaacaattgcaatttcccgagtatctgcatccactctggattgaaccaggaggagcgcattaaccgctaccaatctttcaaaaactttgagaagcgtattctagtcgccacggacatctttgggcgtggtattgacgtggaacgtgttaacattgttattaattacgacgccccctctgaggctgatagctacctgcatcgtgaagggcgaattcggagcttcggcaccaagggcctgg

SEQ ID NO: 47 - protein, 329 aa, Rhizoctonia solani ZG3
SPGAALNTRPKCSRNAFLRLCLAWTYSARPNLVMARPRNSPLATLQQLEPVDGEVSVIVLCHTRELAYQIRNEYLRFSRFMPDVRTAVVYGGTPVAKDIELLKDKTKCPHIIVATPGRLNALARDKHLDPKKVKHFVLDECDKMLEQLDMRRDVQEIFRVTPHHKQVMMFSATLSKDIRVTCKKFMANPLEIFIDDESKLTLHGLQQHFLNLEEAAKNRKLNDLLDSLEFNQVVIFVKSVSRANELNKLLNNCNFPSICIHSGLNQEERINRYQSFKNFEKRILVATDIFGRGIDVERVNIVINYDAPSEADSYLHREGRIRSFGTKGL

SEQ ID NO: 48 - DNA, 827 bp, Rhizoctonia solani ZG5
ggcgacgttgcagcaactcgaacctgtgaatggcgacgtatcggtaattgtgctttgccacacacgagaattggcataccaaattcgcaacgaatacatccgcttttctcgatacatgcctgatgtgaggaccggggttgtattcggtggtactccagtcgcgaaagacgtcgagctcctcaaagacaaggaaaagtgcccccacattatcgtggccacccccggacgactcaacgcattggcccgtgacaagcacctcgaccccaagaaggtcaagcactttgtgttggacgaatgcgataaaatgttggagcaacttgacatgcgccgagacgttcaagaaattttccgggtcactcctcaccacaagcaagtcatgatgttcagcgcaaccctgtccaaggacatccgtgtcacatgcaaaaaattcatggctaacccccttgaaatcttcatcgatgacgagtcgaagctcactcttcatggtcttcagcaatactttttgaacttggaggaggttggaaagaaccgcaaactcaacgatctcttggatcagttggagttcaaccaggttgtcatctttgtcaagtccgttacccgcgcgaacgaattgaacaagctcctcaacacctgcaactttccgagtatctgcatccactctggtttgaaccaggaggaacgtatctcacgttatcaatctttcaaaagttttgaaaagcgcattctcgtggcaaccgacatctttggtcgtggtattgacgtggagcgtgtcaacattgtcgtcaactacgacgctccttccgaggctgatagctacctgcatcgtg

SEQ ID NO: 49 - protein, 275 aa, Rhizoctonia solani ZG5
ATLQQLEPVNGDVSVIVLCHTRELAYQIRNEYIRFSRYMPDVRTGVVFGGTPVAKDVELLKDKEKCPHIIVATPGRLNALARDKHLDPKKVKHFVLDECDKMLEQLDMRRDVQEIFRVTPHHKQVMMFSATLSKDIRVTCKKFMANPLEIFIDDESKLTLHGLQQYFLNLEEVGKNRKLNDLLDQLEFNQVVIFVKSVTRANELNKLLNTCNFPSICIHSGLNQEERISRYQSFKSFEKRILVATDIFGRGIDVERVNIVVNYDAPSEADSYLHR

SEQ ID NO: 50 - DNA, 827 bp, Rhizoctonia solani rice infecting strain
ggcgacgttgcagcaactcgagcctgtcgacggagaggtctcggtcattgtgctatgccacacccgagagcttgcttaccaaatccgcaacgaatacacccgattctctcgatacatgcccgatgttcgaaccggcgtcgtgttcggtggtaccccagtcgcgaaggacatcgaactcctcaaagacaagaccaagtgtccccatatcattgtggctacccctggtcgcctcaacgcattggcccgtgacaagcatctcgaccccaagaaggtcaagcacttcgtgttggacgagtgcgataaaatgttggagcaacttgacatgcgtcgagacgttcaagaaattttcagagtcacgcctcatcacaagcaggtcatgatgttcagcgctaccctttccaaggatattcgtgtcacttgcaaaaagtttatggccaacccgcttgaaatattcattgacgacgagtcgaaactcaccctgcacggtcttcagcagcattacattaatttggaagaagtcgccaaaaaccgcaagcttaacgatcttttggatcaacttgagttcaaccaggttgtcatttttgtcaagtctgtctcccgggcgaacgaattgaacaagctcctcaacagctgcaacttcccaagtatctgcattcattctggtctcaaccaggaggagcgtattaaccgctatcagtccttcaaaagctttgaaaagcgtatccttgtggcgaccgatatcttcggccgtggtatcgacgttgaacgtgtgaacattgttgttaactatgatgcgccttccgaggctgatagctacctgcatcgtg

SEQ ID NO: 51 - protein, 275 aa, Rhizoctonia solani rice infecting strain
ATLQQLEPVDGEVSVIVLCHTRELAYQIRNEYTRFSRYMPDVRTGVVFGGTPVAKDIELLKDKTKCPHIIVATPGRLNALARDKHLDPKKVKHFVLDECDKMLEQLDMRRDVQEIFRVTPHHKQVMMFSATLSKDIRVTCKKFMANPLEIFIDDESKLTLHGLQQHYINLEEVAKNRKLNDLLDQLEFNQVVIFVKSVSRANELNKLLNSCNFPSICIHSGLNQEERINRYQSFKSFEKRILVATDIFGRGIDVERVNIVVNYDAPSEADSYLHR

SEQ ID NO: 52 - DNA, 400 bp, Rhizoctonia solani ZG3
cacatcatcgtagccacccccggacgactcaacgcattggcccgtgacaagcatctcgaccccaagaaggtcaagcactttgtgttggacgagtgcgataaaatgttggagcaacttgacatgcgccgagacgttcaagaaattttccgagtcactcctcatcacaagcaagtcatgatgttcagcgctaccctgtccaaggatatccgtgttacttgcaaaaaattcatggccaacccgctcgaaatcttcattgacgacgagtcgaagctcacacttcacggtcttcagcaacactttttgaatctggaagaggctgccaaaaaccgcaagctcaatgacctcttggatagtctggagtttaatcaggttgtcatcttcgtcaagtccgtttctcgag

SEQ ID NO: 53 - DNA, 2286 bp, Artificial
  hairpin sequence
ctcgagaaacggacttgacgaagatgacaacctgattaaactccagactatccaagaggtcattgagcttgcggtttttggcagcctcttccagattcaaaaagtgttgctgaagaccgtgaagtgtgagcttcgactcgtcgtcaatgaagatttcgagcgggttggccatgaattttttgcaagtaacacggatatccttggacagggtagcgctgaacatcatgacttgcttgtgatgaggagtgactcggaaaatttcttgaacgtctcggcgcatgtcaagttgctccaacattttatcgcactcgtccaacacaaagtgcttgaccttcttggggtcgagatgcttgtcacgggccaatgcgttgagtcgtccgggggtggctacgatgatgtgacccagctttcttgtacaaagtggtgatatcactagtgcggccgcctgcaggtcgaccatatggtcgacctgcaggcggccgcactagtgatgctgttatgttcagtgtcaagctgacctgcaaacacgttaaatgctaagaagttagaatatatgagacacgttaactggtatatgaataagctgtaaataaccgagtataaactcattaactaatatcacctctagagtataatataatcaaattcgacaatttgactttcaagagtaggctaatgtaaaatctttatatatttctacaatgttcaaagaaacagttgcatctaaacccctatggccatcaaattcaatgaacgctaagctgatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatcagcttaatatgactctcaataaagtctcataccaacaagtgccaccttattcaaccatcaagaaaaaagccaaaatttatgctactctaaggaaaacttcactaaagaagacgatttagagtgttttaccaagaatttctgtcatcttactaaacaactaaagatcggtgtgatacaaaacctaatctcattaaagtttatgctaaaataagcataattttacccactaagcgtgaccagataaacataactcagcacaccagagcatatatattggtggctcaaatcatagaaacttacagtgaagacacagaaagccgtaagaagaggcaagagtatgaaaccttacctcatcatttccatgaggttgcttctgatcccgcgggatatcaccactttgtacaagaaagctgggtcacatcatcgtagccacccccggacgactcaacgcattggcccgtgacaagcatctcgaccccaagaaggtcaagcactttgtgttggacgagtgcgataaaatgttggagcaacttgacatgcgccgagacgttcaagaaattttccgagtcactcctcatcacaagcaagtcatgatgttcagcgctaccctgtccaaggatatccgtgttacttgcaaaaaattcatggccaacccgctcgaaatcttcattgacgacgagtcgaagctcacacttcacggtcttcagcaacactttttgaatctggaagaggctgccaaaaaccgcaagctcaatgacctcttggatagtctggagtttaatcaggttgtcatcttcgtcaagtccgtttctcgag

SEQ ID NO: 54 - DNA, 30 bp, artificial
  Primer
ggacctgatcgactactccgaygargarht

SEQ ID NO: 55 - DNA, 30 bp, artificial
  Primer
cgacaagaagggctcctacgtnggnathca

SEQ ID NO: 56 - DNA, 26 bp, artificial
  Primer
atcacctggtgcggcttygarcaycc

SEQ ID NO: 57 - DNA, 25 bp, artificial
  Primer
ccaagtccggcctgggnaaracngc

SEQ ID NO: 58 - DNA, 28 bp, artificial
  Primer
ccgtgttcgtgctgaccacnytncanca

SEQ ID NO: 59 - DNA, 24 bp, artificial
  Primer
cacccgggagctggcntwycarat

SEQ ID NO: 60 - DNA, 26 bp, artificial
  Primer
gacgtcaagaccggcgtnttytwygg

SEQ ID NO: 61 - DNA, 26 bp, artificial
  Primer
ccccacatcatcgtgggnacnccngg

SEQ ID NO: 62 - DNA, 26 bp, artificial
  Primer
gacatgcggagggacgtncarganrt

SEQ ID NO: 63 - DNA, 29 bp, artificial
  Primer
cacccctcagcagaagcargtnatgatgt

SEQ ID NO: 64 - DNA, 21 bp, artificial
  Primer
cagccggccgggngtnccnac

SEQ ID NO: 65 - DNA, 31 bp, artificial
  Primer
ggaagatctcctgcacgtccsknckcatrtc

SEQ ID NO: 66 - DNA, 25 bp, artificial
  Primer
gggtggcggagaacatcatnacytg

SEQ ID NO: 67 - DNA, 33 bp, artificial
  Primer
ccttctcctccagcttgatgtartaytgytgna

SEQ ID NO: 68 - DNA, 29 bp, artificial
  Primer
ttgaactgcaggtcgtccarnarytcrtt

SEQ ID NO: 69 - DNA, 25 bp, artificial
  Primer
cggcgatggaggggaarttrcaytc

SEQ ID NO: 70 - DNA, 31 bp, artificial
  Primer
actccttgtaccgcttgatcckytcytcytg

SEQ ID NO: 71 - DNA, 25 bp, artificial
  Primer
cgaacacgtcggtggcnacrcadat

SEQ ID NO: 72 - DNA, 25 bp, artificial
  Primer
cggcgggcatgtcgtarttdatngc

SEQ ID NO: 73 - DNA, 25 bp, artificial
  Primer
ccaggcccttggtgccraanckncc

SEQ ID NO: 74 - DNA, 25 bp, artificial
  Primer
gcagggccacctcgaanckyttytc

SEQ ID NO: 75 - DNA, 49 bp, artificial
  Primer
ggggacaagtttgtacaaaaaagcaggctctcgagaaacggacttgacg

SEQ ID NO: 76 - DNA, 49 bp, artificial
  Primer
ggggaccactttgtacaagaaagctgggtcacatcatcgtagccacccc

SEQ ID NO: 77 - DNA, 20 bp, artificial
  Primer
ggcgacgttgcagcaactcg

SEQ ID NO: 78 - DNA, 20 bp, artificial
  Primer
cacgatgcaggtagctatca

SEQ ID NO: 79 - DNA, 470 bp, Rhizoctonia solani ZG3
cgggcgcagctggaaatgctacagaagcgcgagaagcatttggaacatcagattgctgaacaagatgctgcagcacgcaagcatgtcaacacaaataaaaacgctgccaaggccgcccttcgacggaagcacgccctcgagaaaaatctcgagcagacatcgggacaaataatgcagcttgagcaacaagtatactctatcgaagcggcgaatatcaatcacgagacattgcaagcgatgaaacaagccggtgcggccatggagcagatccacggtggaatgagcatcgaccaggtcgacgagactatggacgtactccgggaacaacatcaactcgccgacgatatcggcgccgcaataacatccgtgccatttggcgaccaggtggacgagggtgatcttgaagctgagttggaaggaatggaacaggaggctatggacgagcgcatttttaataccggcaccgtgcc

SEQ ID NO: 80 - protein, 156 aa, Rhizoctonia solani ZG3
RAQLEMLQKREKHLEHQIAEQDAAARKHVNTNKNAAKAALRRKHALEKNLEQTSGQIMQLEQQVYSIEAANINHETLQAMKQAGAAMEQIHGGMSIDQVDETMDVLREQHQLADDIGAAITSVPFGDQVDEGDLEAELEGMEQEAMDERIFNTGTV

SEQ ID NO: 81 - DNA, 400 bp, Rhizoctonia solani ZG3
acatcagattgctgaacaagatgctgcagcacgcaagcatgtcaacacaaataaaaacgctgccaaggccgcccttcgacggaagcacgccctcgagaaaaatctcgagcagacatcgggacaaataatgcagcttgagcaacaagtatactctatcgaagcggcgaatatcaatcacgagacattgcaagcgatgaaacaagccggtgcggccatggagcagatccacggtggaatgagcatcgaccaggtcgacgagactatggacgtactccgggaacaacatcaactcgccgacgatatcggcgccgcaataacatccgtgccatttggcgaccaggtggacgagggtgatcttgaagctgagttggaaggaatggaacaggaggctatggacgag

SEQ ID NO: 82 - DNA, 2286 bp, artificial
  hairpin sequence
ctcgtccatagcctcctgttccattccttccaactcagcttcaagatcaccctcgtccacctggtcgccaaatggcacggatgttattgcggcgccgatatcgtcggcgagttgatgttgttcccggagtacgtccatagtctcgtcgacctggtcgatgctcattccaccgtggatctgctccatggccgcaccggcttgtttcatcgcttgcaatgtctcgtgattgatattcgccgcttcgatagagtatacttgttgctcaagctgcattatttgtcccgatgtctgctcgagatttttctcgagggcgtgcttccgtcgaagggcggccttggcagcgtttttatttgtgttgacatgcttgcgtgctgcagcatcttgttcagcaatctgatgtacccagctttcttgtacaaagtggtgatatcactagtgcggccgcctgcaggtcgaccatatggtcgacctgcaggcggccgcactagtgatgctgttatgttcagtgtcaagctgacctgcaaacacgttaaatgctaagaagttagaatatatgagacacgttaactggtatatgaataagctgtaaataaccgagtataaactcattaactaatatcacctctagagtataatataatcaaattcgacaatttgactttcaagagtaggctaatgtaaaatctttatatatttctacaatgttcaaagaaacagttgcatctaaacccctatggccatcaaattcaatgaacgctaagctgatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatcagcttaatatgactctcaataaagtctcataccaacaagtgccaccttattcaaccatcaagaaaaaagccaaaatttatgctactctaaggaaaacttcactaaagaagacgatttagagtgttttaccaagaatttctgtcatcttactaaacaactaaagatcggtgtgatacaaaacctaatctcattaaagtttatgctaaaataagcataattttacccactaagcgtgaccagataaacataactcagcacaccagagcatatatattggtggctcaaatcatagaaacttacagtgaagacacagaaagccgtaagaagaggcaagagtatgaaaccttacctcatcatttccatgaggttgcttctgatcccgcgggatatcaccactttgtacaagaaagctgggtacatcagattgctgaacaagatgctgcagcacgcaagcatgtcaacacaaataaaaacgctgccaaggccgcccttcgacggaagcacgccctcgagaaaaatctcgagcagacatcgggacaaataatgcagcttgagcaacaagtatactctatcgaagcggcgaatatcaatcacgagacattgcaagcgatgaaacaagccggtgcggccatggagcagatccacggtggaatgagcatcgaccaggtcgacgagactatggacgtactccgggaacaacatcaactcgccgacgatatcggcgccgcaataacatccgtgccatttggcgaccaggtggacgagggtgatcttgaagctgagttggaaggaatggaacaggaggctatggacgag

SEQ ID NO: 83 - DNA, 28 bp, artificial
  Primer
ggaaggacacccccaagaaygcnathyt

SEQ ID NO: 84 - DNA, 23 bp, artificial
  Primer
cgggcgcagctgganatgytnca

SEQ ID NO: 85 - DNA, 31 bp, artificial
  Primer
cacctgcagaaccagatggangarcargang

SEQ ID NO: 86 - DNA, 29 bp, artificial
  Primer
cggaagaacgtgaccaccaayaarrmngc

SEQ ID NO: 87 - DNA, 26 bp, artificial
  Primer
accaacaagaacgccgcnaarrmngc

SEQ ID NO: 88 - DNA, 26 bp, artificial
  Primer
gcccagatcggcacgytngarcarca

SEQ ID NO: 89 - DNA, 23 bp, artificial
  Primer
gccatcgaggccgcnaayathaa

SEQ ID NO: 90 - DNA, 25 bp, artificial
  Primer
ggtgctgctcccgcarnkbrtccat

SEQ ID NO: 91 - DNA, 28 bp, artificial
  Primer
ccagctcgtcctccagctcnkyntcrtc

SEQ ID NO: 92 - DNA, 34 bp, artificial
  Primer
ttcagcatcttctcgtccatctsytcytgytsna

SEQ ID NO: 93 - DNA, 23 bp, artificial
  Primer
ggcacggtgccggtnttnarnat

SEQ ID NO: 94 - DNA, 25 bp, artificial
  Primer
gctcggcctcctcgtcntcytcytc

SEQ ID NO: 95 - DNA, 27 bp, artificial
  Primer
cagcttccgcagctcgkcytcytcrtc

SEQ ID NO: 96 - DNA, 49 bp, artificial
  Primer
ggggacaagtttgtacaaaaaagcaggctctcgtccatagcctcctgtt

SEQ ID NO: 97 - DNA, 49 bp, artificial
  Primer
ggggaccactttgtacaagaaagctgggtacatcagattgctgaacaag

SEQ ID NO: 98 - DNA, 902 bp, Rhizoctonia solani ZG3
cttccagctgcgggtgtataattataatacccacgaaaaggtcgctgcctttgaggctcatccggattatattcggtgtttaagcgtccacccggtcgcgagcttggttctgaccggaagcgatgatatgacgattaaagcctgggactgggacaaggggtggaagtgcgtccagctatacgaaggacacacccattacattatgaatattgctattaatcccaaggaccctaacacattcgccagcgcctgtctcgaccgcacagtcaaaatctggtctcttggcaatgcgactcccaatttcaccctcgatgcacacgaaaaaggcgtaaactatattgaatactaccatggcgccgataaaccctatattgttacgactggcgacgaccgtaccgtcaaggtatgggactaccacgccaaaagctgcatacagacactcgaggggcacaccgccaacgtctcttttgctattttccatccgcttttgccagtgatcgtttctggtagcgaagacggcacggtcaaaatctggcacgcgaatacctacaggctcgaaaacaccctgtcgtattctctcgaacgtgcgtggtgtgtgggctacaagcgaaacagcaacgatgtggccgttggctttgacgagggtgtggtggtggttaaactcgggagggaggaaccgagctttagcatggaccaggctgggaaaattgtctttgcacggaataacgaggtgatgggtgccaaccttcaagctgcgcaggaggatcctacgcctgaaggccaaaagttgcctcttgcgccacgcgagttgggaagcactgaaatcttcccatccaccatcgctcactctccaaacggccggttcgtggcagtttgtggtgacggcgagtacatcatctaca

SEQ ID NO: 99 - protein, 300 aa, Rhizoctonia solani ZG3
FQLRVYNYNTHEKVAAFEAHPDYIRCLSVHPVASLVLTGSDDMTIKAWDWDKGWKCVQLYEGHTHYIMNIAINPKDPNTFASACLDRTVKIWSLGNATPNFTLDAHEKGVNYIEYYHGADKPYIVTTGDDRTVKVWDYHAKSCIQTLEGHTANVSFAIFHPLLPVIVSGSEDGTVKIWHANTYRLENTLSYSLERAWCVGYKRNSNDVAVGFDEGVVVVKLGREEPSFSMDQAGKIVFARNNEVMGANLQAAQEDPTPEGQKLPLAPRELGSTEIFPSTIAHSPNGRFVAVCGDGEYIIY

SEQ ID NO: 100 - DNA, 651 bp, Rhizoctonia solani ZG5
gcctggaccggaccgtcaaaatatggtcacttggtaatgcgacccccaatttcaccctcgatgcacacgaaaaaggcgtaaactacgttgaatactatcatggcgccgataaaccgtacattgtcacgactggcgacgaccgtactgtcaaagtatgggactaccacgctaaaagctgcatacaaacactcgagggacacacggccaacgtctcttttgcgattttccacccgcttttaccagtcatcgtgtctggtagcgaggacgggacggtcaaaatttggcacgcgaatacctacaggctcgaaaacacgctatcatattcgctcgaacgcgcttggtgtgttggctacaagaaaagcagcaacgatgtggctgttggttttgatgagggcgtcgtggttgttaaacttggacgggaggagccgagctttagcatggaccaggctgggaagattgtgtttgcaaggaataatgaggtattgggtgcaaaccttcagactacgcaggaggatcctacacctgacggccaaaagctccctgttgccccacgcgaactaggaagcaccgaaatcttcccatccacgatagcacactctccaaacggcagatttgtggcagtttgtggtgacggcgagtacatcatctaca

SEQ ID NO: 101 - protein, 216 aa, Rhizoctonia solani ZG5
LDRTVKIWSLGNATPNFTLDAHEKGVNYVEYYHGADKPYIVTTGDDRTVKVWDYHAKSCIQTLEGHTANVSFAIFHPLLPVIVSGSEDGTVKIWHANTYRLENTLSYSLERAWCVGYKKSSNDVAVGFDEGVVVVKLGREEPSFSMDQAGKIVFARNNEVLGANLQTTQEDPTPDGQKLPVAPRELGSTEIFPSTIAHSPNGRFVAVCGDGEYIIY

SEQ ID NO: 102 - DNA, 532 bp, Rhizoctonia solani rice infecting strain
cttccagctgcgggtgtataattataatacccatgaaaaggtcgctgctttcgaggctcacccagactatatccggtgcttgagtgtccaccctgttgcaagtctggttctcacagggagtgacgatatgactatcaaggcttgggattgggataaggggtggaagtgtgtccagctatatgaagggcatacacactacatcatgaatattgccgttaatcccaaagaccccaatacctttgctagcgcctgtctcgaccgtactgtcaaagtctggtctctcggaaatccgacacccaatttcaccctcgatgctcacgagaaaggagtaaactatgtcgagtactaccacggcgccgataaaccctatatcgtcacaactggagacgatcgtaccgtcaaggtctgggattaccacgccaaaagctgtatacagacgctcgaagggcatacagccaacgtgtcttttgcgattttccacccgcttttacctgtaatcgtgtctggaagtgaagacggcaccatcaggatc

SEQ ID NO: 103 - protein, 177 aa, Rhizoctonia solani rice infecting strain
FQLRVYNYNTHEKVAAFEAHPDYIRCLSVHPVASLVLTGSDDMTIKAWDWDKGWKCVQLYEGHTHYIMNIAVNPKDPNTFASACLDRTVKVWSLGNPTPNFTLDAHEKGVNYVEYYHGADKPYIVTTGDDRTVKVWDYHAKSCIQTLEGHTANVSFAIFHPLLPVIVSGSEDGTIRI

SEQ ID NO: 104 - DNA, 400 bp, Rhizoctonia solani ZG3
ccggattatattcggtgtttaagcgtccacccggtcgcgagcttggttctgaccggaagcgatgatatgacgattaaagcctgggactgggacaaggggtggaagtgcgtccagctatacgaaggacacacccattacattatgaatattgctattaatcccaaggaccctaacacattcgccagcgcctgtctcgaccgcacagtcaaaatctggtctcttggcaatgcgactcccaatttcaccctcgatgcacacgaaaaaggcgtaaactatattgaatactaccatggcgccgataaaccctatattgttacgactggcgacgaccgtaccgtcaaggtatgggactaccacgccaaaagctgcatacagacactcgaggggcacaccgccaacg

SEQ ID NO: 105 - DNA, 2286 bp, artificial
  hairpin sequence
cgttggcggtgtgcccctcgagtgtctgtatgcagcttttggcgtggtagtcccataccttgacggtacggtcgtcgccagtcgtaacaatatagggtttatcggcgccatggtagtattcaatatagtttacgcctttttcgtgtgcatcgagggtgaaattgggagtcgcattgccaagagaccagattttgactgtgcggtcgagacaggcgctggcgaatgtgttagggtccttgggattaatagcaatattcataatgtaatgggtgtgtccttcgtatagctggacgcacttccaccccttgtcccagtcccaggctttaatcgtcatatcatcgcttccggtcagaaccaagctcgcgaccgggtggacgcttaaacaccgaatataatccggacccagctttcttgtacaaagtggtgatatcactagtgcggccgcctgcaggtcgaccatatggtcgacctgcaggcggccgcactagtgatgctgttatgttcagtgtcaagctgacctgcaaacacgttaaatgctaagaagttagaatatatgagacacgttaactggtatatgaataagctgtaaataaccgagtataaactcattaactaatatcacctctagagtataatataatcaaattcgacaatttgactttcaagagtaggctaatgtaaaatctttatatatttctacaatgttcaaagaaacagttgcatctaaacccctatggccatcaaattcaatgaacgctaagctgatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatcagcttaatatgactctcaataaagtctcataccaacaagtgccaccttattcaaccatcaagaaaaaagccaaaatttatgctactctaaggaaaacttcactaaagaagacgatttagagtgttttaccaagaatttctgtcatcttactaaacaactaaagatcggtgtgatacaaaacctaatctcattaaagtttatgctaaaataagcataattttacccactaagcgtgaccagataaacataactcagcacaccagagcatatatattggtggctcaaatcatagaaacttacagtgaagacacagaaagccgtaagaagaggcaagagtatgaaaccttacctcatcatttccatgaggttgcttctgatcccgcgggatatcaccactttgtacaagaaagctgggtccggattatattcggtgtttaagcgtccacccggtcgcgagcttggttctgaccggaagcgatgatatgacgattaaagcctgggactgggacaaggggtggaagtgcgtccagctatacgaaggacacacccattacattatgaatattgctattaatcccaaggaccctaacacattcgccagcgcctgtctcgaccgcacagtcaaaatctggtctcttggcaatgcgactcccaatttcaccctcgatgcacacgaaaaaggcgtaaactatattgaatactaccatggcgccgataaaccctatattgttacgactggcgacgaccgtaccgtcaaggtatgggactaccacgccaaaagctgcatacagacactcgaggggcacaccgccaacg

SEQ ID NO: 106 - DNA, 27 bp, artificial
  Primer
ggttcatcgcccggaaraaytggathg

SEQ ID NO: 107 - DNA, 27 bp, artificial
  Primer
cttccagctgcgggtgtayaaytayaa

SEQ ID NO: 108 - DNA, 27 bp, artificial
  Primer
ctacatccgggccatcgyngtncaycc

SEQ ID NO: 109 - DNA, 31 bp, artificial
  Primer
ccgacgacatgaccatcaagytntggraytg

SEQ ID NO: 110 - DNA, 26 bp, artificial
  Primer
gcctggaccggaccgtnaarathtgg

SEQ ID NO: 111 - DNA, 26 bp, artificial
  Primer
gcctggaccggaccgtnaarathtgg

SEQ ID NO: 112 - DNA, 27 bp, artificial
  Primer
cctgggctcctcccacscnaayttyac

SEQ ID NO: 113 - DNA, 26 bp, artificial
  Primer
aagggcgtgaaccacgtngaytayta

SEQ ID NO: 114 - DNA, 27 bp, artificial
  Primer
cctgctgaccacctccgaygaymrnac

SEQ ID NO: 115 - DNA, 27 bp, artificial
  Primer
gaccaagtcctgcatcgcnacnytnga

SEQ ID NO: 116 - DNA, 26 bp, artificial
  Primer
ttcgcctgctaccacccngarytncc

SEQ ID NO: 117 - DNA, 25 bp, artificial
  Primer
tctccggctccgaggayggnacnrt

SEQ ID NO: 118 - DNA, 30 bp, artificial
  Primer
cctgaactacggcctggagmgngcntggtg

SEQ ID NO: 119 - DNA, 28 bp, artificial
  Primer
gcacctgcgaggtctacccncaracnyt

SEQ ID NO: 120 - DNA, 23 bp, artificial
  Primer
gtggccgtgtgcggngayggnga

SEQ ID NO: 121 - DNA, 25 bp, artificial
  Primer
tgctgctgggcgtgamnggncargg

SEQ ID NO: 122 - DNA, 27 bp, artificial
  Primer
gatcctgatggtgccgtcytcnswncc

SEQ ID NO: 123 - DNA, 31 bp, artificial
  Primer
actgctcgaacctgtaggtgttnshrtkcca

SEQ ID NO: 124 - DNA, 28 bp, artificial
  Primer
cgttgggggagtggatcarngtytgngg

SEQ ID NO: 125 - DNA, 31 bp, artificial
  Primer
tgtagatgatgtactcgccgtcnccrcanac

SEQ ID NO: 126 - DNA, 28 bp, artificial
  Primer
ccttggagtcccacacgaartcnarngc

SEQ ID NO: 127 - DNA, 22 bp, artificial
  Primer
tgccgatgccgccytgnccnkt

SEQ ID NO: 128 - DNA, 31 bp, artificial
  Primer
gaccagtacacctgcttgggntcnacytcda

SEQ ID NO: 129 - DNA, 25 bp, artificial
  Primer
cgcaggcgatggtcacnarytcncc

SEQ ID NO: 130 - DNA, 31 bp, artificial
  Primer
ggtcgaagtgggagatggtgtangtytgrtc

SEQ ID NO: 131 - DNA, 29 bp, artificial
  Primer
ccgaaggaggtcacgttcacrtcyttrtc

SEQ ID NO: 132 - DNA, 26 bp, artificial
  Primer
ccgcagcaccacggtytgrtaytcna

SEQ ID NO: 133 - DNA, 25 bp, artificial
  Primer
gcgtcgcccacggtyttccayttrt

SEQ ID NO: 134 - DNA, 49 bp, artificial
  Primer
ggggacaagtttgtacaaaaaagcaggctcgttggcggtgtgcccctcg

SEQ ID NO: 135 - DNA, 49 bp, artificial
  Primer
ggggaccactttgtacaagaaagctgggtccggattatattcggtgttt

SEQ ID NO: 136 - DNA, 802 bp, Rhizoctonia solani ZG3
tgcggaacccactgccaccacactacaaagacatctcgaataccaatttgccagctgagctcctacctaagtataccaccatcgaatacacgctttctcgccctgcatccgtgccgcctattttcttatttgttgtcgatacatgcttggatgaagaggatttgaaggccctccgtgatgcattggtgcttagcttgagcttgctgccagcgcatgcattggtggggttaatcacgtacggtaccatgactcaagtacatgagttggggtatgctgaatgctctaagtcctacgtcttccgaggcggaaaagaatacacccctaaacaaatacaggacatgcttaacctgtcttcggccaaccgtgcggcccctcgcccaggtcagcccgtaccaccacaatcattcggcgccgctcggttcttgttgcccatttcccaagtcgagttccagcttacatcgatcctcgaacaactcgcccgtgacccatggccggttgccaacgacaaacgaccgttgagatgtactggtgtggcgattagtgtcgctgtagggcttttggagtcgacatttgcaaatacgggcgcgcgcattatggtcttctccggcggcccagctacagaaggacccggaatggttgttagcaatgaacttcgtgagcctattcggtcccaccatgatatcgaaagggacagcgttaagcacttcaaacgcgcctccaagttttacgaaggtctcgcgaagcgttccgcccaaaatggtcatgcgatcgacctgtttgccggctgcttcgaccaggtggg

SEQ ID NO: 137 - protein, 266 aa, Rhizoctonia solani ZG3
RNPLPPHYKDISNTNLPAELLPKYTTIEYTLSRPASVPPIFLFVVDTCLDEEDLKALRDALVLSLSLLPAHALVGLITYGTMTQVHELGYAECSKSYVFRGGKEYTPKQIQDMLNLSSANRAAPRPGQPVPPQSFGAARFLLPISQVEFQLTSILEQLARDPWPVANDKRPLRCTGVAISVAVGLLESTFANTGARIMVFSGGPATEGPGMVVSNELREPIRSHHDIERDSVKHFKRASKFYEGLAKRSAQNGHAIDLFAGCFDQV

SEQ ID NO: 138 - DNA, 805 bp, Rhizoctonia solani ZG3
tgcggaacccactgcccccacactataaggatatcacggagaacacaatacccccccgagctccacccgcaaagcaccacaatcgagtaccaattggcacgcccggcccccagcaccccccaatcttcctctatgttgtcgatacctgccaggaggatgacagtctccaggcgctgaaggactctctcatcatgagcttgtctcttttgccgcccaatgcgctagtcggtctgatcacatacggaactatggctcaagtacacgagctcggctacaccgagtgcgccaagtcttatgtattccgaggcaacaaggactatactgccaagcaggtgcaggaaatgctgggtctctcccatggtatccgtcccggtatccctcaacaacaaccagctcgcccccctcttcctccggccgcacgattccttcttcccgttcagcaggccgagttccaaattaccagcattctcgaacagctgcagcacgacccttggcccgtcgcaaacgataagcgtcccctgcgctgcaccggtgttgccctgagtgttgcggttggcttgttggagacctctttccaaaatgccggtgcccgtatcatgaactttgtcagcggtgccgctaccgaaggccctggccatgttgacacccctgagttaaaggagcctattcgttcacaccacgacattgaccgcgataacattaagtactacaagaaggctgtcaagttctacgatgccctcgccaagcgcgccgccaacaacggacatattgtcgacatcttcgcaggctgcttcgaccaggtggg

SEQ ID NO: 139 - protein, 267 aa, Rhizoctonia solani ZG3
RNPLPPHYKDITENTIPPRAPPAKHHNRVPIGTPGPQHPPIFLYVVDTCQEDDSLQALKDSLIMSLSLLPPNALVGLITYGTMAQVHELGYTECAKSYVFRGNKDYTAKQVQEMLGLSHGIRPGIPQQQPARPPLPPAARFLLPVQQAEFQITSILEQLQHDPWPVANDKRPLRCTGVALSVAVGLLETSFQNAGARIMNFVSGAATEGPGHVDTPELKEPIRSHHDIDRDNIKYYKKAVKFYDALAKRAANNGHIVDIFAGCFDQV

SEQ ID NO: 140 - DNA, 684 bp, Rhizoctonia solani ZG5
tgcggaacccactgccaccacactacaaggatatctcgaataccaatctgccagctgagctcctacccaagtataccaccatcgagtacacgctttctcgccctgcccccgtgccgcctattttcttgtttgttgtcgatacgtgcttggatgaggaggatttgaaggccctccgtgatgccttggtgctcagcttgagcttgctgccagcgcatgcattggtcgggttgatcacgtacggtaccatgacccaagtgcacgagctaggttatgctgaatgctccaagtcgtatgtcttccgaggcggaaaagaatacacccccaagcaaatccaagacatgcttaacctctcgtctgccaaccgcgcggcccctcgcccaggccagcccgtaccgccgcaatcattcggcgccgctcggttcttgttacccatttctcaggtcgaattccagcttacgtcgatcctcgaacaacttgcccgtgacccgtggccggttgccaacgacaagcgaccattgagatgcactggtgtggcgattagcgtcgcggtggggcttttggagtcgacatttgcaaatacaggcgcacgcatcatggtcttctccggtggtccagctacagaagggcctggaatggtcgtgagcaacgaacttcgcgagcccatgaggtcccaccacgacacga

SEQ ID NO: 141 - protein, 227 aa, Rhizoctonia solani ZG5
RNPLPPHYKDISNTNLPAELLPKYTTIEYTLSRPAPVPPIFLFVVDTCLDEEDLKALRDALVLSLSLLPAHALVGLITYGTMTQVHELGYAECSKSYVFRGGKEYTPKQIQDMLNLSSANRAAPRPGQPVPPQSFGAARFLLPISQVEFQLTSILEQLARDPWPVANDKRPLRCTGVAISVAVGLLESTFANTGARIMVFSGGPATEGPGMVVSNELREPMRSHHDT

SEQ ID NO: 142 - DNA, 942 bp, Rhizoctonia solani ZG3
cctggaggtgctgaccacaaaagaactaaaagtctctgggcttattggccatgccgtttcggccaacaaaaaatctgcatgcgttggcgaaacagagattggaattgcccaaacgtctgcttggaagatttgcacacttacacctagaacctccacggccgtctattttgaagttgtcacacccgctggccagccgcttcaagcgggttcccgaggtttgatccaatttgtgacacacttccagcactcgtcgggtcaaatgcgtctccgtgtctcgactattgcacgtaactttgccgaagcagggtcgcctacgatcgcagcgagctttgatcaggaggccgcagcggtgctcatggcccggatcgcagtgttcaaggccgaaagtgacggaattcgccctgtgctgcggtgggtggaccgtatgctcattcggttgtgtcaaaaattcgccgattatcgaaaggaggatacgatgagcttcagactggcggataactttagcatttatccacaatttatgttccatcttcgtcgtagccaattcttgcaggtgttcaacaatagtcctgacgagacagcattctataggcacatcctcaacgaagaggacgtaaacaactcgctcatcatgatgcaacctactctaatgtcgtatacctttgacacaccacctcaacccgtattgttggacagtgtctcgatcaaacccgacgttatccttctcctggatacttttttccacatcctgatcttccacgaagggcgaattctgcagaatggcgcaaggcaggataccaagagcaggacggctatgaaaatttccgggaactattggaagctcctgtgggcgatgcccaggacctccttgttgaccgattccctatcccacgttacgtcgtttgcgaccaggggggaagtcaagcccggttcctgtactcc

SEQ ID NO: 143 - protein, 313 aa, Rhizoctonia solani ZG3
LEVLTTKELKVSGLIGHAVSANKKSACVGETEIGIAQTSAWKICTLTPRTSTAVYFEVVTPAGQPLQAGSRGLIQFVTHFQHSSGQMRLRVSTIARNFAEAGSPTIAASFDQEAAAVLMARIAVFKAESDGIRPVLRWVDRMLIRLCQKFADYRKEDTMSFRLADNFSIYPQFMFHLRRSQFLQVFNNSPDETAFYRHILNEEDVNNSLIMMQPTLMSYTFDTPPQPVLLDSVSIKPDVILLLDTFFHILIFHEGRILQNGARQDTKSRTAMKISGNYWKLLWAMPRTSLLTDSLSHVTSFATRGEVKPGSCT

SEQ ID NO: 144 - DNA, 761 bp, Rhizoctonia solani ZG5
cctggaggtgctgaccacaaaagaactcaaagtctccgggctgattggtcacgctgtttcagccaataaaaagtctgcatgtgttggcgagacggagattggaattgcccaaacctctgcttggaagatttgcacgctcacacctaggacctccacagccgtctactttgaagtcgtcacccccgctggtcagccgcttcaagcgggttcgcgcggtttgatccaatttgtaacacacttccagcactcgtctggtcaaatgcgtctccgcgtctcgaccatcgcacgtaactttgctgaagcaggctcgcccacgatcgcagcaagctttgaccaagaggctgcagcggtattgatggcccgtatcgcagtgttcaaggccgaaattgacgatagcccggatgtcctccggtggttggatcgcatgctcattcgattgtgtcagaaattcgcagattaccgcaaggaggatacaatgagcttccgactggcggataactttagcatttatccacagtttatgttccatcttcgtcgtagtcagttcttgcaggtattcaacaatagtcctgacgagacagcgttttataggcacatcctcaacgaagaagacgtgaacaattcgctcatcatgatgcaacctactctaatgtcgtatacgtttgacacaccaccgcaacctgtattgttggacagcgtctcgatcaaacccgatgttatccttctcctggatacttttttccacatcctgatcttccacg

SEQ ID NO: 145 - protein, 253 aa, Rhizoctonia solani ZG5
LEVLTTKELKVSGLIGHAVSANKKSACVGETEIGIAQTSAWKICTLTPRTSTAVYFEVVTPAGQPLQAGSRGLIQFVTHFQHSSGQMRLRVSTIARNFAEAGSPTIAASFDQEAAAVLMARIAVFKAEIDDSPDVLRWLDRMLIRLCQKFADYRKEDTMSFRLADNFSIYPQFMFHLRRSQFLQVFNNSPDETAFYRHILNEEDVNNSLIMMQPTLMSYTFDTPPQPVLLDSVSIKPDVILLLDTFFHILIFH

SEQ ID NO: 146 - DNA, 356 bp, Rhizoctonia solani rice infecting strain
gctgcggtgggtggatagcatgctcattaggttatgccagaagtttgcggactatcgcaaagaagacacgatgagcttcagactagctgataactttagcatttatccgcaattcatgttccaccttcgccgtagtcagttcttgcaggtgttcaacaatagtcctgacgaaacagcgttctataggcatatcttgaatgaggaggacgtgaacaattcgctcattatgatgcaacctactctgatgtcgtacacttttgacacaccacctcagcccgtattgttggacagcgtctcgatcaaacccgacgttatccttctcctggatacttttttccacatcctgatcttccacg

SEQ ID NO: 147 - protein, 118 aa, Rhizoctonia solani rice infecting strain
LRWVDSMLIRLCQKFADYRKEDTMSFRLADNFSIYPQFMFHLRRSQFLQVFNNSPDETAFYRHILNEEDVNNSLIMMQPTLMSYTFDTPPQPVLLDSVSIKPDVILLLDTFFHILIFH

SEQ ID NO: 148 - DNA, 401 bp, Rhizoctonia solani ZG3
ttgatcaggaggccgcagcggtgctcatggcccggatcgcagtgttcaaggccgaaattgacgatagcccggatgttctgcggtggttggatcgtatgctcattcggttgtgtcaaaaattcgccgattatcgaaaggaggatacgatgagcttcagactggcggataactttagcatttatccacaatttatgttccatcttcgccgtagccaattcttgcaggtgttcaacaatagtcctgacgagacggcattctataggcacatcctcaacgaagaggacgtaaacaactcgctcatcatgatgcaacctactctaatgtcgtatacctttgacacaccacctcaacccgtattgttggacagtgtctcgatcaaacccgacgttatccttctcctg

SEQ ID NO: 149 - DNA, 2288 bp, artificial
  hairpin sequence
caggagaaggataacgtcgggtttgatcgagacactgtccaacaatacgggttgaggtggtgtgtcaaaggtatacgacattagagtaggttgcatcatgatgagcgagttgtttacgtcctcttcgttgaggatgtgcctatagaatgccgtctcgtcaggactattgttgaacacctgcaagaattggctacggcgaagatggaacataaattgtggataaatgctaaagttatccgccagtctgaagctcatcgtatcctcctttcgataatcggcgaatttttgacacaaccgaatgagcatacgatccaaccaccgcagaacatccgggctatcgtcaatttcggccttgaacactgcgatccgggccatgagcaccgctgcggcctcctgatcaaacccagctttcttgtacaaagtggtgatatcactagtgcggccgcctgcaggtcgaccatatggtcgacctgcaggcggccgcactagtgatgctgttatgttcagtgtcaagctgacctgcaaacacgttaaatgctaagaagttagaatatatgagacacgttaactggtatatgaataagctgtaaataaccgagtataaactcattaactaatatcacctctagagtataatataatcaaattcgacaatttgactttcaagagtaggctaatgtaaaatctttatatatttctacaatgttcaaagaaacagttgcatctaaacccctatggccatcaaattcaatgaacgctaagctgatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatcagcttaatatgactctcaataaagtctcataccaacaagtgccaccttattcaaccatcaagaaaaaagccaaaatttatgctactctaaggaaaacttcactaaagaagacgatttagagtgttttaccaagaatttctgtcatcttactaaacaactaaagatcggtgtgatacaaaacctaatctcattaaagtttatgctaaaataagcataattttacccactaagcgtgaccagataaacataactcagcacaccagagcatatatattggtggctcaaatcatagaaacttacagtgaagacacagaaagccgtaagaagaggcaagagtatgaaaccttacctcatcatttccatgaggttgcttctgatcccgcgggatatcaccactttgtacaagaaagctgggtttgatcaggaggccgcagcggtgctcatggcccggatcgcagtgttcaaggccgaaattgacgatagcccggatgttctgcggtggttggatcgtatgctcattcggttgtgtcaaaaattcgccgattatcgaaaggaggatacgatgagcttcagactggcggataactttagcatttatccacaatttatgttccatcttcgccgtagccaattcttgcaggtgttcaacaatagtcctgacgagacggcattctataggcacatcctcaacgaagaggacgtaaacaactcgctcatcatgatgcaacctactctaatgtcgtatacctttgacacaccacctcaacccgtattgttggacagtgtctcgatcaaacccgacgttatccttctcctg

SEQ ID NO: 150 - DNA, 28 bp, artificial
  Primer
gcatccggttctcctggaayrynttycc

SEQ ID NO: 151 - DNA, 24 bp, artificial
  Primer
gcccatcggcgccbtntayacncc

SEQ ID NO: 152 - DNA, 23 bp, artificial
  Primer
cgggcccggttctggmkntgycc

SEQ ID NO: 153 - DNA, 25 bp, artificial
  Primer
tgcggaacccactgccnscncayta

SEQ ID NO: 154 - DNA, 22 bp, artificial
  Primer
ccgccccgaccccnccnathtt

SEQ ID NO: 155 - DNA, 29 bp, artificial
  Primer
tggtgggcctgatcacctwyggnacnatg

SEQ ID NO: 156 - DNA, 30 bp, artificial
  Primer
cgagtgcgccaagtcctacrtnttymgngg

SEQ ID NO: 157 - DNA, 29 bp, artificial
  Primer
tgccacatcgtcgacatcttygcnggntg

SEQ ID NO: 158 - DNA, 24 bp, artificial
  Primer
cgacaacctgctgatgggnttyaa

SEQ ID NO: 159 - DNA, 29 bp, artificial
  Primer
cctggaggtgctgaccacnaargarytna

SEQ ID NO: 160 - DNA, 26 bp, artificial
  Primer
gcccagtccttcgaccargargcngc

SEQ ID NO: 161 - DNA, 24 bp, artificial
  Primer
gctgcggtgggtggaymgnatgyt

SEQ ID NO: 162 - DNA, 28 bp, artificial
  Primer
gctccaggacgttggtcadytgraaytc

SEQ ID NO: 163 - DNA, 28 bp, artificial
  Primer
cgatgtcgtggtgggaccknatnggytc

SEQ ID NO: 164 - DNA, 26 bp, artificial
  Primer
cccacctggtcgaagcanccngcraa

SEQ ID NO: 165 - DNA, 31 bp, artificial
  Primer
gtgaagttgcacaggttcttcatytcnarna

SEQ ID NO: 166 - DNA, 29 bp, artificial
  Primer
tgcttgaacatggaggtggtraanshrtc

SEQ ID NO: 167 - DNA, 21 bp, artificial
  Primer
ggcggcggcctcytgrtcraa

SEQ ID NO: 168 - DNA, 24 bp, artificial
  Primer
ccgcagcacgtcggsnccrtcrtc

SEQ ID NO: 169 - DNA, 29 bp, artificial
  Primer
cagccggatcagcatcckrtcnacccanc

SEQ ID NO: 170 - DNA, 27 bp, artificial
  Primer
cgggtcgtccttccggtartcngcraa

SEQ ID NO: 171 - DNA, 27 bp, artificial
  Primer
ccgcaggtggaacatgaaytgnggrta

SEQ ID NO: 172 - DNA, 26 bp, artificial
  Primer
tgccggtagaaggcggtytcrtcngg

SEQ ID NO: 173 - DNA, 30 bp, artificial
  Primer
tgtaggtgtccagggtgggytgdatcatna

SEQ ID NO: 174 - DNA, 31 bp, artificial
  Primer
cgtggaagatcaggatgtggaaraangtrtc

SEQ ID NO: 175 - DNA, 26 bp, artificial
  Primer
cagacgatgaaccggggnarnggrwa

SEQ ID NO: 176 - DNA, 27 bp, artificial
  Primer
ggagtacaggaaccgggcytgnswncc

SEQ ID NO: 177 - DNA, 49 bp, artificial
  Primer
ggggacaagtttgtacaaaaaagcaggctcaggagaaggataacgtcgg

SEQ ID NO: 178 - DNA, 49 bp, artificial
  Primer
ggggaccactttgtacaagaaagctgggtttgatcaggaggccgcagcg

SEQ ID NO: 179 - DNA, 546 bp, Rhizoctonia solani ZG3
gcaggacaacgtgcggtatgtgtatcagccattggacgagctctacattgtcctgattaccaaccgtcaatccaacatccttcaagatatcgacagtctgcacctatttgcccaggtgaccaccagcatttgcaagagcctcgacgagcgagagatcttgcgcaatgcatttgagctgttgagcgcattcgacgagttggtgactctgggttaccgggagaacctgtctttgtctcagataaagaccttccttgagatggagagtcacgaggagagaatccaagagatcattgaacggaacaaagaattggaggctagcgaagagcgaaagcgcaaggcaaagcaactggagatgcagcgcaaggaagctgctcgcacaggacgcagtgctgccccacgaaccccttcctaccccgtctatacacccccctcccgcccatcagtgccggatacatatgattcttatgaagcggagaaaaagaagacattcgccaagcccctcccaacccgcggcaagggatgcaattgggcaagaagtccaagacc

SEQ ID NO: 180 - protein, 181 aa, Rhizoctonia solani ZG3
QDNVRYVYQPLDELYIVLITNRQSNILQDIDSLHLFAQVTTSICKSLDEREILRNAFELLSAFDELVTLGYRENLSLSQIKTFLEMESHEERIQEIIERNKELEASEERKRKAKQLEMQRKEAARTGRSAAPRTPSYPVYTPPSRPSVPDTYDSYEAEKKKTFAKPLPTRGKGCNWARSPR

SEQ ID NO: 181 - DNA, 546 bp, Rhizoctonia solani rice infecting strain
gcaggacaacgtgcggtatgtttatcagccattggacgagctctacattgtcctgattaccaaccgtcaatccaacatccttcaagatatcgacagtctgcacctatttgcccaggtgaccaccagcatttgcaagagcctcgacgagcgagagatcttgcgcaatgcatttgagctgttgagcgcattcgacgagttggtgactctgggttaccgggagaacctgtctttgtctcagataaagaccttccttgagatggagagtcacgaggagagaatccaagagatcattgaacggaacaaagaattggaggctagcgaagagcgaaagcgcaaggcaaagcaactggagatgcagcgcaaggaagctgctcgcacaggacgcagtgctgccccacgaaccccttcctaccccgtctatacacccccctcccgcccatcagtgccggatacatatgattcttatgaagcggagaaaaagaagacattcgccaagcccctcccaacccgcggcaagggatgcaactgggcaagaagtccaagacc

SEQ ID NO: 182 - protein, 181 aa, Rhizoctonia solani rice infecting strain; identical to SEQ ID NO: 180

SEQ ID NO: 183 - DNA, 400 bp, Rhizoctonia solani ZG3
ccaaccgtcaatccaacatccttcaagatatcgacagtctgcacctatttgcccaggtgaccaccagcatttgcaagagcctcgacgagcgagagatcttgcgcaatgcatttgagctgttgagcgcattcgacgagttggtgactctgggttaccgggagaacctgtctttgtctcagataaagaccttccttgagatggagagtcacgaggagagaatccaagagatcattgaacggaacaaagaattggaggctagcgaagagcgaaagcgcaaggcaaagcaactggagatgcagcgcaaggaagctgctcgcacaggacgcagtgctgccccacgaaccccttcctaccccgtctatacacccccctcccgcccatcagtgccggatacatatga

SEQ ID NO: 184 - DNA, 2286 bp, artificial
  hairpin sequence
tcatatgtatccggcactgatgggcgggaggggggtgtatagacggggtaggaaggggttcgtggggcagcactgcgtcctgtgcgagcagcttccttgcgctgcatctccagttgctttgccttgcgctttcgctcttcgctagcctccaattctttgttccgttcaatgatctcttggattctctcctcgtgactctccatctcaaggaaggtctttatctgagacaaagacaggttctcccggtaacccagagtcaccaactcgtcgaatgcgctcaacagctcaaatgcattgcgcaagatctctcgctcgtcgaggctcttgcaaatgctggtggtcacctgggcaaataggtgcagactgtcgatatcttgaaggatgttggattgacggttggacccagctttcttgtacaaagtggtgatatcactagtgcggccgcctgcaggtcgaccatatggtcgacctgcaggcggccgcactagtgatgctgttatgttcagtgtcaagctgacctgcaaacacgttaaatgctaagaagttagaatatatgagacacgttaactggtatatgaataagctgtaaataaccgagtataaactcattaactaatatcacctctagagtataatataatcaaattcgacaatttgactttcaagagtaggctaatgtaaaatctttatatatttctacaatgttcaaagaaacagttgcatctaaacccctatggccatcaaattcaatgaacgctaagctgatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatcagcttaatatgactctcaataaagtctcataccaacaagtgccaccttattcaaccatcaagaaaaaagccaaaatttatgctactctaaggaaaacttcactaaagaagacgatttagagtgttttaccaagaatttctgtcatcttactaaacaactaaagatcggtgtgatacaaaacctaatctcattaaagtttatgctaaaataagcataattttacccactaagcgtgaccagataaacataactcagcacaccagagcatatatattggtggctcaaatcatagaaacttacagtgaagacacagaaagccgtaagaagaggcaagagtatgaaaccttacctcatcatttccatgaggttgcttctgatcccgcgggatatcaccactttgtacaagaaagctgggtccaaccgtcaatccaacatccttcaagatatcgacagtctgcacctatttgcccaggtgaccaccagcatttgcaagagcctcgacgagcgagagatcttgcgcaatgcatttgagctgttgagcgcattcgacgagttggtgactctgggttaccgggagaacctgtctttgtctcagataaagaccttccttgagatggagagtcacgaggagagaatccaagagatcattgaacggaacaaagaattggaggctagcgaagagcgaaagcgcaaggcaaagcaactggagatgcagcgcaaggaagctgctcgcacaggacgcagtgctgccccacgaaccccttcctaccccgtctatacacccccctcccgcccatcagtgccggatacatatga

SEQ ID NO: 185 - DNA, 27 bp, artificial
  Primer
gcaggacaacgtgcggtwygtntayca

SEQ ID NO: 186 - DNA, 24 bp, artificial
  Primer
ccagcccctggacgarytntayat

SEQ ID NO: 187 - DNA, 28 bp, artificial
  Primer
tcaccaacaggcagtccaayathytnca

SEQ ID NO: 188 - DNA, 28 bp, artificial
  Primer
ggcagtccaacatcctgcargayathga

SEQ ID NO: 189 - DNA, 32 bp, artificial
  Primer
caacacctgcaagaacatcgaygarmgngara

SEQ ID NO: 190 - DNA, 26 bp, artificial
  Primer
gagctggtgaccctgggntaymrnga

SEQ ID NO: 191 - DNA, 32 bp, artificial
  Primer
aacctgaccatcacccagatcmaracnttyyt

SEQ ID NO: 192 - DNA, 28 bp, artificial
  Primer
gctccttgttccgctcgatdatytcytg

SEQ ID NO: 193 - DNA, 24 bp, artificial
  Primer
ccgctcctcggtggcytcnarytc

SEQ ID NO: 194 - DNA, 25 bp, artificial
  Primer
gcttggccttccgcttnckytcytc

SEQ ID NO: 195 - DNA, 30 bp, artificial
  Primer
ggtcttggacttcttgcccarytgcatncc

SEQ ID NO: 196 - DNA, 25 bp, artificial
  Primer
tggcgtgcaggtccaryttnadytt

SEQ ID NO: 197 - DNA, 25 bp, artificial
  Primer
ccacgttggggtgggtnckraaytg

SEQ ID NO: 198 - DNA, 26 bp, artificial
  Primer
gggcccttgttcacccanacngtraa

SEQ ID NO: 199 - DNA, 25 bp, artificial
  Primer
cgccggtcagctcgtaytcnacngt

SEQ ID NO: 200 - DNA, 25 bp, artificial
  Primer
cctcgccggtcagctcrtaytcnac

SEQ ID NO: 201 - DNA, 25 bp, artificial
  Primer
cgccggacacctcgtanryngcrtc

SEQ ID NO: 202 - DNA, 25 bp, artificial
  Primer
cctcgtccacggtgccdatnbhcca

SEQ ID NO: 203 - DNA, 29 bp, artificial
  Primer
tgcatggggaagaagtcgtyytcrtcnhc

SEQ ID NO: 204 - DNA, 31 bp, artificial
  Primer
tggtcttgtcgaactgcacckkcatnggraa

SEQ ID NO: 205 - DNA, 49 bp, artificial
  Primer
ggggacaagtttgtacaaaaaagcaggcttcatatgtatccggcactga

SEQ ID NO: 206 - DNA, 49 bp, artificial
  Primer
ggggaccactttgtacaagaaagctgggtccaaccgtcaatccaacatc

SEQ ID NO: 207 - DNA, 1863 bp, Rhizoctonia solani ZG3
ggacgaccgggaccactttggaaaaaagcgcttggatctggcagggccactcctggctagtctcttccgtatgctctttaggaagctaaccaaggacgtatatcgctatctacaaaagtgtgttgaatcgaatcgcgaattccagcttcgtttagccgtcaaagctaacaccataactaacgggctaaaatattctcttgctacgggcaactggggcgaccagaagaagtccatgtcagccaaggcaggcgtctcccaggtcctgaatcggtacactttcgcatctaccttgtcgcatctcaggcgttgcaacacgcctcttggccgagaaggcaaaattgccaaaccacgccaacttcacaacacccattggggcatggtctgccccgcggagactccagaagggcaggcgtgtggtctggtgaaaaacttgtcattgatgtcctgcatttctgtcgggtcattatctggcccggtgatcgagtttttggaggaatggggcctggagagtctggaggagaacgtggcgaccggtaactccgcctctgcgaccaaagtatttgtcaacggcgtgtgggtgggcaagggcgaattccagcataatttggtcaagacattgcatcgactacggcgtatggaccacatcaattccgaagttagcattgttcgggatattcgtgagaaggagctacgtctttacacagatgccggccgtgtctgccgacccttattcattgtcgacgaacaacagaagctaacgctcaagaagaaacacgtcgagtggctacaaaacggagtcgatgaggaaggccaggaattcaaatggaagcagctagtcaagcgtggtgtgatcgaattgctcgatgcggaagaagaggagactgttatgatcagtatgactcccgaagatctcgaaaactcgagacgtatcgagggaggcggatatgtgtccaacgttaacgatcctgattgggatccttctggtacagcgcgtgtcaagagcgtacctaacatccacacttggacgcattgcgagattcatcccagtatgatcctcggcatttgcgcaagcattattccattcccggaccataaccaatcgcctcgtaacacctaccaatctgccatgggtaaacaagcaatgggcatttatctcacgaattatcagatccgtatggacaccatggcgaatatcctatattatcctcaaaagcccctggccaccaccaggacgatggagttcttgcgcttccgtgagttgcctgctggtcagaatgccattgtggccattgcttgttactcgggctacaaccaagaagattccgttgtcatgaaccaaagcagtattgatcgtggtcttttccgcagtctgttctaccgtacatacacagatgcagagaagaaggttggtttcaataccgtggagcacttcgagaagccctcgaggcagacgactgttggtatgcggaagggtacatacgataagcttgacgatgatggtatcatcgcccctggtgttcgtgtttcgggagaagacattatcatcggtaaaactgcaccattgaaccctgattctcaggagcttggtcagcgtaccagccagcacaccaagatcgacacttcaacacccttgcgtagtacagaaaacggtatcgtggaccaagttattgtgacatctgccaatgatggcctcacttttaccaaggttcgcatgcgtactaccaaggtcccgcagattggtgacaagttcgcttctcgtcacggtcaaaagggtactattggtattacttaccgacaagaagacatgcccttcactcgtgagggtgtcgtgcccgatctgattattaatccccacgccatccc

SEQ ID NO: 208 - protein, 620 aa, Rhizoctonia solani ZG3
DDRDHFGKKRLDLAGPLLASLFRMLFRKLTKDVYRYLQKCVESNREFQLRLAVKANTITNGLKYSLATGNWGDQKKSMSAKAGVSQVLNRYTFASTLSHLRRCNTPLGREGKIAKPRQLHNTHWGMVCPAETPEGQACGLVKNLSLMSCISVGSLSGPVIEFLEEWGLESLEENVATGNSASATKVFVNGVWVGKGEFQHNLVKTLHRLRRMDHINSEVSIVRDIREKELRLYTDAGRVCRPLFIVDEQQKLTLKKKHVEWLQNGVDEEGQEFKWKQLVKRGVIELLDAEEEETVMISMTPEDLENSRRIEGGGYVSNVNDPDWDPSGTARVKSVPNIHTWTHCEIHPSMILGICASIIPFPDHNQSPRNTYQSAMGKQAMGIYLTNYQIRMDTMANILYYPQKPLATTRTMEFLRFRELPAGQNAIVAIACYSGYNQEDSVVMNQSSIDRGLFRSLFYRTYTDAEKKVGFNTVEHFEKPSRQTTVGMRKGTYDKLDDDGIIAPGVRVSGEDIIIGKTAPLNPDSQELGQRTSQHTKIDTSTPLRSTENGIVDQVIVTSANDGLTFTKVRMRTTKVPQIGDKFASRHGQKGTIGITYRQEDMPFTREGVVPDLIINPHAI

SEQ ID NO: 209 - DNA, 1784 bp, Rhizoctonia solani ZG3
ggacgaccgggaccactttggaaaaaagcgcttggatctggcagggccactcctggctagtctcttccgtatgctctttaggaagctaaccaaggacgtatatcgctatctacaaaagtgtgttgaatcgaatcgcgaattccagcttcgtttagccgtcaaagctaacaccataactaacgggctaaaatattctcttgctacgggcaactggggcgaccagaagaagtccatgtcagccaaggcaggcgtctcccaggtcctgaatcggtacactttcgcatctaccttgtcgcatctcaggcgttgcaacacgcctcttggccgagaaggcaaaattgccaaaccacgccaacttcacaacacccattggggcatggtctgccccgcggagactccagaagggcaggcgtgtggtctggtgaaaaacttgtcattgatgtcctgcatttctgtcgggtcattatctggcccggtgatcgagtttttggaggaatggggcctggagagtctggaggagaacgtggcgaccggtaactccgcctctgcgaccaaagtatttgtcaacggcgtgtgggtgggcaagggcgaattccagcataatttggtcaagacattgcatcgactacggcgtatggaccacatcaattccgaagttagcattgttcgggatattcgtgagaaggagctacgtctttacacagatgccggccgtgtctgccgacccttattcattgtcgacgaacaacagaagctaacgctcaagaagaaacacgtcgagtggctacaaaacggagtcgatgaggaaggccaggaattcaaatggaagcagctagtcaagcgtggtgtgatcgaattgctcgatgcggaagaagaggagactgttatgatcagtatgactcccgaagatctcgaaaactcgagacgtatcgagggaggcggatatgtgtccaacgttaacgatcctgattgggatccttctggtacagcgcgtgtcaagagcgtacctaacatccacacttggacgcattgcgagattcatcccagtatgatcctcggcatttgcgcaagcattattccattcccggaccataaccaatcgcctcgtaacacctaccaatctgccatgggtaaacaagcaatgggcatttatctcacgaattatcagatccgtatggacaccatggcgaatatcctatattatcctcaaaagcctctcgcgacgacacgatcgatggagtacctcaagttccgagagcttccagctggtcaaaacgccatcgttgctattttgtgctacagcggttacaaccaggaggattccgttatcatgaaccagagttcgatcgacagaggcttgtttcgaagcatgtattatcgcagctacatggacatcgagaagaaggccggcgtcctgaatctggaggaattcgaaaagcctacccgggataccacactacgcatgaaacacggtacatacgacaagctggaacctgatggtttcatcgcgcccgggactggtgtcacaggtgaagatatcatcatcggcaagacggcaccacttcaagcggacagcgaagagctcgggcagcgtactcgcagccacaccaaacgcgacgtgtctacccccttgaaaagcacagaaaacggtattgtcgaccaagtgctcatcaccacaaatcacgaaggccacaaattcgtcaaagtccgtgtacgttcgacacgcgtccctcagactggggacaagttcgcttcgcgacacggtcaaaagggcaccatcggca

SEQ ID NO: 210 - protein, 594 aa, Rhizoctonia solani ZG3
DDRDHFGKKRLDLAGPLLASLFRMLFRKLTKDVYRYLQKCVESNREFQLRLAVKANTITNGLKYSLATGNWGDQKKSMSAKAGVSQVLNRYTFASTLSHLRRCNTPLGREGKIAKPRQLHNTHWGMVCPAETPEGQACGLVKNLSLMSCISVGSLSGPVIEFLEEWGLESLEENVATGNSASATKVFVNGVWVGKGEFQHNLVKTLHRLRRMDHINSEVSIVRDIREKELRLYTDAGRVCRPLFIVDEQQKLTLKKKHVEWLQNGVDEEGQEFKWKQLVKRGVIELLDAEEEETVMISMTPEDLENSRRIEGGGYVSNVNDPDWDPSGTARVKSVPNIHTWTHCEIHPSMILGICASIIPFPDHNQSPRNTYQSAMGKQAMGIYLTNYQIRMDTMANILYYPQKPLATTRSMEYLKFRELPAGQNAIVAILCYSGYNQEDSVIMNQSSIDRGLFRSMYYRSYMDIEKKAGVLNLEEFEKPTRDTTLRMKHGTYDKLEPDGFIAPGTGVTGEDIIIGKTAPLQADSEELGQRTRSHTKRDVSTPLKSTENGIVDQVLITTNHEGHKFVKVRVRSTRVPQTGDKFASRHGQKGTIG

SEQ ID NO: 211 - DNA, 650 bp, Rhizoctonia solani ZG3
cccctggccaccaccaggacgatggagttcttgcgcttccgtgagttgcctgctggtcagaatgccattgtggccattgcttgttactcgggctacaaccaagaagattccgttgtcatgaaccaaagcagtattgatcgtggtcttttccgcagtctgttctaccgtacatacacagatgcagagaagaaggttggtttcaataccgtggagcacttcgagaagccctcgaggcagacgactgttggtatgcggaagggtacatacgataagcttgacgatgatggtatcatcgcccctggtgttcgtgtttcgggagaagacattatcatcggtaaaactgcaccattgaaccctgattctcaggagcttggtcagcgtaccagccagcacaccaagatcgacacttcaacacccttgcgtagtacagaaaacggtatcgtggaccaagttattgtgacatctgccaatgatggcctcacttttaccaaggttcgcatgcgtactaccaaggtcccgcagattggtgacaagttcgcttctcgtcacggtcaaaagggtactattggtattacttaccgacaagaagacatgcccttcactcgtgagggtgtcgtgcccgatctgattattaatccccacgccatccc

SEQ ID NO: 212 - protein, 216 aa, Rhizoctonia solani ZG3
PLATTRTMEFLRFRELPAGQNAIVAIACYSGYNQEDSVVMNQSSIDRGLFRSLFYRTYTDAEKKVGFNTVEHFEKPSRQTTVGMRKGTYDKLDDDGIIAPGVRVSGEDIIIGKTAPLNPDSQELGQRTSQHTKIDTSTPLRSTENGIVDQVIVTSANDGLTFTKVRMRTTKVPQIGDKFASRHGQKGTIGITYRQEDMPFTREGVVPDLIINPHAI

SEQ ID NO: 213 - DNA, 767 bp, Rhizoctonia solani ZG5
catgtggacccactgcgagatacatcccagtatgatacttggtatctgcgcaagcatcattccattcccagatcataatcagtcacctcgtaatacctaccaatccgcaatgggtaaacaagcaatgggaatttacctgacgaattatcagatccgtatggacacaatggcgaatatcctctattatccccaaaaaccccttgcgacgacacgatcgatggaatacctcaagttccgagaacttcctgcgggtcaaaacgctatcgttgctattttgtgctacagcggttacaaccaggaagattccgtgattatgaaccagagctcgattgacaggggcttgttccgtagcatgtactatcgcagctacatggatatcgagaaaaaggccggcgtcttgaatctggaggaatttgaaaagcctactcgcgataccacactccgtatgaaacacggtacatatgacaaactggagcctgatggtttcatcgcgcctgggactggtgtaacgggcgaagatattattattggcaagacggcaccactccaggcggatagcgaggagcttgggcagcgtactcgtagccataccaaacgtgacgtttctactccgttgaaaagcacagagaacggcattgtcgaccaagtgctcatcacgacaaaccacgagggccacaaatttgtcaaagttcgcgtacgctcgacacgtgtccctcagactggggacaagttcgcttcgcggcacggtcaaaagggcaccatcggca

SEQ ID NO: 214 - protein, 255 aa, Rhizoctonia solani ZG5
MWTHCEIHPSMILGICASIIPFPDHNQSPRNTYQSAMGKQAMGIYLTNYQIRMDTMANILYYPQKPLATTRSMEYLKFRELPAGQNAIVAILCYSGYNQEDSVIMNQSSIDRGLFRSMYYRSYMDIEKKAGVLNLEEFEKPTRDTTLRMKHGTYDKLEPDGFIAPGTGVTGEDIIIGKTAPLQADSEELGQRTRSHTKRDVSTPLKSTENGIVDQVLITTNHEGHKFVKVRVRSTRVPQTGDKFASRHGQKGTIG

SEQ ID NO: 215 - DNA, 492 bp, Rhizoctonia solani rice infecting strain
cgtgctactccggctacaatcaggaggattcagtcattatgaatcaaagctcaatcgacagaggattgttccgtagcatgtactatcgaagctatatggatatcgagaagaaggctggcgtcttaaatttggaggagttcgaaaagcccacccgagataccacacttcgcatgaaacacggtacctatgacaagttggaagctgatggcttcatcgcacctggaactggtgtgacgggtgaagatattattatcggcaaaacagcccccttgcaggcagatagcgaagagcttggacagcgcactcgtagtcataccaaacgcgacgtttccactccgctcaagagcacagagaatggaattgttgaccaagtgcttattacgaccaaccacgagggtcacaagtttgtcaaagttcgtgtgcgttccacacgtgttccacagactggagacaagttcgcttcgcggcacggtcaaaagggcaccatcggca

SEQ ID NO: 216 - protein, 163 aa, Rhizoctonia solani rice infecting strain
CYSGYNQEDSVIMNQSSIDRGLFRSMYYRSYMDIEKKAGVLNLEEFEKPTRDTTLRMKHGTYDKLEADGFIAPGTGVTGEDIIIGKTAPLQADSEELGQRTRSHTKRDVSTPLKSTENGIVDQVLITTNHEGHKFVKVRVRSTRVPQTGDKFASRHGQKGTIG

SEQ ID NO: 217 - DNA, 400 bp, Rhizoctonia solani ZG3
ttagccgtcaaagctaacaccataactaacgggctaaaatattctcttgctacgggcaactggggcgaccagaagaagtccatgtcagccaaggcaggcgtctcccaggtcctgaatcggtacactttcgcatctaccttgtcgcatctcaggcgttgcaacacgcctcttggccgagaaggcaaaattgccaaaccacgccaacttcacaacacccattggggcatggtctgccccgcggagactccagaagggcaggcgtgtggtctggtgaaaaacttgtcattgatgtcctgcatttctgtcgggtcattatctggcccggtgatcgagtttttggaggaatggggcctggagagtctggaggagaacgtggcgaccggtaactccgcctctgcga

SEQ ID NO: 218 - DNA, 2286 bp, artificial
  hairpin sequence
tcgcagaggcggagttaccggtcgccacgttctcctccagactctccaggccccattcctccaaaaactcgatcaccgggccagataatgacccgacagaaatgcaggacatcaatgacaagtttttcaccagaccacacgcctgcccttctggagtctccgcggggcagaccatgccccaatgggtgttgtgaagttggcgtggtttggcaattttgccttctcggccaagaggcgtgttgcaacgcctgagatgcgacaaggtagatgcgaaagtgtaccgattcaggacctgggagacgcctgccttggctgacatggacttcttctggtcgccccagttgcccgtagcaagagaatattttagcccgttagttatggtgttagctttgacggctaaacccagctttcttgtacaaagtggtgatatcactagtgcggccgcctgcaggtcgaccatatggtcgacctgcaggcggccgcactagtgatgctgttatgttcagtgtcaagctgacctgcaaacacgttaaatgctaagaagttagaatatatgagacacgttaactggtatatgaataagctgtaaataaccgagtataaactcattaactaatatcacctctagagtataatataatcaaattcgacaatttgactttcaagagtaggctaatgtaaaatctttatatatttctacaatgttcaaagaaacagttgcatctaaacccctatggccatcaaattcaatgaacgctaagctgatccggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaaacgcgtggatcagcttaatatgactctcaataaagtctcataccaacaagtgccaccttattcaaccatcaagaaaaaagccaaaatttatgctactctaaggaaaacttcactaaagaagacgatttagagtgttttaccaagaatttctgtcatcttactaaacaactaaagatcggtgtgatacaaaacctaatctcattaaagtttatgctaaaataagcataattttacccactaagcgtgaccagataaacataactcagcacaccagagcatatatattggtggctcaaatcatagaaacttacagtgaagacacagaaagccgtaagaagaggcaagagtatgaaaccttacctcatcatttccatgaggttgcttctgatcccgcgggatatcaccactttgtacaagaaagctgggtttagccgtcaaagctaacaccataactaacgggctaaaatattctcttgctacgggcaactggggcgaccagaagaagtccatgtcagccaaggcaggcgtctcccaggtcctgaatcggtacactttcgcatctaccttgtcgcatctcaggcgttgcaacacgcctcttggccgagaaggcaaaattgccaaaccacgccaacttcacaacacccattggggcatggtctgccccgcggagactccagaagggcaggcgtgtggtctggtgaaaaacttgtcattgatgtcctgcatttctgtcgggtcattatctggcccggtgatcgagtttttggaggaatggggcctggagagtctggaggagaacgtggcgaccggtaactccgcctctgcga

SEQ ID NO: 219 - DNA, 27 bp, artificial
  Primer
cgagatccggtccgccntngaraargg

SEQ ID NO: 220 - DNA, 26 bp, artificial
  Primer
ggcgtggtgtccgacgrngayathyt

SEQ ID NO: 221 - DNA, 30 bp, artificial
  Primer
caggaacgactggcagatgytngaratgyt

SEQ ID NO: 222 - DNA, 26 bp, artificial
  Primer
gaccgggaggtggccytngayttyat

SEQ ID NO: 223 - DNA, 27 bp, artificial
  Primer
ggaggagggctgcgagacnmgnaargc

SEQ ID NO: 224 - DNA, 27 bp, artificial
  Primer
ggacgaccgggaccacttyggnaaraa

SEQ ID NO: 225 - DNA, 24 bp, artificial
  Primer
ccccctgctggccaanytnttymg

SEQ ID NO: 226 - DNA, 23 bp, artificial
  Primer
tccaaggccggcgtnwsncargt

SEQ ID NO: 227 - DNA, 29 bp, artificial
  Primer
ggagtacatggacgccgarsargargara

SEQ ID NO: 228 - DNA, 27 bp, artificial
  Primer
catgtggacccactgcgarathcaycc

SEQ ID NO: 229 - DNA, 27 bp, artificial
  Primer
cgcctccatcatccccttyccngayca

SEQ ID NO: 230 - DNA, 28 bp, artificial
  Primer
ccatggccaacatcctgtaytayccnca

SEQ ID NO: 231 - DNA, 26 bp, artificial
  Primer
cccctggccaccaccmgnwsnatgga

SEQ ID NO: 232 - DNA, 28 bp, artificial
  Primer
cgtgctactccggctacaaycargarga

SEQ ID NO: 233 - DNA, 27 bp, artificial
  Primer
gggcctgttccggtccytnttytwymg

SEQ ID NO: 234 - DNA, 22 bp, artificial
  Primer
cccggccgatgggngtrttngt

SEQ ID NO: 235 - DNA, 21 bp, artificial
  Primer
gcacaccaggccccartgngt

SEQ ID NO: 236 - DNA, 28 bp, artificial
  Primer
ccaggttcttcaccaggccrcangcytg

SEQ ID NO: 237 - DNA, 26 bp, artificial
  Primer
aagatgggctcggagggngtnccnac

SEQ ID NO: 238 - DNA, 24 bp, artificial
  Primer
gcccacccacacgccrttnayraa

SEQ ID NO: 239 - DNA, 24 bp, artificial
  Primer
gggccggcacacccknccngcrtc

SEQ ID NO: 240 - DNA, 31 bp, artificial
  Primer
ggtagaacagggaccggaacarnccnckrtc

SEQ ID NO: 241 - DNA, 25 bp, artificial
  Primer
gggcggtcttgccgatdatdatrtc

SEQ ID NO: 242 - DNA, 27 bp, artificial
  Primer
cttggtggtccgcatccknacyttnac

SEQ ID NO: 243 - DNA, 26 bp, artificial
  Primer
gaggcgaacttgtcgccdatytgngg

SEQ ID NO: 244 - DNA, 25 bp, artificial
  Primer
tgccgatggtgcccttytgnccrtg

SEQ ID NO: 245 - DNA, 23 bp, artificial
  Primer
gggatggcgtggggrttdatdat

SEQ ID NO: 246 - DNA, 28 bp, artificial
  Primer
ccttggacagctggcactcdatnarrtg

SEQ ID NO: 247 - DNA, 21 bp, artificial
  Primer
ggggccccgggcnckngcrtg

SEQ ID NO: 248 - DNA, 49 bp, artificial
  Primer
ggggacaagtttgtacaaaaaagcaggcttcgcagaggcggagttaccg

SEQ ID NO: 249 - DNA, 49 bp, artificial
  Primer
ggggaccactttgtacaagaaagctgggtttagccgtcaaagctaacac

SEQ ID NO: 250 - DNA, 26 bp, artificial
  Primer
ctytggttcatgayaacggaatcytc

SEQ ID NO: 251 - DNA, 23 bp, artificial
  Primer
atgatgtgcacgttctcggtcgt

SEQ ID NO: 252 - DNA, 17 bp, artificial
  Primer
tcagagttgagctgacc

(19) United States
(12) Patent Application Publication
(10) Pub. No.: US 2013/0160156 A1
(43) Pub. Date: Jun. 20, 2013

(54) METHOD FOR DOWN-REGULATING GENE EXPRESSION IN FUNGI

(71) Applicants: Simone Dorfmueller, Singapore (SG); Phuay-Yee Goh, Singapore (SG); Pascale Feldmann, Gent (BE)

(72) Inventors: Simone Dorfmueller, Singapore (SG); Phuay-Yee Goh, Singapore (SG); Pascale Feldmann, Gent (BE)

(73) Assignee: Devgen Private Limited, Singapore (SG)

(21) Appl. No.: 13/702,831

(22) PCT Filed: Jun. 8, 2011
(86) PCT No.: PCT/EP2011/059537

§ 371 Date: Feb. 28, 2013

(30) Foreign Application Priority Data
Jun. 8, 2010 (GB) 1009601.4

(51) Int. Cl. C12N 15/82; C12N 15/113; C07K 14/375; A01H 5/04; A01H 5/10; A01N 57/16; A01H 1/06

(52) U.S. Cl. CPC C12N 15/8282; A01N 57/16; C12N 15/113; A01H 1/06; A01H 5/04; A01H 5/10; C07K 14/375
USPC 800/265; 514/44 A; 536/24.5; 435/320.1; 435/252.3; 435/254.2; 435/418; 435/325; 435/417; 435/468; 800/301; 800/279; 536/23.74

(57) ABSTRACT

The present invention concerns methods for controlling and/or preventing fungus infestation on a cell, organism, substrate or material via dsRNA mediated gene silencing. The methods of the invention are particularly used to alleviate pathogenic fungal infestation on plants, plant materials or seeds. Suitable fungal target genes and fragments thereof, expression cassettes, dsRNA molecules, host cells expressing the dsRNA, compositions and transgenic plants and plant cells are provided.

### FIELD OF THE INVENTION

The invention relates to methods for preventing and controlling pathogenic fungus infestation of cells, organisms or substrates by silencing of an essential gene of a pathogenic fungus. The invention also relates to transgenic plants resistant to fungal infestation.

### BACKGROUND TO THE INVENTION

RNA interference or “RNAi” is a process of sequence-specific down-regulation (or inhibition) of gene expression (also referred to as “gene silencing” or “RNA-mediated gene silencing”) initiated by double-stranded RNA (dsRNA) that is complementary in sequence to a region of the target gene to be down-regulated (Fire, A. Trends Genet. Vol. 15, 358-363, 1999; Sharp, P. A. Genes Dev. Vol. 15, 485-490, 2001).

Over the last few years, down-regulation of target genes in multicellular organisms by means of RNA interference (RNAi) has become a well established technique. In general, RNAi comprises contacting the organism with a dsRNA fragment (generally either as two annealed complementary single-strands of RNA or as a hairpin construct) having a sequence that corresponds to at least part of a gene to be down-regulated (the “target gene”). Reference may be made to International application WO 99/32619 (Carnegie Institute of Washington), International application WO 00/01846 (Devgen), and to Fire et al., Nature, Vol. 391, pp.806-811, February 1998 for general description of the RNAi technique.

Cogoni and Macino, (1999) Nature. 399: 166-169 describe gene silencing by RNAi in the filamentous fungus Neurospora crassa. Gene silencing was achieved by transforming fungal cells with a transgene capable of expressing the dsRNA, allowing the dsRNA to be transcribed within cells of the fungus.

Liu et al., (2002) Genetics. 160: 463-470 describe RNA interference in the human pathogenic fungus Cryptococcus neoformans. Again, RNAi was achieved by transforming fungal cells in culture with a DNA construct encoding the dsRNA, such that the dsRNA was transcribed in situ in the fungal cells.

Kadotani et al., (2003) Mol Plant Microbe Interac. 16: 769-776 describe gene silencing in the ascomycete fungus Magnaporthe oryzae (formerly Magnaporthe grisea; anamorph Pyricularia oryzae Cav. and Pyricularia grisae), the causal agent of rice blast disease, by a mechanism having molecular features consistent with RNAi. Gene silencing was achieved by expression of dsRNA inside cells of the fungus: fungal protoplasts were transformed in the laboratory using DNA constructs capable of expressing the dsRNA, such that the dsRNA is transcribed inside of the fungal cell.

These studies confirm that RNA interference pathways are active in a number of different species of fungi.

Plant diseases are often a serious limitation on agricultural productivity and therefore have influenced the history and development of agricultural practices. A variety of pathogens are responsible for plant diseases, including pathogenic fungi and bacteria. Among the causal agents of infectious diseases of crop plants, however, fungi are the most economically important group of plant pathogens and are responsible for huge annual losses of marketable food, fiber, and feed.

Incidence of plant diseases has traditionally been controlled by agronomic practices that include crop rotation, the use of agrochemicals, and conventional breeding techniques. The use of chemicals to control plant pathogens, however, increases costs to farmers and causes harmful effects on the ecosystem. Consumers and government regulators alike are becoming increasingly concerned with the environmental hazards associated with the production and use of synthetic agrochemicals for protecting plants from pathogens. Because of such concerns, regulators have banned or limited the use of some of the most hazardous chemicals. The incidence of fungal diseases has been controlled to some extent by breeding resistant crops. Traditional breeding methods, however, are time-consuming and require continuous effort to maintain disease resistance as pathogens evolve. See, for example, Grover and Gowthaman (2003) Curr. Sci. 84:330-340. Thus, there is a significant need for novel alternatives for the control of plant pathogens that possess a lower risk of pollution and environmental hazards than is characteristic of traditional agrochemical-based methods and that are less cumbersome than conventional breeding techniques.

In light of the significant impact of plant pathogens, particularly fungal pathogens, on the yield and quality of crops, new methods for protecting plants from pathogens are needed.

Earlier, we have developed rice plants with enhanced resistance against fungal species, such as Magnaporthe oryzae, by genetically engineering rice to express antifungal dsRNA, whereby the dsRNA is taken up into the fungal cells and thereby down-regulates expression of the fungal target gene (International application WO 06/70227).

The present invention provides further solutions for controlling pathogenic fungi, particularly in rice and potato.

### BRIEF DESCRIPTION OF THE DRAWINGS

FIG. 1 Transgenic potato plantlets: A: grown in in vitro in agar; B: during acclimatization.

FIG. 2 Distribution of theRhizoctonia disease severity of transgenic potato events, compared to the vector and wild type (WT) controls.

FIG. 3 Results for transgenic target 1, 13 and 21 plants: disease severity (left axis, histograms) & plant vigour and incidence (right axis, black squares and grey lozenges), compared to wild type (WT).

### DESCRIPTION OF THE INVENTION

Methods for controlling fungal growth in or on a cell or an organism or for preventing fungal infestation of a cell or an organism susceptible to fungal infection are provided. Transformed plants, plant cells and seeds comprising a nucleotide sequence capable of targeting a fungal gene by RNA interference are further provided.

The methods of the invention can find practical application in any area of technology where it is desirable to cause a decreased growth, development, reproduction or survival of a pathogenic fungus. The methods of the invention further find practical application where it is desirable to specifically down-regulate expression of one or more target genes in a fungus. Particularly useful practical applications include, but are not limited to, (1) protecting plants against plant pathogenic fungi; (2) pharmaceutical or veterinary use in humans and animals (for example to control, treat or prevent fungal infections in humans); (3) protecting materials against damage caused by fungi; (4) protecting perishable materials (such as foodstuffs, seed, etc.) against damage caused by fungi.

The methods and transformed plants, plant cells and seeds are directed to induce pathogen resistance in plants, thereby protecting plants from pathogens, particularly pathogenic fungal resistance. A tissue-preferred promoter may be used to drive expression of an antipathogenic nucleotide sequence in specific plant tissues that are particularly vulnerable to fungal attack, such as, for example, the roots, leaves, stalks, vascular tissues, and seeds. Pathogen-inducible promoters may also be used to drive expression of the nucleic acids that cause RNA interference at or near the site of the fungal infection.

Methods of using compositions to protect cells, organisms or substrates from a pathogenic fungus are also provided, which comprise applying an antipathogenic composition to the environment of the pathogenic fungus by, for example, spraying, dusting, or coating.

The present disclosure provides a method for preventing and/or controlling pathogenic Rhizoctonia spp. infestation, comprising exposing a pathogenic Rhizoctonia to at least one dsRNA comprising annealed complementary strands, whereby ingestion of said dsRNA by said Rhizoctonia inhibits the expression of a Rhizoctonia target gene, which target gene comprises a nucleotide sequence that is complementary to at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120, 150, 200, 300, 400, 500, 600, 700, 800, 900 or 1000 contiguous nucleotides of one of the strands of said dsRNA. Inhibition of expression of a target gene can be by silencing of the target gene.

In accordance with one embodiment the invention relates to a method to obtain at least 20, 25, 30, 35, 40, 45, 50, 55, 60, 65, 70, 75, 80, 85, 90 or 95% Rhizoctonia mortality or at least 20, 25, 30, 35, 40, 45, 50, 55, 60, 65, 70, 75, 80, 85, 90 or 95% Rhizoctonia control by silencing of an essential gene of a pathogenic Rhizoctonia spp. by application of at least one dsRNA comprising annealed complementary strands, one of which comprises at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120, 150, 200, 300, 400, 500, 600, 700, 800, 900 or 1000 contiguous nucleotides that are complementary to a Rhizoctonia target gene essential to said Rhizoctonia spp., wherein said Rhizoctonia target gene
(i) is selected from the group of genes having a nucleotide sequence comprising any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(ii) is selected from the group of genes having a nucleotide sequence that, when the two sequences are optimally aligned and compared using the BLASTN alignment tool or using a global alignment based on approximate string matching as described herein, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(iii) is a fungal orthologue of a gene having a nucleotide sequence comprising any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, wherein the two orthologuous genes are similar in sequence to such a degree that when the two genes are optimally aligned and compared using the BLASTN alignment tool or using a global alignment based on approximate string matching as described herein, the orthologue has a sequence that is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of the sequences represented by SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or
(iv) is selected from the group of genes having a nucleotide sequence encoding an amino acid sequence that, when the two sequences are optimally aligned and compared using the BLASTP alignment tool or using a global alignment based on approximate string matching as described herein, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 2, 4, 6, 24, 47, 49, 51, 80, 99, 101, 103, 137, 139, 141, 143, 145, 147, 180, 182, 208, 210, 212, 214 or 216, or
(v) is selected from the group of genes having a nucleotide sequence encoding any of the amino acid sequences as represented in Table 6
   as compared to the control Rhizoctonia spp. applied with a dsRNA targeting a non-essential gene or a gene not naturally expressed in said pathogenic Rhizoctonia spp.

In the above method, the determination of the percentage fungus mortality or percentage fungus control is measured indirectly by scoring the percentage of contamination by the pathogenic fungus on an infected surface, wherein the surface can be any substrate, material, cell or organism including but not limited to the leaf surface of plants. The scoring of the percentage of contamination on an infected leaf surface of plants can vary between 0 to 100%; the intensity of the symptoms of contamination is determined according to biological criteria in function of the size and severity of lesions on the leaves (or leaf sheaths). 0% corresponds to an observation where no symptoms are visible on the leaf, so having a total absence of the pathogenic fungus. 100% corresponds to a leaf that is totally covered by a pathogenic fungus, meaning a brown and dried-out leaf (or death of the whole leaf). The result for a plant obtained by the average from a number of leaves from the same plant will normally vary between these extreme values. One may consider that above 30% contaminated leaf surface, the plant is very severely infested by the fungus (can be visualised as spots or lesions on the leaf surface, numerous in number). A percentage of less than 20% may be considered as a weak infestation (can be visualised as spots or lesions on the leaf surface, but very low in number) and between 10-15% as a very weak infestation (can be visualised as very light, small spots or lesions on the leaf surface, that are hardly visable). The percentages can be scored at different time intervals to determine the fungus mortality and/or fungus control after application of a nucleotide sequence that causes RNA interference targeting a fungal target gene essential to said pathogenic fungus compared with a nucleotide sequence targeting a non-essential gene or a gene not naturally expressed in said pathogenic fungus. As such, the above method can thus also be formulated as a method for controlling fungal infection or for decreasing the % fungal contamination (or infection) on leaf (or leaf sheath or stem) surface.

Methods for determining sequence identity are routine in the art and include use of the Blast software (BLASTN and BLASTP) and EMBOSS software (The European Molecular Biology Open Software Suite (2000), Rice, P. Longden, I. and Bleasby, A. Trends in Genetics 16, (6) pp 276-277). The term “identity” as used herein refers to the relationship between sequences at the nucleotide level. The expression “% identical” is determined by comparing optimally aligned sequences, e.g. two or more, over a comparison window wherein the portion of the sequence in the comparison window may comprise insertions or deletions as compared to the reference sequence for optimal alignment of the sequences. The reference sequence does not comprise insertions or deletions. The reference window is chosen from between at least 10 contiguous nucleotides to about 50, about 100 or to about 150 nucleotides, preferably between about 50 and 150 nucleotides. “% identity” is then calculated by determining the number of nucleotides that are identical between the sequences in the window, dividing the number of identical nucleotides by the number of nucleotides in the window and multiplying by 100. As a practical matter, whether any particular nucleic acid molecule is at least 95%, 96%, 97%, 98% or 99% identical to a reference nucleotide sequence refers to a comparison made between two molecules using standard algorithms well known in the art and can be determined conventionally using publicly available computer programs such as the BLASTN algorithm (see Altschul et al., Nucleic Acids Res. 25:3389-3402 (1997)). More recent software is provided by GenomeQuest™. GenomeQuest™ uses the GenePast Search based on percent identity wherein pairwise alignment between sequences is performed using a global alignment based on approximate string matching (Dufresne et al., Nature Biotechnology, 2002, Vol. 20, 1269-1271) with the goal of minimizing the number of errors (mismatches or gaps) in the alignment subject to the condition that the maximum number of errors allowed in each is YY % of the length of the query; wherein YY % =(100−XX) and wherein XX is the percentage identity specified. The length of the query is preferably the length whereby two sequences are optimally aligned. The length of the query can be seen as the comparison window, which is, for instance at least 100 bp, preferably at least 200 bp, 300 bp, 400 bp, 500 bp, 600 bp, 700 bp, 800 bp, 900 bp, 1000 bp, 1200 bp, 1400 bp, 1600 bp, 1800 bp, or at least 2000 bp. By way of an example of a 400 long query sequence on which a GenePAST search at 85% identity is run, the number of errors in an alignment (as a percentage of the length of the query), allowed in the alignment is (100−XX)=(100−85)=15%. This means that at maximum 15% of 400=60 errors are allowed in the alignment.

“Fungus control” refers to the limitation of damage caused by fungi on plants, cells, substrates or any other kind of material, in which the limitation can be established, e.g., by killing the fungus or by inhibiting the fungus' development, fertility or growth in such a manner that the fungus provides less damage, produces fewer offspring, is less fit or more susceptible to predator attack, or that the fungus is even deterred from feeding on such plants, cells, substrates or any other kind of material. In preferred embodiments described herein, the fungus is a Rhizoctonia spp.

“Silencing” (also referred to as “gene silencing” or “RNA-mediated gene silencing”) or “RNA interference” or “RNAi” is a process of sequence-specific down-regulation of gene expression (also called “gene suppression” or “inhibition of gene expression”) initiated by double-stranded RNA (dsRNA) that is complementary in sequence to a region of the target gene to be down-regulated. The term “silencing” refers to a measurable or observable reduction in gene expression or a complete abolition of detectable gene expression, at the level of protein product and/or mRNA product from the target gene, or at the level of phenotype. Down-regulation or inhibition of gene expression is “specific” when down-regulation or inhibition of the target gene occurs without manifest effects on other genes. Depending on the nature of the target gene, down-regulation or inhibition of gene expression in cells of a pest can be confirmed by phenotypic analysis of the cell or the whole pest or by measurement of mRNA or protein expression using molecular techniques such as RNA solution hybridization, nuclease protection, Northern hybridization, reverse transcription quantitative PCR, gene expression monitoring with a microarray, antibody binding, enzymelinked immunosorbent assay (ELISA), Western blotting, radioimmunoassay (RIA), other immunoassays, or fluorescence-activated cell analysis (FACS).

“Nucleotide sequence” as mentioned herein can be a DNA or an RNA sequence. For the purpose of obtaining an RNAi effect, the nucleotide sequence is preferably an RNA molecule that is capable of forming a double-stranded molecule, such as a dsRNA, siRNA or an RNA hairpin.

“Application” or “applying (with)” or “contacting (with)” or “exposing to” are used interchangeably throughout the application and can be, for example, spraying, dusting, or coating (such as the coating of seed). The fungal cell may be contacted with the nucleotide sequence (or nucleotide molecule having a nucleotide sequence) in any suitable manner, permitting direct uptake of the nucleotide sequence by the fungus. For example, the fungal cell can be contacted with the nucleotide sequence in pure or substantially pure form, for example an aqueous solution containing the nucleotide sequence. In this embodiment, the fungus may be simply “soaked” with an aqueous solution comprising the nucleotide sequence. In a further embodiment the fungal cell can be contacted with the nucleotide sequence by spraying the fungal cell with a liquid composition comprising the nucleotide sequence.

Alternatively, the nucleotide sequence may be linked to a food component of the fungus, such as a food component for a mammalian pathogenic fungus, or a plant cell or tissue for a plant pathogenic fungus in order to increase uptake of the nucleotide sequence by the fungus.

In other embodiments the fungal cell may be contacted with a composition containing the nucleotide sequence. The composition may, in addition to the nucleotide sequence, contain further excipients, diluents or carriers. Preferred features of such compositions are discussed in more detail below.

The nucleotide sequence may also be incorporated in the medium in which the fungus grows or in or on a material or substrate that is infested by the fungus, or may be impregnated in a substrate or material susceptible to infestation by the fungus.

A fungal “target gene” (alternatively called “target sequence” herein) or “an essential gene”, as used herein, is a gene the silencing of which causes a decreased growth, development, reproduction or survival of a pathogenic fungus. In one embodiment, the partial or complete silencing of an essential gene of a fungus results in significant fungus mortality or significant fungus control when such gene is silenced as compared to control fungi applied with a nucleotide sequence targeting a non-essential gene or a gene not naturally expressed in the fungus.

A gene ortholog (or an orthologous gene) is a similar gene in a different species likely evolved from a common ancestor and which normally has retained essentially the same function. Two sequences or genes (or parts thereof) which are “orthologous” or “similar”, as used herein, are similar in sequence to such a degree that when the two sequences are optimally aligned and compared using a global alignment based on approximate string matching as described herein, the sequences are at least 70%, 75%, preferably at least 85%, 90% or 95%, or most preferably between 96% and 100% identical to each other. Sequences or parts of sequences which have “high sequence identity”, as used herein, are sequences or parts of sequences wherein the number of positions with identical nucleotides, calculated when the sequences are optimally aligned and compared using a global alignment based on approximate string matching as described herein, is higher than 95%. A target gene, or at least a part therof, as used herein, preferably has high sequence identity to the nucleotide sequence (or a part thereof) of the invention in order for efficient gene silencing to take place in the target pest.

For the purpose of the invention, the “complement of a nucleotide sequence x” is the nucleotide sequence which would be capable of forming a double-stranded molecule with the represented nucleotide sequence, and which can be derived from the represented nucleotide sequence by replacing the nucleotides by their complementary nucleotide according to Chargaff's rules (A<>T; A<>U; G<>C) and reading the sequence in the 3′ to 5′ direction, i.e. in opposite direction, of the represented nucleotide sequence.

According to a further embodiment, the invention relates to a method for controlling pathogenic fungus infestation comprising contacting the pathogenic fungus with a ribonucleic acid that functions upon uptake by the fungus to inhibit the expression of a target sequence within said fungus, wherein said ribonucleic acid consists of a ribonucleotide sequence that is complementary to said target sequence, wherein said ribonucleotide sequence is transcribed from:
(i) a DNA sequence selected from the group of sequences comprising any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or sequences having, when the two sequences are optimally aligned and compared using the BLASTN alignment tool or using a global alignment based on approximate string matching as described herein, at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identity with any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(ii) a DNA sequence that is a fungal orthologue of a gene comprising any of the sequences as represented by SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, wherein the two sequences are similar in sequence to such a degree that when the two sequences are optimally aligned and compared using the BLASTN alignment tool or using a global alignment based on approximate string matching as described herein, said DNA sequence is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of the sequences represented by SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or
(iii) a DNA sequence selected from the group of sequences encoding an amino acid sequence that, when the two sequences are optimally aligned and compared using the BLASTP alignment tool or using a global alignment based on approximate string matching as described herein, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 2, 4, 6, 24, 47, 49, 51, 80, 99, 101, 103, 137, 139, 141, 143, 145, 147, 180, 182, 208, 210, 212, 214 or 216, or
(iv) a DNA sequence selected from the group of sequences encoding any of the amino acid sequences as represented in Table 6.

According to a specific embodiment, the invention relates to a method for preventing and/or controlling pathogenic Rhizoctonia spp. infestation, comprising exposing the pathogenic Rhizoctonia spp. to an agent comprising at least one dsRNA that functions upon uptake by the Rhizoctonia to inhibit the expression of a target gene within said Rhizoctonia, wherein said dsRNA comprises annealed complementary strands, one of which comprises a ribonucleotide sequence that is complementary to the sequence of said target gene, wherein said ribonucleotide sequence is transcribed from:
(i) a DNA sequence selected from the group of sequences comprising any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof, or a fragment thereof of at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120, 150, 200, 300, 400, 500, 600, 700, 800, 900 or 1000 contiguous nucleotides, or
(ii) a DNA sequence selected from the group of sequences having, when the two sequences are optimally aligned and compared using the BLASTN alignment tool, at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identity with any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof, or
(iii) a DNA sequence selected from the group of sequences encoding an amino acid sequence that, when the two sequences are optimally aligned and compared using the BLASTP alignment tool, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 47, 49, 51, 2, 4, 6, 24, 80, 99, 101, 103, 137, 139, 141, 143, 145, 147, 180, 182, 208, 210, 212, 214 or 216.

In the context of these methods it is understood that the length of said transcribed ribonucleotide sequence is at least 21 bp, preferably at least 22, 23, 24, 25, 30, 40, 50, 60, 70, 80, 90, 100 bp, more preferably at least 150, 200, 250, 300 by up to at least 500 bp. The transcribed ribonucleotide may form dsRNA portions, either with itself (e.g. hairpins) or with a second transcribed ribonucleotide sequence.

According to one embodiment, the methods of the invention rely on uptake into fungal cells of a ribonucleic acid or dsRNA or a nucleotide sequence as described herein present outside of the fungus (i.e. external to the cell wall) and do not require expression of a ribonucleic acid or dsRNA or a nucleotide sequence as described herein within the fungal cell. In addition, the present invention also encompasses methods as described herein wherein the fungal cell(s) is (are) contacted with an agent or a composition comprising any of the ribonucleic acids or the nucleotide sequences as described herein.

Said ribonucleotide sequence may be prepared in a manner known per se. For example, dsRNAs may be synthesised in vitro using chemical or enzymatic RNA synthesis techniques well known in the art. In one approach the two separate RNA strands may be synthesised separately and then annealed to form double-strands. Alternatively a hairpin is formed from one transcript. In a further embodiment, the dsRNA may be synthesised by intracellular expression in a host cell or organism from a suitable expression vector.

According to another embodiment, said ribonucleotide sequence or dsRNA or said nucleotide sequence may be expressed by a prokaryotic (for instance but not limited to a bacterial) or eukaryotic (for instance but not limited to a yeast) host cell or host organism, or a symbiotic organism (e.g. green algae or cyanbacterium).

According to a further embodiment, the present invention relates to a method for preventing and/or controlling the infection of a plant, part of a plant, reproductive plant material, seed or tuber(s) by a pathogenic Rhizoctonia spp., comprising expressing in or applying to said plant, part of a plant, reproductive plant material, seed or tuber(s) at least one dsRNA comprising annealed complementary strands, one of which comprises at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120, 150, 200, 300, 400, 500, 600, 700, 800, 900 or 1000 contiguous nucleotides that are complementary to a Rhizoctonia target gene essential to said Rhizoctonia, wherein said Rhizoctonia target gene
(i) is selected from the group of genes having a nucleotide sequence comprising any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(ii) is selected from the group of genes having a nucleotide sequence that, when the two sequences are optimally aligned and compared using the BLASTN alignment tool or using a global alignment based on approximate string matching as described herein, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(iii) is a fungal orthologue of a gene having a nucleotide sequence comprising any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, wherein the two orthologuous genes are similar in sequence to such a degree that when the two genes are optimally aligned and compared using the BLASTN alignment tool or using a global alignment based on approximate string matching as described herein, the orthologuous gene has a sequence that is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of the sequences represented by SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or
(iv) is selected from the group of genes having a nucleotide sequence encoding an amino acid sequence that, when the two sequences are optimally aligned and compared using the BLASTP alignment tool or using a global alignment based on approximate string matching as described herein, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 2, 4, 6, 24, 47, 49, 51, 80, 99, 101, 103, 137, 139, 141, 143, 145, 147, 180, 182, 208, 210, 212, 214 or 216, or
(v) is selected from the group of genes having a nucleotide sequence encoding any of the amino acid sequences as represented in Table 6.

The invention also applies to a method for preventing and/or controlling the infection of a substrate or material by a Rhizoctonia spp., comprising applying to said substrate or material at least one dsRNA comprising annealed complementary strands, one of which comprises at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120, 150, 200, 300, 400, 500, 600, 700, 800, 900 or 1000 contiguous nucleotides that are complementary to a Rhizoctonia target gene essential to said Rhizoctonia, wherein said Rhizoctonia target gene
(i) is selected from the group of genes having a nucleotide sequence comprising any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(ii) is selected from the group of genes having a nucleotide sequence that, when the two sequences are optimally aligned and compared using the BLASTN alignment tool or using a global alignment based on approximate string matching as described herein, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(iii) is a fungal orthologue of a gene having a nucleotide sequence comprising any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, wherein the two orthologuous genes are similar in sequence to such a degree that when the two genes are optimally aligned and compared using the BLASTN alignment tool or using a global alignment based on approximate string matching as described herein, the orthologuous gene has a sequence that is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of the sequences represented by SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or
(iv) is selected from the group of genes having a nucleotide sequence encoding an amino acid sequence that, when the two sequences are optimally aligned and compared using the BLASTP alignment tool tool or using a global alignment based on approximate string matching as described herein, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 2, 4, 6, 24, 47, 49, 51, 80, 99, 101, 103, 137, 139, 141, 143, 145, 147, 180, 182, 208, 210, 212, 214 or 216, or
(v) is selected from the group of genes having a nucleotide sequence encoding any of the amino acid sequences as represented in Table 6.

In one embodiment the fungus that causes unwanted damage to substrates or materials can be any of the fungi as described herein, but is preferably chosen from the group comprising the moulds, including but not limited to Stachybotrys spp., Aspergillus spp., Alternaria spp., Cladosporium spp., Penicillium spp. or Phanerochaete chrysosporium.

As used in the methods of the invention, fungal target genes, also called target sequences, comprise a sequence which is selected from the group of sequences comprising:
(i) a nucleotide sequence consisting of any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(ii) a nucleotide sequence that, when the two sequences are optimally aligned and compared using a global alignment based on approximate string matching as described herein, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(iii) a nucleotide sequence encoding an amino acid sequence that, when the two sequences are optimally aligned and compared based on approximate string matching as described herein, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 2, 4, 6, 24, 47, 49, 51, 80, 99, 101, 103, 137, 139, 141, 143, 145, 147, 180, 182, 208, 210, 212, 214 or 216, or
(iv) a nucleotide sequence encoding any of the amino acid sequences as represented in Table 6.

The invention thus relates to the silencing of one or more of the target genes listed herein, which can be target genes in Rhizoctonia species and also target genes in other fungal species.

The present invention further extends to any of the methods described herein wherein the fungal target gene is essential for the viability, growth, development or reproduction of the fungus; or wherein said fungal target gene is involved in the pathogenicity or infectivity of the fungus, preferably said fungal target gene is involved in the formation of germ tubes, conidia attachment, formation of appressoria, formation of the penetration peg or formation of conidia. Preferably, the fungal target gene is involved in any of the following non-limiting list of cellular functions: the fungal target gene is a gene involved in the function of a proteasome (subunit), spliceosome, helicase, intracellular transport, ESCRT pathway (endosomal sorting complex required for transport pathway), COPI vesicle coat (coat protein complex), GTPase activator activity (for example GTPase activator activity involved in ER to Golgi transport), δ-coatoamer or RNA polymerase II (subunit) (specified in Tables 2, 3 and 4).

According to another embodiment, the present invention relates to an (isolated) nucleic acid molecule selected from the group consisting of:
(i) a nucleic acid molecule which comprises at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120, 150, 200, 300, 400, 500, 600, 700, 800, 900 or 1000 contiguous nucleotides of a nucleotide sequence as represented by any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(ii) a nucleic acid molecule which comprises at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120, 150, 200, 300, 400, 500, 600, 700, 800, 900 or 1000 contiguous nucleotides of a nucleotide sequence that, when the two sequences are optimally aligned and compared using the BLASTN alignment tool or using a global alignment based on approximate string matching as described herein, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(iii) a nucleic acid molecule encoding an amino acid sequence that, when the two sequences are optimally aligned and compared using the BLASTP alignment tool or using a global alignment based on approximate string matching as described herein, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 2, 4, 6, 24, 47, 49, 51, 80, 99, 101, 103, 137, 139, 141, 143, 145, 147, 180, 182, 208, 210, 212, 214 or 216, or
(iv) a nucleic acid molecule encoding an amino acid sequence that, when the two sequences are optimally aligned and compared using the BLASTP alignment tool or using a global alignment based on approximate string matching as described herein, is at least 70%, 72%, 74%, 76%, 78%, 80%, 85%, 90% or 95% identical to any of SEQ ID NOs 24, 47, 80, 99, 101 or 143 over the total length of SEQ ID NOs 24, 47, 80, 99, 101 or 143, respectively, or
(v) a nucleic acid molecule encoding an amino acid sequence that, when the two sequences are optimally aligned and compared using the BLASTP alignment tool or using a global alignment based on approximate string matching as described herein, is at least 95%, 96%, 97%, 98% or 99% identical to SEQ ID NO 2, preferably over a length of at least 50, 60, 70, 80, 90, 100 or 105 amino acids, or
(vi) a nucleic acid molecule encoding an amino acid sequence that, when the two sequences are optimally aligned and compared using the BLASTP alignment tool or using a global alignment based on approximate string matching as described herein, is at least 91%, 92%, 93%, 94%, 95%, 96%, 97%, 98% or 99% identical to SEQ ID NO 4 or 6 over a length of at least 50, 60, 70, 80, 90 or 96 amino acids, or
(vii) a nucleic acid molecule encoding an amino acid sequence that, when the two sequences are optimally aligned and compared using the BLASTP alignment tool or using a global alignment based on approximate string matching as described herein, is at least 85%, 86%, 87%, 88%, 89%, 90%, 91%, 92%, 93%, 94%, 95%, 96%, 97%, 98% or 99% identical to SEQ ID NO 49 or 51, preferably over a length of at least 50, 75, 100, 125, 150, 175, 200, 225, 250 or 275 amino acids, or
(viii) a nucleic acid molecule encoding an amino acid sequence that, when the two sequences are optimally aligned and compared using the BLASTP alignment tool or using a global alignment based on approximate string matching as described herein, is at least 82%, 83%, 84%, 85%, 86%, 87%, 88%, 89%, 90%, 91%, 92%, 93%, 94%, 95%, 96%, 97%, 98% or 99% identical to SEQ ID NO 103 or 137, preferably over a length of at least 100, 120, 140, 160 or 177 amino acids, or
(ix) a nucleic acid molecule encoding an amino acid sequence that, when the two sequences are optimally aligned and compared using the BLASTP alignment tool or using a global alignment based on approximate string matching as described herein, is at least 86%, 87%, 88%, 89%, 90%, 91%, 92%, 93%, 94%, 95%, 96%, 97%, 98% or 99% identical to SEQ ID NO 139, preferably over a length of at least 50, 75, 100, 125, 150, 175, 200, 225, 250 or 270 amino acids, or
(x) a nucleic acid molecule encoding an amino acid sequence that, when the two sequences are optimally aligned and compared using the BLASTP alignment tool or using a global alignment based on approximate string matching as described herein, is at least 86%, 87%, 88%, 89%, 90%, 91%, 92%, 93%, 94%, 95%, 96%, 97%, 98% or 99% identical to SEQ ID NO 141, preferably over a length of 50, 75, 100, 125, 150, 175, 200 or 227 amino acids, or
(xi) a nucleic acid molecule encoding an amino acid sequence that, when the two sequences are optimally aligned and compared using the BLASTP alignment tool or using a global alignment based on approximate string matching as described herein, is at least 91%, 92%, 93%, 94%, 95%, 96%, 97%, 98% or 99% identical to SEQ ID NO 145, preferably over a length of at least 250, 300, 350, 400, 450, 500 or 530 amino acids, or
(xii) a nucleic acid molecule encoding an amino acid sequence that, when the two sequences are optimally aligned and compared using the BLASTP alignment tool or using a global alignment based on approximate string matching as described herein, is at least 94%, 95%, 96%, 97%, 98% or 99% identical to SEQ ID NO 147, preferably over a length of at least 50, 60, 70, 80, 90, 100 or 118 amino acids, or
(xiii) a nucleic acid molecule encoding an amino acid sequence that, when the two sequences are optimally aligned and compared using the BLASTP alignment tool or using a global alignment based on approximate string matching as described herein, is at least 92%, 93%, 94%, 95%, 96%, 97%, 98% or 99% identical to SEQ ID NO 180 or 182, preferably over a length of at least 50, 75, 100, 125, 150, 175 or 182 amino acids, or
(xiv) a nucleic acid molecule encoding an amino acid sequence that, when the two sequences are optimally aligned and compared using the BLASTP alignment tool or using a global alignment based on approximate string matching as described herein, is at least 80%, 81%, 82%, 83%, 84%, 85%, 86%, 87%, 88%, 89%, 90%, 91%, 92%, 93%, 94%, 95%, 96%, 97%, 98% or 99% identical to SEQ ID NO 208, preferably over a length of at least 250, 300, 350, 400, 450, 500, 550, 600 or 624 amino acids,
(xv) a nucleic acid molecule encoding an amino acid sequence that, when the two sequences are optimally aligned and compared using the BLASTP alignment tool or using a global alignment based on approximate string matching as described herein, is at least 85%, 86%, 87%, 88%, 89%, 90%, 91%, 92%, 93%, 94%, 95%, 96%, 97%, 98% or 99% identical to SEQ ID NO 210, preferably over a length of at least 250, 300, 350, 400, 450, 500, 550 or 596 amino acids, or
(xvi) a nucleic acid molecule encoding an amino acid sequence that, when the two sequences are optimally aligned and compared using the BLASTP alignment tool or using a global alignment based on approximate string matching as described herein, is at least 88%, 89%, 90%, 91%, 92%, 93%, 94%, 95%, 96%, 97%, 98% or 99% identical to SEQ ID NO 212, preferably over a length of 50, 75, 100, 125, 150, 175, 200 or 217 amino acids, or
(xvii) a nucleic acid molecule encoding an amino acid sequence that, when the two sequences are optimally aligned and compared using the BLASTP alignment tool or using a global alignment based on approximate string matching as described herein, is at least 93%, 94%, 95%, 96%, 97%, 98% or 99% identical to SEQ ID NO 214, preferably over a length of 50, 75, 100, 125, 150, 175, 200, 225 or 255 amino acids, or
(xviii) a nucleic acid molecule encoding an amino acid sequence that, when the two sequences are optimally aligned and compared using the BLASTP alignment tool or using a global alignment based on approximate string matching as described herein, is at least 91%, 92%, 93%, 94%, 95%, 96%, 97%, 98% or 99% identical to SEQ ID NO 216, preferably over a length of 50, 75, 100, 120, 140 or 163 amino acids.

The invention also relates to an isolated dsRNA comprising annealed complementary strands, one of which has a nucleotide sequence which is complementary to at least part of a target nucleotide sequence of a target gene of a fungus. The target gene may be any of the target genes described herein, or a part thereof that exerts the same function.

According to another embodiment the present invention relates to an isolated dsRNA comprising annealed complementary strands, one of which has a nucleotide sequence which comprises at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120, 150, 200, 300, 400, 500, 600, 700, 800, 900 or 1000 contiguous nucleotides that are complementary to a nucleotide sequence of a fungal target gene, wherein said target gene comprises a sequence which is selected from the group consisting of:
(i) the sequences consisting of any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(ii) sequences which, when the two sequences are optimally aligned and compared using a global alignment based on approximate string matching as described herein, are at least 70%, 75%, at least 80% or 85% identical, preferably at least 90%, 95%, 96%, or more preferably at least 97%, 98% and still more preferably at least 99% identical to a sequence represented by any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(iii) sequences comprising at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120 or 150 contiguous nucleotides of any of SEQ ID NOs 1, 3, 5, 7, 21, 22, 23, 25, 46, 48, 50, 52, 77, 78, 79, 81, 98, 100, 102, 104, 136, 138, 140, 142, 144, 146, 148, 179, 181, 183, 207, 209, 211, 213, 215, 217 or 250, or the complement thereof, or
(iv) sequences having a nucleotide sequence encoding an amino acid sequence that, when the two sequences are optimally aligned and compared based on approximate string matching as described herein, are at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 2, 4, 6, 24, 47, 49, 51, 80, 99, 101, 103, 137, 139, 141, 143, 145, 147, 180, 182, 208, 210, 212, 214 or 216, or
(v) sequences having a nucleotide sequence encoding any of the amino acid sequences as represented in Table 6.

The current invention also relates to an expression cassette comprising at least one regulatory sequence that directs the expression of at least one nucleic acid molecule selected from:
(i) a nucleic acid molecule as described above, or
(ii) a nucleic acid molecule having a first region containing a nucleotide sequence of a nucleic acid molecule as described herein identical to at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120 or 150 contiguous nucleotides of a selected target gene, wherein the target gene is pathogenic Rhizoctonia gene, and a second region which is complementary to the first region, said expression cassette being capable of expressing (or expressing) an RNA molecule, preferably a dsRNA, containing a nucleotide sequence identical to at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120 or 150 contiguous nucleotides of said pathogenic Rhizoctonia target gene, or
(iii) a nucleic acid molecule having a nucleotide sequence encoding an amino acid sequence that, when the two sequences are optimally aligned and compared using the BLASTP alignment tool or using a global alignment based on approximate string matching as described herein, is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to any of SEQ ID NOs 2, 4, 6, 24, 47, 49, 51, 80, 99, 101, 103, 137, 139, 141, 143, 145, 147, 180, 182, 208, 210, 212, 214 or 216, or
(iv) a nucleic acid molecule having a nucleotide sequence encoding any of the amino acid sequences as represented in Table 6.

The term “regulatory sequence” is explained later herein.

A plant expression cassette preferably contains regulatory sequences for expression in plants, which are operatively linked to the nucleotide sequence. Suitable plant expression vectors are known in the art and include pK7GWIWG2D(II) as further described in the examples section.

The invention also relates to a dsRNA molecule produced from the expression of a nucleic acid molecule described herein or produced from an expression cassette described herein.

As used in the methods of the invention, the nucleotide sequence or ribonucleotide sequence as described herein can be expressed from an expression cassette, which cassette comprises at least one regulatory sequence.

The present invention also relates to a host cell comprising at least one nucleic acid molecule described herein or expression cassette described herein or double-stranded RNA molecule described herein. The host cell can be any prokaryotic cell, including bacterial cells, or any eukaryotic cell, including but not limited to yeast cells, plant cells and animal cells. Also, in the methods of the invention, the nucleotide sequence or ribonucleotide sequence or dsRNA as described herein can be expressed by at least one prokaryotic or eukaryotic host cell or host organism. The prokaryotic cell can be a bacterial cell chosen from the group comprising, but not limited to, Gram positive and Gram negative cells comprising Escherichia spp. (e.g. E. coli), Bacillus spp. (e.g. B. thuringiensis), Rhizobium spp., Lactobacilllus spp., Lactococcus spp., Pseudomonas spp. and Agrobacterium spp. The bacterial cell can be inactivated by heat or by chemical treatment before application. The eukaryotic cell or organism can be for instance yeast, preferably chosen from Pichia spp. (e.g. P. pastoris) and Saccharomyces spp. (e.g. S. cerevisiae); or can be a plant cell or a plant.

If the method of the invention is used for controlling growth or infestation of fungus in or on a host cell or host organism, it is preferred that the nucleotide sequence does not share any significant homology with any host gene, or at least not with any essential gene of the host. In this context, it is preferred that the nucleotide sequence shows less than 60%, more preferably less than 50, 40 or 30% nucleic acid sequence identity with any gene of the host cell (when optimally aligned over the shortest sequence). If genomic sequence data, preferentially transcriptome data, is available for the host organism one may cross-check sequence identity with the nucleotide sequence using standard bioinformatics tools. In one embodiment, there is no sequence identity between the nucleotide sequence and the host sequences over 21 contiguous nucleotides, meaning that in this context, it is preferred that 21 contiguous base pairs of the nucleotide sequence do not occur in the genome of the host organism. In another embodiment, there is less than about 10% or less than about 12.5% sequence identity over 24 contiguous nucleotides of the nucleotide sequence with any nucleotide sequence from a host species.

The fungus to be controlled can be any organism belonging to the Kingdom Fungi. The methods of the invention are applicable to all fungi and fungal cells that are susceptible to gene silencing by RNA interference.

In one embodiment of the invention, the fungus may be a mould, or more particularly a filamentous fungus. In other embodiments of the invention, the fungus may be yeast.

In one embodiment the fungus may be a fungus belonging to the Phylum Basidiomycota.

In preferred, but non-limiting, embodiments of the invention the fungus is chosen from the group consisting of:

(1) a fungus of, or a cell derived from a plant pathogenic fungus, such as but not limited to Acremoniella spp., Alternaria spp. (e.g. Alternaria brassicola or Alternaria solani), Ascochyta spp. (e.g. Ascochyta pisi) Botrytis spp. (e.g. Botrytis cinerea or Botryotinia fuckeliana), Cladosporium spp., Cercospora spp. (e.g. Cercospora kikuchii or Cercospora zaea-maydis), Cladosporium spp. (e.g. Cladosporium fulvum), Colletotrichum spp. (e.g. Colletotrichum lindemuthianum), Curvularia spp., Diplodia spp. (e.g. Diplodia maydis), Erysiphe spp. (e.g. Erysiphe graminis f.sp. graminis, Erysiphe graminis f.sp. hordei or Erysiphe pisi) Erwinia armylovora, Fusarium spp. (e.g. Fusarium nivale, Fusarium sporotrichioides, Fusarium oxysporum, Fusarium graminearum, Fusarium germinearum, Fusarium culmorum, Fusarium solani, Fusarium moniliforme or Fusarium roseum), Gaeumanomyces spp. (e.g. Gaeumanomyces graminis f.sp. tritici), Gibberella spp. (e.g. Gibbera zeae), Helminthosporium spp. (e.g. Helminthosporium turcicum, Helminthosporium carbonum, Helminthosporium mavdis or Helminthosporium sigmoideum), Leptosphaeria salvinii, Macrophomina spp. (e.g. Macrophomina phaseolina), Magnaportha spp. (e.g. Magnaporthe oryzae), Mycosphaerella spp., Nectria spp. (e.g. Nectria heamatococca), Peronospora spp. (e.g. Peronospora manshurica or Peronospora tabacina), Phoma spp. (e.g. Phoma betae), Phakopsora spp. (e.g. Phakopsora pachyrhizi), Phymatotrichum spp. (e.g. Phymatotrichum omnivorum), Phytophthora spp. (e.g. Phytophthora cinnamomi, Phytophthora cactorum, Phytophthora phaseoli, Phytophthora parasitica, Phytophthora citrophthora, Phytophthora megasperma f.sp. soiae or Phytophthora infestans), Plasmopara spp. (e.g. Plasmopara viticola), Podosphaera spp. (e.g. Podosphaera leucotricha), Puccinia spp. (e.g. Puccinia sorghi, Puccinia striiformis, Puccinia graminis f.sp. tritici, Puccinia asparagi, Puccinia recondita or Puccinia arachidis), Pythium spp. (e.g. Pythium aphanidermatum), Pyrenophora spp. (e.g. Pyrenophora tritici-repentens or Pyrenophora teres), Pyricularia spp. (e.g. Pyricularia oryzae), Pythium spp. (e.g. Pythium ultimum), Rhincosporium secalis, Rhizoctonia spp. (e.g. Rhizoctonia solani, Rhizoctonia oryzae or Rhizoctonia cerealis), Rhizopus spp. (e.g. Rhizopus chinensid), Scerotium spp. (e.g. Scerotium rolfsii), Scierotinia spp. (e.g. Scierotinia scierotiorum), Septoria spp. (e.g. Septoria lycopersici, Septoria glycines, Septoria nodorum or Septoria tritici), Thielaviopsis spp. (e.g. Thielaviopsis basicola), Tilletia spp., Trichoderma spp. (e.g. Trichoderma virde), Uncinula spp. (e.g. Uncinula necator), Ustilago maydis (e.g. corn smut), Venturia spp. (e.g. Venturia inaequalis or Venturia pirina) or Verticillium spp. (e.g. Verticillium dahliae or Verticillium albo-atrum);

(2) a fungus of, or a cell derived from a fungus capable of infesting humans such as, but not limited to, Candida spp., particularly Candida albicans; Dermatophytes including Epidermophyton spp., Trichophyton spp, and Microsporum spp.; Aspergillus spp. (particularly Aspergillus flavus, Aspergillus fumigatus, Aspergillus nidulans, Aspergillus niger or Aspergillus terreus); Blastomyces dermatitidis; Paracoccidioides brasiliensis; Coccidioides immitis; Cryptococcus neoformans; Histoplasma capsulatum Var. capsulatum or Var. duboisii; Sporothrix schenckii; Fusarium spp.; Scopulariopsis brevicaulis; Fonsecaea spp.; Penicillium spp.; or Zygomycetes group of fungi (particularly Absidia corymbifera, Rhizomucor pusillus or Rhizopus arrhizus);

(3) a fungus of, or a cell derived from a fungus capable of infesting animals such as, but not limited to Candida spp., Microsporum spp. (particularly Microsporum canis or Microsporum gypseum), Trichophyton mentagrophytes, Aspergillus spp., or Cryptococcus neoforman;

and

(4) a fungus of, or a cell derived from a fungus that causes unwanted damage to substrates or materials, such as fungi that attack foodstuffs, seeds, tuber(s), wood, paint, plastic, clothing etc. Examples of such fungi are the moulds, including but not limited to Stachybotrys spp., Aspergillus spp., Alternaria spp., Cladosporium spp., Penicillium spp. or Phanerochaete chrysosporium.

Preferred plant pathogenic fungi to be controlled according to the invention are Cercospora spp. (e.g. Cercospora kikuchii or Cercospora zaea-maydis) causing e.g. black and yellow sigatoka in banana; Colletotrichum spp. (e.g. Colletotrichum lindemuthianum) causing e.g. anthracnose in corn; Curvularia spp. causing seedling blight; Diplodia spp. (e.g. Diplodia maydis) causing e.g. ear, kernel and stalk rots in corn; Fusarium spp. (e.g. Fusarium nivale, Fusarium oxysporum, Fusarium graminearum, Fusarium germinearum, Fusarium culmorum, Fusarium solani, Fusarium moniliforme or Fusarium roseum) causing e.g. ear, kernel and stalk rots in corn, fusarium wilt in cotton and Panama disease in banana; Gibberella spp. causing e.g. ear, kernel and stalk rots in corn; Magnaportha spp. (e.g. Magnaporthe oryzae) causing rice blast; Mycosphaerella spp. causing e.g. black and yellow sigatoka in banana; Phakopsora spp. (e.g. Phakopsora pachyrhizi) causing e.g. soybean rust; Phytophthora spp.(e.g. Phytophthora cinnamomi, Phytophthora cactorum, Phytophthora phaseoli, Phytophthora parasitica, Phytophthora citrophthora, Phytophthora megasperma f.sp. soiae or Phytophthora infestans) causing e.g. late blight in potato and tomato; Puccinia spp. (e.g. Puccinia sorghi, Puccinia striiformis (yellow rust), Puccinia graminis f.sp. tritici, Puccinia asparagi, Puccinia recondita or Puccinia arachidis) causing e.g. common rust in corn; Rhizoctonia spp. (e.g. Rhizoctonia solani, Rhizoctonia oryzae or Rhizoctonia cerealis) causing e.g. sheath blight in rice or early blight in potato; Rhizopus spp. (e.g. Rhizopus chinensid) causing seedling blight; Trichoderma spp. (e.g. Trichoderma virde) causing seedling blight; or Verticillium spp. (e.g. Verticillium dahliae or Verticillium albo-atrum) causing e.g. verticillium wilt in cotton.

Particularly preferred plant pathogenic fungi according to the invention are Rhizoctonia spp. (e.g. Rhizoctonia solani, Rhizoctonia oryzae or Rhizoctonia cerealis) causing e.g. sheath blight in rice, causing black scurf, wilts or rots in potato or causing Rhizoctonia blight on turfgrasses.

In particular, the disclosure relates to methods, wherein the pathogenic Rhizoctonia spp. is a rice or a potato pathogenic Rhizoctonia spp.

Thus, the present invention extends to methods as described herein, wherein the pathogenic fungus is a Rhizoctonia strain (e.g. Rhizoctonia solani, Rhizoctonia oryzae or Rhizoctonia cerealis). Preferably, the Rhizoctonia strain is chosen from the group comprising Rhizoctonia solani, e.g. Rhizoctonia solani ZG3, anastomosis group AG11, Rhizoctonia solani ZG5, anastomosis group AG2-1 or rice infecting Rhizoctonia solani, anastomosis group AG1-1A.

The concept of anastomosis groups is used to characterize and identify Rhizoctonia. The concept implies that isolates of Rhizoctonia that have the ability to recognize and fuse (i.e. “anastomose”) with each other are genetically related, whereas isolates of Rhizoctonia that do not have this ability are genetically unrelated.

In one specific embodiment the plant is lupine or wheat and the pathogenic fungal target gene is a gene from the group of fungi comprising Rhizoctonia solani ZG3.

In another specific embodiment the plant is canola or a crucifer and the pathogenic fungal target gene is a gene from the group of fungi comprising Rhizoctonia solani ZG5.

In a further specific embodiment the plant is rice, corn, sorghum or soybean and the pathogenic fungal target gene is a gene from the group of fungi comprising rice infecting Rhizoctonia solani AG1-1A.

In another specific embodiment the plant is potato and the pathogenic fungal target gene is a gene from the group of fungi comprising Rhizoctonia solani, anastomosis groups AG3, AG4, AG5 and AG9.

The fungus may be an intact fungal cell, meaning that the fungal cell has a cell wall. In this non-limiting embodiment, the intact fungal cell is contacted with the nucleotide sequence as described herein; meaning that the cell wall of the fungal cell need not be removed prior to contact with the nucleotide sequence.

As used herein the term “fungus” encompasses the fungus as such and also fungal cells of all types and at all stages of development, including specialised reproductive cells such as sexual and asexual spores, and also other life forms of the fungus, such as haustoria, conidia, sclerotia, mycelium, penetration peg, spore, zoospores etc.

In cases where fungi reproduce both sexually and asexually, these fungi have two names: the teleomorph name describes the fungus when reproducing sexually; the anamorph name refers to the fungus when reproducing asexually. The holomorph name refers to the “whole fungus”, encompassing both reproduction methods.

According to one embodiment of the present invention, the fungal cell which is contacted with the nucleotide sequence is a plant pathogenic fungal cell in a life stage outside a plant cell, for example in the form of a hypha, germ tube, appressorium, sclerotium, conidium (asexual spore), ascocarp, cleistothecium, or ascospore (sexual spore outside the plant).

According to another embodiment of the present invention, the fungal cell which is contacted with the nucleotide sequence is a plant pathogenic fungal cell in a life stage inside a plant cell, for example a pathogenic form such as a penetration peg, a hypha, a spore or a haustorium.

In another embodiment the present invention extends to methods as described herein, wherein the pathogenic fungus is a plant pathogenic fungus such as a rice, a potato or a turfgrass pathogenic fungus.

In other embodiments the pathogenic fungus, plant, part of a plant, reproductive plant material, plant seed, tuber(s), substrate or material may be provided with a composition containing any of the nucleotide sequences as described herein, preferably containing any of the ribonucleotide sequences or ribonucleotide molecules or dsRNA's as described herein. The composition may, in addition to the nucleotide sequence, contain one or more further excipients, diluents or carriers, preferably at least 1, 2 or 3 excipients, diluents or carriers. In another embodiment, the pathogenic fungus, plant, part of a plant, reproductive plant material, plant seed, tuber(s), substrate or material may be provided with a composition containing more than one of the nucleotide sequences, preferably ribonucleotide sequences, described herein.

In other embodiments the pathogenic fungus, plant, part of a plant, reproductive plant material, plant seed, tuber(s), substrate or material may be provided with a composition containing the host cell or host organism expressing any of the nucleotide sequences or ribonucleotide sequences or dsRNA's described herein. The composition may, in addition to the host cell or host organism, contain one or more further excipients, diluents or carriers, preferably at least 1, 2 or 3 excipients, diluents or carriers. In another embodiment, the pathogenic fungus, plant, part of a plant, reproductive plant material, plant seed, tuber(s), substrate or material may be provided with a composition containing more than one host cell or host organism each expressing one or more of the nucleotide or ribonucleotide sequences described herein.

Also encompassed are the methods described herein, wherein said pathogenic fungus, plant, part of a plant, reproductive plant material, plant seed, tuber(s), substrate or material is contacted with more than one of the herein described nucleotide sequences or ribonucleotide sequences or dsRNA's, either simultaneously or sequentially provided.

Further encompassed are the methods described herein, wherein said pathogenic fungus, plant, part of a plant, reproductive plant material, plant seed, tuber(s), substrate or material is contacted with more than one of the herein described host cells or host organisms expressing said nucleotide sequence or ribonucleotide sequence or dsRNA, either simultaneously or sequentially provided.

According to another embodiment, the methods of the invention rely on a GMO approach wherein said nucleotide sequence is expressed by a cell or an organism infested with or susceptible to infestation by fungi. Preferably, said cell is a plant cell or said organism is a plant. Therefore, in a preferred embodiment of the invention the nucleotide sequence may be expressed by (e.g. transcribed within) a host cell or host organism, the host cell or organism being an organism susceptible or vulnerable to infestation with a fungus. In this embodiment RNAi-mediated gene silencing of one or more target genes in the fungus may be used as a mechanism to control growth of the fungus in or on the host cell or organism and/or to prevent or reduce fungal infestation of the host cell or organism. Thus, expression of the nucleotide sequence within cells of the host organism may confer resistance to a particular fungus or to a class of fungi. In case the nucleotide sequence inhibits more than one fungal target gene, expression of the nucleotide sequence within cells of the host organism may confer resistance to a fungus at distinct stages of its development, or alternatively may confer resistance to more than one fungus or more than one class of fungi when non-conservative target genes are chosen for inhibition.

In a preferred embodiment the host organism is a plant and the fungus is a plant pathogenic fungus. In this embodiment the fungal cell is contacted with the nucleotide sequence by expressing the nucleotide sequence in a plant or plant cell that is infested with or susceptible to infestation with the plant pathogenic fungus.

In this context the term “plant” encompasses any plant material that it is desired to be treated to prevent or reduce fungal growth and/or fungal infestation. This includes, inter alia, whole plants, seedlings, propagation or reproductive plant material such as seeds, tuber(s), cuttings, grafts, explants, etc. and also plant cell and tissue cultures. The plant material should express, or have the capability to express, a nucleotide sequence, preferably a ribonucleotide sequence, that inhibits one or more target genes of the fungus.

Therefore, in a further aspect the invention provides a plant, preferably a transgenic plant, or propagation or reproductive plant material for a (transgenic) plant, or a plant cell culture expressing or capable of expressing at least one nucleotide sequence of the present invention, wherein the nucleotide sequence comprises at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120 or 150 contiguous nucleotides that are complementary to a fungal target gene, such that the nucleotide sequence is taken up by a fungal cell upon plant-fungus interaction, said nucleotide sequence being capable of inhibiting the target gene or down-regulating expression of the target gene by RNA interference. The target gene may be any of the target genes herein described, for instance a target gene that is essential for the viability, growth, development or reproduction of the fungus, preferably said fungal target gene is involved in any of the cellular functions as defined earlier. In this embodiment the fungal cell can be any fungal cell, but is preferably a fungal cell of a plant pathogenic fungus. Preferred plant pathogenic fungi include, but are not limited to, Rhizoctonia spp.

A plant to be used in the methods of the invention, or a transgenic plant according to the invention encompasses any plant, but is preferably a plant that is susceptible to infestation by a plant pathogenic fungus, including but not limited to the following plants: rice, potato, corn, soybean, cotton, banana, tomato, vine, apple, pear, sorghum, millet, beans, groundnuts, rapeseed, sunflower, sugarcane, sugar beet, tobacco, onion, peanuts and cereals including wheat, oats, barley, rye and turfgrass. Most preferably the plant is rice, potato, turfgrass, corn, soybean, cotton, banana or tomato.

Accordingly, the present invention also extends to methods as described herein wherein the plant is one of the plants mentioned earlier, preferably rice, potato, turfgrass, corn, soybean, cotton, banana or tomato.

In one embodiment the transgenic plant or plant cell comprises at least one nucleic acid molecule described herein, at least one expression cassette described herein or at least one dsRNA molecule described herein. The transgenic plant or plant cell is preferably rice, potato or turfgrass.

In an alternative embodiment the transgenic plant or plant cell according to the invention comprises more than one nucleic acid molecule, more than one expression cassette or more than one dsRNA molecule described herein. As a non-limiting example, the transgenic plant or plant cell comprises for instance 2, 3, 4 or 5 expression cassettes, wherein each expression cassette is capable of expressing a different RNA molecule, preferably a dsRNA, containing a nucleotide sequence identical to at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120 or 150 contiguous nucleotides of a pathogenic fungal target gene as described herein. As such the transgenic plant is capable of silencing more than one pathogenic fungal target gene, for instance 2, 3, 4 or 5 target genes, or is capable of targeting more than one target region, for instance 2, 3, 4 or 5 target regions, wherein a target region is a region of at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120 or 150 contiguous nucleotides of a pathogenic fungal target gene as described herein.

In another embodiment the transgenic plant or plant cell according to the invention is a plant or plant cell as described above,
(i) wherein the nucleic acid molecule(s) or the expression cassette(s) comprise(s) more than one nucleic acid molecule having a nucleotide sequence corresponding to (or being similar in sequence to such a degree that when the two sequences are optimally aligned and compared using the BLASTN alignment tool or using a global alignment based on approximate string matching as described herein, said nucleic acid molecule is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to) more than one target gene, or
(ii) wherein the at least one dsRNA molecule(s) is (are) produced from the expression of a nucleic acid molecule or an expression cassette that comprises more than one nucleotide sequence corresponding to (or being similar in sequence to such a degree that when the two sequences are optimally aligned and compared using the BLASTN alignment tool or using a global alignment based on approximate string matching as described herein, said nucleic acid molecule is at least 70, 71, 72, 73, 74, preferably 75, 76, 77, 78, 79, 80, more preferably 81, 82, 83, 84, 85, 86, 87, 88, 89, 90, 91, 92, 93, 94, 95, 96, 97, 98 or 99% identical to) more than one target gene, wherein the more than one target gene can be a target gene from the same pathogenic Rhizoctonia strain or from different pathogenic Rhizoctonia strains. This further aspect of the invention allows increasing the efficacy of the control of the fungus' viability, growth and/or development.

In the embodiments described herein the more than one nucleotide sequence can be combined as several short fragments in one longer RNA molecule, preferably a dsRNA molecule, thus the RNA molecule containing more than one nucleotide sequence, each nucleotide sequence identical to at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120 or 150 contiguous nucleotides of a fungal target gene as described herein. Alternatively, the more than one nucleotide sequence each form a separate RNA molecule, preferably a dsRNA molecule, containing said nucleotide sequence identical to at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120 or 150 contiguous nucleotides of a fungal target gene as described herein.

Transgenic plants according to the invention extend to all plant species described herein being resistant to the respective fungus species as specifically described herein.

According to still other embodiments of the invention the transgenic plant or plant cell is rice, potato or turfgrass, wherein the more than one target gene are genes from a rice pathogenic fungus, a potato pathogenic fungus and/or a turf grass pathogenic fungus, more preferably a gene from a fungus selected from the group comprising Rhizoctonia spp., Magnaporthe spp., Fusarium spp., Acremoniella spp., Pythium spp., Curvularia spp., Trichoderma spp. and Rhizopus spp. In a preferred embodiment, the more than one target genes are genes from either the same or different Rhizoctonia strain(s).

The transgenic plants or plant cells as described herein are resistant to one or more (plant) pathogenic fungi, wherein the fungi can be chosen from the phylum of the Basidiomycota or can be chosen from Rhizoctonia spp., e.g. Rhizoctonia solani. (e.g. the group comprising Rhizoctonia solani ZG3, anastomosis group AG11, the group comprising Rhizoctonia solani ZG5, anastomosis group AG2-1 or the group comprising the rice infecting Rhizoctonia solani, anastomosis group AG1-1A).

The fungal target gene may be any target gene herein described. Preferably the regulatory sequence(s) in the expression cassette is (are) a regulatory sequence(s) that is (are) active in a plant cell. More preferably, the regulatory sequence(s) is (are) originating from a plant. Preferably, the regulatory sequence is selected from the group comprising constitutive promoters or tissue specific promoters as described later herein. Encompassed by the aforementioned term “regulatory sequence” are promoters and nucleic acids or synthetic fusion molecules or derivatives thereof which activate or enhance expression of a nucleic acid molecule, so called activators or enhancers. The expression “directs the expression” as used herein refers to a functional linkage between the regulatory sequence and the nucleic acid molecule of interest, such that the regulatory sequence is able to initiate transcription of the nucleic acid molecule of interest. The term “regulatory sequence” is to be taken in a broad context and refer to a regulatory nucleic acid capable of effecting expression of the nucleic acid molecule(s) to which it is operably linked.

By way of example, the nucleic acid molecule encoding the RNA molecule, preferably dsRNA, could be placed under the control of an inducible or growth-specific or developmental stage-specific promoter which permits transcription of the RNA molecule, preferably dsRNA, to be turned on, by the addition of the inducer for an inducible promoter or when the particular stage of growth or development is reached.

Alternatively, the nucleic acid molecule encoding the RNA molecule, preferably a dsRNA, is placed under the control of a strong constitutive promoter such as any selected from the group comprising the ubiquitin promoter, CaMV35S promoter, doubled CaMV35S promoter, actin promoter, GOS2 promoter and Figwort mosaic viruse (FMV) 34S promoter.

In order to improve the transfer of the RNA molecule, preferably dsRNA, from the plant cell to the plant pest, the plants could preferably express the nucleic acid molecule or expression cassette in a plant part that is first accessed or damaged by the plant pest. In case of a plant pathogenic fungi, preferred tissues to express the RNA molecule are the roots, leaves and stem. Therefore, in the methods of the present invention, a plant tissue-preferred promoter may be used, such as a root specific promoter, a leaf specific promoter or a stem-specific promoter. Examples of tissue-specific promoters are root specific promoters of genes encoding PsMTA Class III Chitinase, photosynthetic tissue-specific promoters such as the rubisco promoter and the mPEPC promoter. Other such promoters are promoters of cab1 and cab2, rbcS, gapA, gapB and ST-LS1 proteins, JAS promoters, chalcone synthase promoter and promoter of RJ39 from strawberry.

In yet other embodiments of the present invention, other promoters useful for the expression of the RNA molecule, preferably dsRNA, are used and include, but are not limited to, promoters from an RNA PolI, an RNA PolII, an RNA PolIII, T7 RNA polymerase or SP6 RNA polymerase. These promoters are typically used for in vitro-production of the RNA molecule. These promoters can also be used for in vivo-production of the RNA molecule in a host cell, such as a bacterial cell as described earlier. The (ds)RNA molecule as such, or the host cells producing the (ds)RNA molecule is then included in an antifungal agent, for example in an anti-fungal liquid, spray or powder.

The present invention also encompasses a method for generating any of the RNA molecules of the invention. This method comprises the steps of (a) contacting an isolated nucleic acid molecule or an expression cassette of the invention with cell-free components; or (b) introducing (e.g. by transformation, transfection or injection) an isolated nucleic acid molecule or an expression cassette of the invention in a cell, under conditions that allow transcription of said nucleotide sequence or expression cassette to produce the RNA molecule, preferably dsRNA.

Optionally, one or more transcription termination sequences may also be incorporated in the expression cassette of the invention. The term “transcription termination sequence” encompasses a control sequence at the end of a transcriptional unit, which signals 3′ processing and poly-adenylation of a primary transcript and termination of transcription. Additional regulatory elements, such as transcriptional or translational enhancers, may be incorporated in the expression cassette.

The expression cassettes of the invention may further include an origin of replication which is required for maintenance and/or replication in a specific cell type. One example is when an expression cassette is required to be maintained in a bacterial cell as an episomal genetic element (e.g. plasmid or cosmid molecule) in a cell. Preferred origins of replication include, but are not limited to, f1-ori and colE1-ori.

The expression cassette may optionally comprise a selectable marker gene. As used herein, the term “selectable marker gene” includes any gene, which confers a phenotype on a cell in which it is expressed to facilitate the identification and/or selection of cells, which are transfected or transformed with an expression cassette of the invention. Examples of suitable selectable markers include resistance genes against kanamycin (Kanr), hygromycin, Bar, ampicillin (Ampr), tetracycline (Tcr), phosphinothricin, and chloramphenicol (CAT) gene. Other suitable marker genes provide a metabolic trait, for example manA. Visual marker genes may also be used and include for example beta-glucuronidase (GUS), luciferase and Green Fluorescent Protein (GFP).

Plants that have been stably transformed with a nucleic acid molecule or an expression cassette encoding the RNA molecule, preferably dsRNA, may be supplied as seed, reproductive plant material, propagation material or cell culture material which does not actively express the RNA molecule, preferably dsRNA, but has the capability to do so.

In order to express an RNA molecule as described herein in plants for the purposes of down-regulating expression of a target gene in a plant pathogenic fungus it may be necessary only for the plant to express (transcribe) the RNA molecule in a part of the plant which will come into direct contact with the fungus, such that the RNA molecule can be taken up by the fungus. Depending on the nature of the fungus and its relationship with the host plant, expression of the RNA molecule could occur within a cell or tissue of a plant within which the fungus is also present during its life cycle, or the RNA molecule may be secreted into a space between cells, such as the apoplast, that is occupied by the fungus during its life cycle. Furthermore, the RNA molecule may be located in the plant cell, for example in the cytosol, or in the plant cell organelles such as a chloroplast, mitochondrion, vacuole or endoplastic reticulum.

Alternatively, the RNA molecule may be secreted by the plant cell and by the plant to the exterior of the plant. As such, the RNA molecule may form a protective layer on the surface of the plant.

The invention also relates to methods of making a transgenic plant or plant cell as herein described, capable of expressing (or expressing) an RNA molecule, preferably at least one dsRNA comprising annealed complementary strands, one of which comprises at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120, 150, 200, 300, 400, 500, 600, 700, 800, 900 or 1000 contiguous nucleotides identical to a target gene in a pathogenic Rhizoctonia spp., said method comprising the steps of:
a) providing a nucleic acid molecule described herein, wherein the nucleic acid molecule is able to form (or is forming) at least one RNA molecule once expressed in the plant; or providing an expression cassette described herein, wherein the at least one nucleic acid molecule expressed from said expression cassette is able to form (or is forming) at least one RNA molecule, preferably at least one dsRNA, once expressed in the plant;
b) transforming a recipient plant with said nucleic acid molecule or with said expression cassette;
c) producing one or more offspring of said recipient plant; and
d) testing the offspring for expression of said RNA molecule.

The invention also relates to methods of producing a plant that is resistant to Rhizoctonia spp. infection comprising:
(a) crossing a transgenic plant as described herein with another plant, and
(b) selecting Rhizoctonia-resistant progeny by analyzing for the presence of a nucleotide sequence that encodes at least one dsRNA comprising annealed complementary strands, one of which comprises at least 17, preferably at least 18, 19, 20 or 21, more preferably at least 22, 23, 24, 25, 26, 27, 28, 29, 30, 31, 32, 33, 34, 35, 40, 45, 50, 60, 70, 80, 90, 100, 110, 120, 150, 200, 300, 400, 500, 600, 700, 800, 900 or 1000 contiguous nucleotides identical to at least one sequence selected from the group consisting of any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof.

The invention also relates to methods described herein further comprising the step of backcrossing the progeny plant that is fungus resistant to the second parent plant, and further selecting for fungus-resistant progeny by analyzing for the presence of at least one nucleotide sequence selected from the group consisting of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof.

The invention also relates to methods of producing a fungus-resistant plant comprising:
(a) sexually crossing a first fungus-resistant plant as described herein with a second parent plant that lacks the tolerance to fungal infection, thereby producing a plurality of progeny plants;
(b) selecting a first progeny plant that is resistant to fungi,
(c) selfing said first progeny plant, thereby producing a plurality of second progeny plants;
(d) repeated selfing of said progeny for 1, 2, 3, 4, or 5 more generations; and
(d) selecting from said second progeny plants a plant that is resistant to fungi.

The invention also relates to methods of producing a fungus-resistant plant comprising:
(a) sexually crossing a first fungus-resistant plant as described herein with a second parent plant that lacks the tolerance to fungi, thereby producing a plurality of progeny plants; and
(b) selecting a first progeny plant that is resistant to fungi, and
(c) backcrossing the progeny plant that is fungus-resistant to the second parent plant and further selecting for fungus-resistant progeny.

The invention also relates to methods as described hereabove, wherein the fungus-resistant progeny of step (c) is detectable by the presence of a nucleotide sequence comprising any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or complements thereof.

Also encompassed herein are transgenic plants or transgenic plant cells obtained by the methods described herein; and the seed or tuber(s) produced from these (transgenic) plants. In a specific embodiment, the plant is chosen from rice, potato or turfgrass.

The present disclosure further relates to hybrid seed or transgenic tuber(s) produced by crossing a first inbred plant with a second, distinct inbred plant wherein the first or second inbred plant is a transgenic plant as described herein, and wherein the plant is chosen from rice, potato or turfgrass.

The disclosure encompasses a method for producing hybrid seed or transgenic tuber(s) as described herein, wherein crossing comprises the steps of:
(a) planting the seeds or tuber(s) of first and second inbred plants;
(b) cultivating the seeds or tuber(s) of said first and second inbred plants into plants that bear flowers;
(c) preventing self pollination of at least one of the first or second inbred plant;
(d) allowing cross-pollination to occur between the first and second inbred plants; and
(e) harvesting seeds or tuber(s) on at least one of the first or second inbred plants, said seeds or tuber(s) resulting from said cross-pollination;
   wherein the plant is chosen from rice, potato or turfgrass.

Also encompassed herein are hybrid plants produced by growing the seed or tuber(s) described herein, wherein the plant is chosen from rice, potato or turfgrass

In a further embodiment, the disclosure relates to a composition for preventing and/or controlling fungal growth and/or fungal infestation, wherein the composition may be in any suitable physical form for application or exposure to any pathogenic fungus, plant, part of a plant, reproductive plant material, plant seed, tuber(s), substrate or material described herein.

The present disclosure further relates to any nucleotide sequence, ribonucleotide sequence, host cell, host organism, nucleic acid molecule, expression cassette, dsRNA molecule, transgenic plant cell or composition as described herein for use as a medicine. The present invention also relates to any nucleotide sequence, ribonucleotide sequence, host cell, host organism, nucleic acid molecule, expression cassette, dsRNA molecule, transgenic plant cell or composition as described herein for the treatment of a fungal disease or of a fungal infection of humans or animals, such as mycosis.

The composition may be a composition suitable for topical use, such as application on the skin of an animal or human, for example as liquid composition to be applied to the skin as drops, gel, aerosol, or by brushing, or a spray, cream, ointment, etc. for topical application or as transdermal patches.

Alternatively, the at least one nucleotide sequence, ribonucleotide sequence, host cell, host organism, nucleic acid molecule, expression cassette, dsRNA molecule, transgenic plant cell or composition described herein is produced by bacteria (e.g. lactobacillus) which can be included in food and which functions as an oral vaccine against the fungal infection.

Other conventional pharmaceutical dosage forms may also be produced, including tablets, capsules, pessaries, transdermal patches, suppositories, etc. The chosen form will depend upon the nature of the target fungus and hence the nature of the disease it is desired to treat.

In one specific embodiment, the composition may be a coating that can be applied to a substrate or material in order to protect the substrate or material from infestation by a fungus and/or to prevent, arrest or reduce fungal growth on the substrate or material and thereby prevent damage caused by the fungus. In this embodiment, the composition can be used to protect any substrate or material that is susceptible to infestation by or damage caused by a fungus, for example foodstuffs and other perishable materials, and substrates such as wood. Preferred target fungal species for this embodiment include, but are not limited to, the following: Stachybotrys spp., Aspergillus spp., Alternaria spp. or Cladosporium spp.

The present invention further encompasses a method for treating and/or preventing fungal infestation on a substrate or material comprising applying an effective amount of any of the compositions described herein to said substrate or material.

In another embodiment of the invention the compositions are used as a fungicide for a plant or for propagation or reproductive plant material of a plant, such as on seeds. As an example, the composition can be used as a fungicide by spraying or applying it on plant tissue or spraying or mixing it on the soil before or after emergence of the plantlets.

In another embodiment the invention relates to the use of any nucleotide sequence, ribonucleotide sequence, host cell, host organism, nucleic acid molecule, expression cassette, dsRNA molecule, transgenic plant cell or composition as described herein for preventing fungal infestation of plants susceptible to fungal infection; or for treating fungal infection of plants. Specific plants to be treated for fungal infections caused by specific fungal species are as described earlier and are encompassed by the said use.

According to a still further embodiment, the present invention extends to a method for increasing plant yield in the presence of a fungus infestation comprising introducing in a plant any of the nucleic acid molecules or expression cassettes as herein described. Plants encompassed by this method are as described earlier. Preferably, said plant is rice, potato or turfgrass.

The invention will be further understood with reference to the following non-limiting examples.

### EXAMPLES

### Example 1

### Degenerate Primer Design

A blast search was performed for each target by blasting the aminoacid sequence of Magnaporthe against non redundant protein databases of different fungi. A selection of protein sequences was made from the hits (Gibberella zeae, Scierotinia scierotiorum, Aspergillus niger, Neosartorya fischeri and Candida albicans). The orthology between the sequences was confirmed by reverse blastp analysis against the Magnaporthe protein database.

These sequences were submitted to a “Block maker” program (http://blocks.fhcrc.org/make_blocks.html) which produced multiple sequence alignments and analyses of the sequences for regions of conservation.

Blocks of conserved amino acid sequences were submitted to CodeHop (http://blocks.fhcrc.org/codehop.html). From the output of degenerate primers a selection of approximately 10 forward and 10 backward primers for each target was made.

### Example 2

### Amplification of Target Genes

a) Amplification of Target Genes from Rhizoctonia solani (ZG3, AG11)

Total RNA was prepared from the fungus Rhizoctonia solani ZG3, anastomosis group AG11. A 6 day old fungus culture on potato dextrose (PD) agar was used to inoculate a liquid culture of 200 ml PD broth. After 4 (ZG3 and rice infecting strain) respectively 7 (ZG5) days of incubation (28° C., gently shaken) of incubation, the mycelium was harvested by filtration through 4 layers of miracloth, washed and dried.

Total RNA was prepared using the RNeasy Mini kit for plants (QIAGEN Cat. No. 74904). Traces of genomic DNA were removed by on-column DNase digestion using the RNase-Free DNase set (QUIAGEN Cat. No. 79254) as well as an additional incubation with RQ1 DNase (PROMEGA Cat. No M610A) according to manufacturers manual. The quality of DNA free RNA was tested by PCR, using R. solani β-tubulin specific primers (SEQ ID NOs 251 and 252).

A reverse transcription reaction (Superscript III, INVITROGEN) was done with on the total RNA to produce the first strand cDNA. The quality of the cDNA was tested by PCR, using R. solani β-tubulin specific primers (SEQ ID NOs 251 and 252). The cDNA was then used to amplify the R. solani target genes by degenerate family PCR.

For degenerate family PCR on R. solani ZG3 only those primer combinations were chosen which gave a calculated fragment between 200 and 2000 bp.

Conditions for the degenerate family PCR were as follows for targets 13, 16, 21, 27 and 28: AmpliTaq Gold PCR system (Applied Biosystems), using 1 μ1 of cDNA in 20 μ1 of reaction mix, 10′ 95° C., 10 cycles (30″ 95° C., 30″ 55° C. (touchdown, 0.5° C. per cycle), 2′ 72° C.), 33 cycles (30″ 95° C., 30″ 50° C., 2′ 72° C.), 7′ 72° C. These PCR conditions were used for all PCRs if not noted otherwise.

The cDNA's obtained by degenerate family PCR were purified from the gel by a gel extraction kit (QUIAGEN Cat. No. 28706) and cloned into a TOPO TA vector (Invitrogen). Ideally the target sequence was amplified by 2-3 different primer combinations and for each primer combination at least three clones were sequenced. The consensus sequence was blasted against the Magnaporthe grisea protein database form the Broad Institute of MIT and Harvard, Cambridge.

For targets 13, 21 and 28, two target sequences were found, one upstream and one downstream. For target 13, a specific forward (SEQ ID NO 77) and backward (SEQ ID NO 78) primer was designed to combine the two overlapping target sequences by PCR. The two sequences for target 21 were estimated to be separated by 70 aminoacids. The sequence in-between the two amplified regions was not amplified. To amplify the target sequence in between the two sequences found for target 28, a specific backward (SEQ ID NO 250) primer was designed.

For targets 1, 6 and 15 a nested PCR was done. A first PCR was performed with selected degenerate primer combination, using the previous described PCR conditions with increased cDNA volume (5 μl). 1 μl of this first PCR was subjected to a second round of amplification under the same conditions using selected inner primers. Fragments of consecutive sizes were isolated, cloned and sequenced as described. Upon positive identification by blast analysis specific primers were designed for each target to amplify the target sequence in a first step PCR with increased (5 μl) cDNA volume and increased cycles (42×). This approach was only successful for target 1 using the specific primers (SEQ ID NO 21 and 22). For each primer combination at least three clones were sequenced. The consensus sequence was blasted against the Magnaporthe grisea protein database form the Broad Institute of MIT and Harvard, Cambridge.

The degenerate and specific primer combinations which successfully amplified the target sequences from R. solani ZG3 are represented in Table 1. The target sequences from R. solani ZG3 are represented in Table 2.

Upon positive blast analysis of the target sequences, selected TOPO clones were used for dsRNA hairpin construction.

b) Amplification of Target Sequences from Rhizoctonia solani ZG5

Total RNA was prepared from Rhizoctonia solani ZG5 (anastomosis group AG2-1).

Culturing of the fungus, RNA and cDNA isolation and target amplification were done as described for Rhizoctonia solani ZG3 in a).

For the target amplification, the degenerate or specific primer combinations which also successfully amplified the target sequences from R. solani ZG3, were used (see Table 1).

PCR conditions conditions for the degenerate family PCR were as follows for targets 1, 13, 15, 16, 21, and 28 for R. solani ZG5: AmpliTaq Gold PCR system (Applied Biosystems), using 2 μl of cDNA in 20 μl of reaction mix, 10′ 95° C., 10 cycles (30″ 95° C., 30″ 55° C. (touchdown, 0.5° C. per cycle), 2′ 72° C.), 33 cycles (30″ 95° C., 30″ 50° C., 2′ 72° C.), 7′ 72° C. For target 6 a nested PCR was tried but was unsuccessful. Target 27 could not be amplified.

The cDNA's obtained by degenerate family PCR were purified from the gel by a gel extraction kit (QUIAGEN Cat. No. 28706) and cloned into a TOPO TA vector (Invitrogen). For each primer combination at least three clones were sequenced. The consensus sequence was blasted against the Magnaporthe grisea protein database form the Broad Institute of MIT and Harvard, Cambridge.

For target 21 two target sequences were found, one upstream and one downstream. The two sequences for target 21 were estimated to be separated by 70 aminoacids. The sequence in-between the two amplified regions was not amplified.

The target sequences from R. solani ZG5 are represented in Table 3.

c) Amplification of Target Sequences from Rice Infecting Rhizoctonia solani

Total RNA was prepared from a rice infecting Rhizoctonia solani strain (anastomosis group AG1-1A).

Culturing of the fungus, RNA and cDNA isolation and target amplification were done as described under a).

For the target amplification, the degenerate or specific primer combinations which also successfully amplified the target sequences from R. solani ZG3, were used (see Table 1).

PCR conditions conditions for the degenerate family PCR were as follows for targets 1, 13, 15, 16, 21, 27 and 28 for the rice infecting strain: AmpliTaq Gold PCR system (Applied Biosystems), using 2 μl of cDNA in 20 μl of reaction mix, 10′ 95° C., 10 cycles (30″ 95° C., 30″ 55° C. (touchdown, 0.5° C. per cycle), 2′ 72° C.), 33 cycles (30″ 95° C., 30″ 50° C., 2′ 72° C.), 7′ 72° C. For target 6 a nested PCR was tried but was unsuccessful.

The cDNA's obtained by degenerate family PCR were purified from the gel by a gel extraction kit (QUIAGEN Cat. No. 28706) and cloned into a TOPO TA vector (Invitrogen). For each primer combination at least three clones were sequenced. The consensus sequence was blasted against the Magnaporthe grisea protein database form the Broad Institute of MIT and Harvard, Cambridge.

The target sequences from the rice infecting Rhizoctonia solani strain are represented in Table 4.

### Example 3

### Selection and Cloning of Target Nucleotide Sequences of the Target Genes of Rhizoctonia solani for RNAi Mediated Gene Silencing

Fragments of the target genes (see Table 2) were selected for further in vivo RNAi experiments and were cloned in a hairpin construct to produce dsRNA in a plant cell.

For R. solani ZG3, AG11, for targets 6, 13, 15, 16, 27 and 28, a 400 bp fragment (respectively SEQ ID NO 25, 52, 81, 104, 183 and 217); for target 1, a 300 bp fragment (SEQ ID NO 7); and for target 21, a 401 bp fragment (SEQ ID NO 148) was chosen to be cloned by gateway technology (Invitrogen) in an antisense-sense orientation into the plant expression vector pK7GWIWG2D(II) (received under MTA from the Vlaams lnstituut voor Biotechnologie (VIB), Belgium; Karimi M., Inze D., Depicker A., Gateway vectors for Agrobacterium-mediated plant transformation, Trends Plant Sci. 2002 May;7(5): 193-195). Target specific forward and backward primers were designed which added the DNA recombination sequences (att sites) to the target fragment by PCR amplification (see Table 5).

The resulting hairpin sequences for targets 1, 6, 13, 15, 16, 21, 27 and 28 are represented respectively by SEQ ID NOs 8, 26, 53, 82, 105, 149, 184 and 218.

The purified target fragments were integrated into a donor vector by a BP reaction. The so formed entry clone facilitated then by an LR reaction the integration of the fragments in the destination vector pK7GWIWG2D(II) in an antisense-sense orientation, separated by an intron, the chloramphenicol resistance gene and a second intron to form a dsRNA hairpin construct. BP (Invitrogen Cat. No 11789-100) and LR reaction (Invitrogen Cat. No. 11791-100) were done according to the manufacturer's manual. Correct integration of the target fragments was confirmed by sequencing. The plant expression vectors comprising the Rhizoctonia solani hairpins were subsequently transformed into Agrobacterium tumefaciens.

For the two other R. solani subspecies, fragments of target genes are selected and cloned in hairpins in a similar way.

### Example 4

### Transformation of Rice with a Plant Expression Vector Comprising a Rhizoctonia solani Hairpin Sequence and Testing of the Transformed Rice Plants for Resistance to R. solani

a) Generation of Transgenic Rice Material

Genetically enhanced rice events are generated by an Agrobacterium tumefaciens based transformation strategy by inoculating embryogenic callus derived from the mature seed. The binary vector contains a plant selectable marker (NPTII). A second expression cassette encodes for the specific Rhizoctonia solani target sequence cloned as a hairpin under the control of the constitutive promoter (the CaMV 35S promoter) and the 35S terminator sequence (see Example 3 for the making of the hairpin constructs).

‘Cheniere’ (Oryza sativa L.) (Reg. no. CV-120, PI 634719, NSSL 428621.52) is a high-yielding, early maturing, semidwarf long-grain rice cultivar developed at the Rice Research Station at Crowley, L A, by the Louisiana State University Agricultural Center (LSU AgCenter) in cooperation with the USDA-ARS, the Arkansas Agricultural Experiment Station, the Mississippi Agricultural and Forestry Experiment Station, the Florida Agricultural Experiment Station, and the Texas Agricultural Experiment Station. Cheniere was officially released by the LSU AgCenter in 2002 (Crop Sci 46:1814-1815 (2006)).

Embryogenic callus of the variety is initiated from the mature seed. Calli at a predetermined stage are inoculated with the specific Agrobacterium strain (e.g. C58C₁Rif^{R} or EHA105) containing the binary vector of choice described above. The procedure follows the protocol of Hiei et al. (The Plant Journal (1994) 6(2): 271-282). The aminoglycoside antibiotic G418 (Duchefa product no. G0175) is included at all tissue culture steps post co-cultivation for transgenic plant selection, and Timentin (Ticarcillin disodium/Clavulanate potassium, Duchefa product no. T0190.0025) is used to inhibit any Agrobacterium re-growth. Transformation is confirmed by PCR and the primary transformants (generation T0) carrying both sequences, the selectable marker and the gene of interest, are transferred to soil for the generation of T1 seed material for the fungal bioassay.

Genomic PCR and/or Southern blotting is performed on leaf tissue of T1 plants to determine the homozygosity/heterozygosity of the integrated locus and the number of inserted copies of transgene. Transgene-positive plants are further analyzed by Northern blotting and/or RT-PCR to detect expression of dsRNA (hairpin transcript) and siRNA. Homozygous lines showing expression of dsRNA and/or siRNA are established and used for fungal infection studies.

b) Rice Assay

Rice calli are transformed and regenerated into shoots and whole plants as described above. The plants are transferred to a greenhouse and cultivated to reach maturity and to set seeds.

Explants (15-20 replicates each) from T1 plants (both heterozygous and homozygous integrants) are used for initial analysis of resistance to sheath blight infection. Seeds are sown in soil (3 seeds per pot) and raised at 28° C., 80% humidity and a 16 h light/8 h dark cycle. After 4 weeks the plants are used for infection assays with Rhizoctonia solani. A single sclerotial culture of Rhizoctonia solani is multiplied on potato dextrose agar (PDA) at 28° C. for 4 days and the emerging immature sclerotia are used for rice sheath inoculation. For inoculation, sheaths are opened carefully and a small piece of sclerotia is placed inside the sheath. Plants are kept at high humidity in environmental chambers (Convirons) until disease symptoms develop 4-5 days after inoculation. Disease severity is scored by the numbers and size of the lesions on the leaf sheath.

It will be appreciated by persons skilled in the art that numerous variations and/or modifications may be made to this assay without departing from the spirit or scope of this assay as generically described. Those skilled in the art will recognize, or be able to ascertain using no more than routine experimentation, many equivalents to the specific example, and such equivalents are intended to be encompassed by the present invention. The present example, therefore, is to be considered in all respects as illustrative and not restrictive.

### Example 5

### Transformation of otato with a Plant Expression Vector Comprising a Rhizoctonia solani Hairpin Sequence and Testing of the Transformed Rice Plants for Resistance to R. solani

a) Generation of Transgenic Potato Material

The example provided below demonstrates that transgenic potato plants expressing a Rhizoctonia solani-gene-specific hairpin confer resistance in the tubers when exposed to the fungus.

Stably transformed potato plants were obtained using an adapted protocol received through Julie Gilbert at the NSF Potato Genome Project (http://www.potatogenome.org/nsf5). Stem internode explants of potato ‘Line V’ (obtained from the Laboratory of Plant Breeding at PRI Wageningen, the Netherlands), which was derived from the susceptible diploid Solanum tuberosum 6487-9, were used as starting material for all transformation experiments.

In vitro derived explants were inoculated with Agrobacterium tumifaciens (e.g. C58C₁Rif^{R} or EHA105) containing the hairpin construct, and additionally an NPTII plant selection cassette. After three days co-cultivation the explants were put onto a selective medium containing 100 mg/I Kanamycin and 300 mg/I Timentin. After 6 weeks, post-transformation, the first putative shoots were removed and rooted on selective medium. Shoots originating from different explants were treated as independent events, shoots originating from the same callus were termed ‘siblings’ until their clonal status can be verified by Southerns, and nodal cuttings of a shoot were referred to as ‘clones’.

The transgenic status of the rooting shoots was checked either by GFP fluorescence or by plus/minus PCR for the target sequence and plant selectable marker. Positive shoots were then clonally propagated in tissue culture to ensure enough replicates were available for the fungal bioassay, with the first plants being available to test fourteen weeks post-transformation.

b) Potato Assay

Stem internode explants were transformed and regenerated into shoots and whole plants as described above. For the fungi infection assay, plants transformed with target 1, 13 or 21 (corresponding respectively with hairpin sequences SEQ ID NOs 8, 53 and 149) were selected. The number of independent transgenic events tested were 25, 22 and 23 respectively for target 1, 13 and 21. For each event, 10 replicates were tested (see Table 7). Controls included 10 replicates of wild type potatoes, and 10 replicates of 12 independent events of transgenic potatoes carrying the empty vector without the hairpin expression cassette (Table 7).

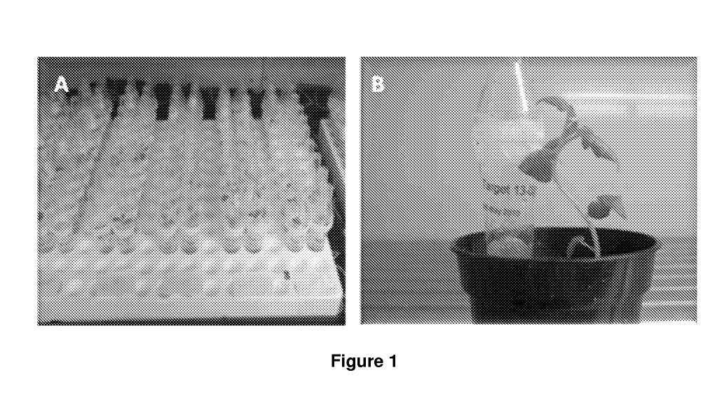

Transgenic shoots of all plantlets were allowed to grow for 3-4 weeks in individual vials in potato MS_medium (3% sucrose, 0.7% agar) (see FIG. 1). Plantlet roots were separated from the medium, washed gently with tap water to avoid any possible damage and transferred to small pots containing moist medium grade vermiculite. The plantlets were covered with polythene bags to maintain high humidity before transferring to a controlled environment chamber at 25° C. under a 16 hour photoperiod provided by cool white fluorescent tubes at 30-50 μmol m⁻² s⁻¹. Seven days after transfer, humidity was reduced gradually to the ambient level by perforating the bags and increasing the light intensity to 150-200 μmol m⁻² s⁻¹ for gradual acclimatisation. Pots were kept well watered and efforts were taken to avoid any droplets collecting on the foliage. The polythene bags were then gradually removed from the plants over a period of 7-10 days.

The Rhizoctonia soil assay was prepared by mixing the soil, naturally infected with Rhizoctonia solani, and John Innes No. 2 compost at the 1:1 ratio. The four week old plants of all plant types were transferred to Rhizoctonia infected soil in 12 cm pots. The plants were raised in the controlled environment chamber at 25° C. with a 16 hour photoperiod and a light intensity of 150-200 μmol m⁻² s⁻¹ provided by cool white fluorescent tubes until assessed for Rhizoctonia sensitivity.

The assessments of the plants were performed over a period of 78 days post transplantation into infected soil. At the end of the trial the potatoes were dug up for a comprehensive assessment.

The assessment included recordings of:
plant vigour, over 78 days: on a 0-10 scale, where 0 is no growth and 10 is the most vigorous
disease severity, at the end of the trial: on a 0-5 scale
   0=no infection
   1=brown discoloration of underground stem
   2=distinct Rhizoctonia lesions, covering <25% of underground stem circumference
   3=severe Rhizoctonia infection, covering 25-50% of underground stem circumference
   4=canker completely girdling underground stem, 51-75% of the total surface area covered with Rhizoctonia
   5=stems completely nipped off, 76-100% of total surface area covered with Rhizoctonia and/or death of the plant
incidence of infection at the end of the trial: calculated % of replicates showing infection

c) Assay Results

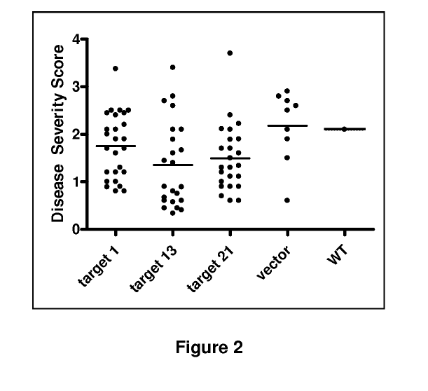

The “disease severity”, “Rhizoctonia incidence” and “plant vigour” data, were compared in the transgenic events and the wild type plants (see FIG. 2). The wild type plants showed an average score of disease severity of 2.10, calculated as averages of at least 10 clones for each line, with an incidence of 50% indicating that half of the plants were infected.

The distribution of the disease severity demonstrated that some events showed resistance to the Rhizoctonia infection. Differences between the events were explained by biological variability of both the transgenic plants and of the infectious fungi. In addition, the plants included in these assays were primary transformants, uncharacterized molecularly and could carry a wide range of copies of the T-DNA. Nevertheless, differences were observed between events and the vector control (FIG. 2).

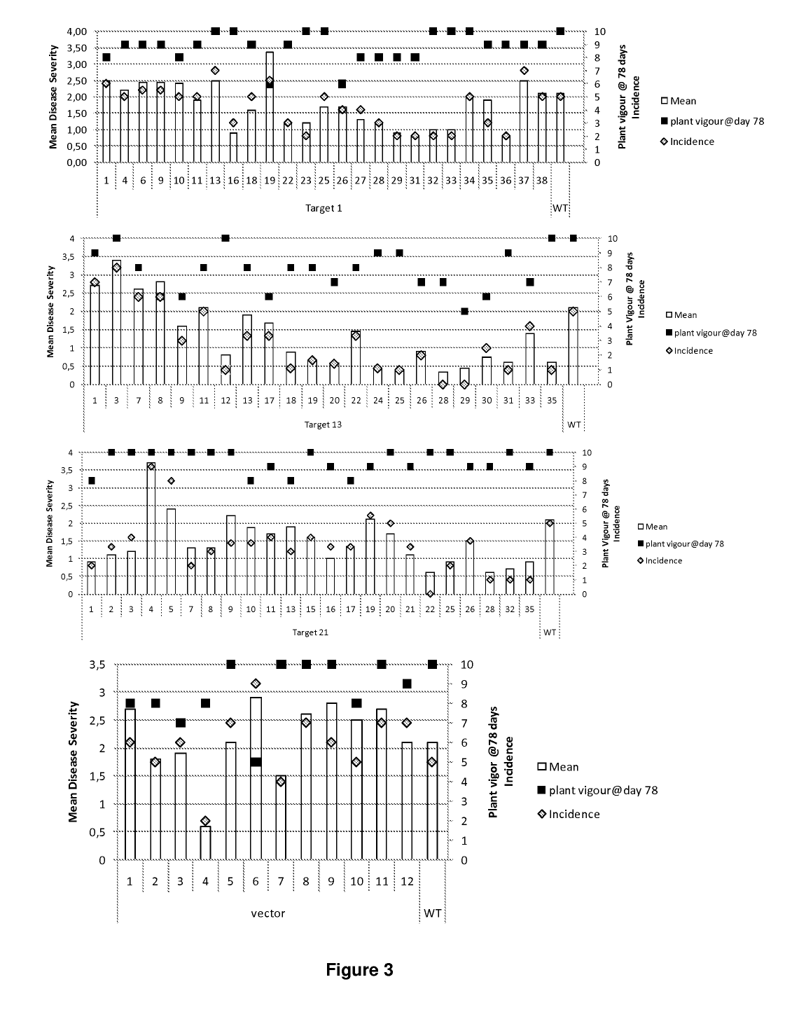

Taking into consideration the results for each event individually indicated that some events displayed low disease severity and high vigour (FIG. 3). These events were considered as “interesting” and were cultured for further molecular characterization.

It will be appreciated by persons skilled in the art that numerous variations and/or modifications may be made to this assay without departing from the spirit or scope of this assay as generically described. Those skilled in the art will recognize, or be able to ascertain using no more than routine experimentation, many equivalents to the specific example, and such equivalents are intended to be encompassed by the following claims. The present example, therefore, is to be considered in all respects as illustrative and not restrictive.

TABLE 1
Succesful primer pairs for amplification of the target sequences

| Tar- | SEQ ID NO(s) forward | SEQ ID NO(s) backward |
|---|---|---|
| get | primer(s) | primer(s) |
| 01 | 9, 10, 11 and 21 | 16, 17 and 22 |
| 06 | 31 and 33 | 41 and 42 |
| 13 | 56, 62 and 77 | 66, 73 and 78 |
| 15 | 83 and 84 | 93 and 94 |
| 16 | 107 and 110 | 122 and 125 |
| 21 | 153, 159 and 161 | 163, 164, 174 and 176 |
| 27 | 185 | 195 |
| 28 | 224, 226, 228, 231 and 232 | 238, 243, 244, 245 and 250 |

TABLE 2
Rhizoctonia solani ZG3 (anastomosis
group AG11) target sequences

| | | | | SEQ ID |
|---|---|---|---|---|
| | | SEQ ID | SEQ ID | NO(s) |
| | | NO(s) | NO(s) | of target |
| Tar- | | nucleic | amino | nucleic acid |
| get | Function | acid(s) | acid(s) | fragment(s) |
| 1 | Proteasome subunit | 1 | 2 | 7, 21 and 22 |
| 6 | Component of the | 23 | 24 | 25 |
| | spliceosome | | | |
| 13 | Helicase | 46 | 47 | 52, 77 |
| | | | | and 78 |
| 15 | Involved in intracellular | 79 | 80 | 81 |
| | transport, ESCRT pathway | | | |
| 16 | Component of the COPI | 98 | 99 | 104 |
| | vesicle coat | | | |
| 21 | Sec23, GTPase activator | 136, 138 | 137, 139 | 148 |
| | activity involved in ER | and 142 | and 143 | |
| | to Golgi transport | | | |
| 27 | δ-coatoamer | 179 | 180 | 183 |
| 28 | RNA polymerase II, 140 kd | 207, 209 | 208, 210 | 217 and |
| | subunit | and 211 | and 212 | 250 |

TABLE 3
Rhizoctonia solani ZG5 (anastomosis
group AG2-1) target sequences

| | | SEQ ID | SEQ ID |
|---|---|---|---|
| | | NO(s) | NO(s) |
| Tar- | | nucleic | amino |
| get | Function | acid(s) | acid(s) |
| 1 | Proteasome subunit | 3 | 4 |
| 6 | Component of the spliceosome | — | — |
| 13 | Helicase | 48 | 49 |
| 15 | Involved in intracellular | — | — |
| | transport, ESCRT pathway | | |
| 16 | Component of the COPI vesicle coat | 100 | 101 |
| 21 | Sec23, GTPase activator activity | 140 and | 141 and |
| | involved in ER to Golgi transport | 144 | 145 |
| 27 | δ-coatoamer | — | — |
| 28 | RNA polymerase II, 140 kd subunit | 213 | 214 |

TABLE 4
Rice infecting Rhizoctonia solani (anastomosis
group AG1-1A) target sequences

| | | SEQ ID | SEQ ID |
|---|---|---|---|
| | | NO(s) | NO(s) |
| Tar- | | nucleic | amino |
| get | Function | acid(s) | acid(s) |
| 1 | Proteasome subunit | 5 | 6 |
| 6 | Component of the spliceosome | — | — |
| 13 | Helicase | 50 | 51 |
| 15 | Involved in intracellular | — | — |
| | transport, ESCRT pathway | | |
| 16 | Component of the COPI vesicle coat | 102 | 103 |
| 21 | Sec23, GTPase activator activity | 146 | 147 |
| | involved in ER to Golgi transport | | |
| 27 | δ-coatoamer | 181 | 182 |
| 28 | RNA polymerase II, 140 kd subunit | 215 | 216 |

TABLE 5
Specific primer pairs for adding the att sites

| Tar- | SEQ ID NO(s) target specific | SEQ ID NO(s) target specific |
|---|---|---|
| get | forward primer(s) | backward primer(s) |
| 1 | 19 | 20 |
| 6 | 44 | 45 |
| 13 | 75 | 76 |
| 15 | 96 | 97 |
| 16 | 134 | 135 |
| 21 | 177 | 178 |
| 27 | 205 | 206 |
| 28 | 248 | 249 |

TABLE 6
Further embodiments of the invention

| | |
|---|---|
| a) | an amino acid sequence that, when the two sequences are optimally aligned and |
| | compared, is at least 70%, 72%, 74%, 76%, 78%, 80%, 85%, 90% or 95% identical to any |
| | of SEQ ID NOs 24, 47, 80, 99, 101 or 143 over the total length of SEQ ID NOs 24, 47, 80, |
| | 99, 101 or 143, respectively |
| b) | an amino acid sequence that, when the two sequences are optimally aligned and |
| | compared, is at least 95%, 96%, 97%, 98% or 99% identical to SEQ ID NO 2, preferably |
| | over a length of at least 50, 60, 70, 80, 90, 100 or 105 amino acids |
| c) | an amino acid sequence that, when the two sequences are optimally aligned and |
| | compared, is at least 91%, 92%, 93%, 94%, 95%, 96%, 97%, 98% or 99% identical to SEQ |
| | ID NO 4 or 6 over a length of at least 50, 60, 70, 80, 90 or 96 amino acids |
| d) | an amino acid sequence that, when the two sequences are optimally aligned and |
| | compared, is at least 85%, 86%, 87%, 88%, 89%, 90%, 91%, 92%, 93%, 94%, 95%, 96%, |
| | 97%, 98% or 99% identical to SEQ ID NO 49 or 51, preferably over a length of at least 50, |
| | 75, 100, 125, 150, 175, 200, 225, 250 or 275 amino acids |
| e) | an amino acid sequence that, when the two sequences are optimally aligned and |
| | compared, is at least 82%, 83%, 84%, 85%, 86%, 87%, 88%, 89%, 90%, 91%, 92%, 93%, |
| | 94%, 95%, 96%, 97%, 98% or 99% identical to SEQ ID NO 103 or 137, preferably over a |
| | length of at least 100, 120, 140, 160 or 177 amino acids |
| f) | an amino acid sequence that, when the two sequences are optimally aligned and |
| | compared, is at least 86%, 87%, 88%, 89%, 90%, 91%, 92%, 93%, 94%, 95%, 96%, 97%, |
| | 98% or 99% identical to SEQ ID NO 139, preferably over a length of at least 50, 75, 100, |
| | 125, 150, 175, 200, 225, 250 or 270 amino acids |
| g) | an amino acid sequence that, when the two sequences are optimally aligned and |
| | compared, is at least 86%, 87%, 88%, 89%, 90%, 91%, 92%, 93%, 94%, 95%, 96%, 97%, |
| | 98% or 99% identical to SEQ ID NO 141, preferably over a length of 50, 75, 100, 125, 150, |
| | 175, 200 or 227 amino acids |
| h) | an amino acid sequence that, when the two sequences are optimally aligned and |
| | compared, is at least 91%, 92%, 93%, 94%, 95%, 96%, 97%, 98% or 99% identical to SEQ |
| | ID NO 145, preferably over a length of at least 250, 300, 350, 400, 450, 500 or 530 amino |
| | acids |
| i) | an amino acid sequence that, when the two sequences are optimally aligned and |
| | compared, is at least 94%, 95%, 96%, 97%, 98% or 99% identical to SEQ ID NO 147, |
| | preferably over a length of at least 50, 60, 70, 80, 90, 100 or 118 amino acids |
| j) | an amino acid sequence that, when the two sequences are optimally aligned and |
| | compared, is at least 92%, 93%, 94%, 95%, 96%, 97%, 98% or 99% identical to SEQ ID |
| | NO 180 or 182, preferably over a length of at least 50, 75, 100, 125, 150, 175 or 182 |
| | amino acids |
| k) | an amino acid sequence that, when the two sequences are optimally aligned and |
| | compared, is at least 80%, 81%, 82%, 83%, 84%, 85%, 86%, 87%, 88%, 89%, 90%, 91%, |
| | 92%, 93%, 94%, 95%, 96%, 97%, 98% or 99% identical to SEQ ID NO 208, preferably over |
| | a length of at least 250, 300, 350, 400, 450, 500, 550, 600 or 624 amino acids |
| l) | an amino acid sequence that, when the two sequences are optimally aligned and |
| | compared, is at least 85%, 86%, 87%, 88%, 89%, 90%, 91%, 92%, 93%, 94%, 95%, 96%, |
| | 97%, 98% or 99% identical to SEQ ID NO 210, preferably over a length of at least 250, |
| | 300, 350, 400, 450, 500, 550 or 596 amino acids |
| m) | an amino acid sequence that, when the two sequences are optimally aligned and |
| | compared, is at least 88%, 89%, 90%, 91%, 92%, 93%, 94%, 95%, 96%, 97%, 98% or |
| | 99% identical to SEQ ID NO 212, preferably over a length of 50, 75, 100, 125, 150, 175, |
| | 200 or 217 amino acids |
| n) | an amino acid sequence that, when the two sequences are optimally aligned and |
| | compared, is at least 93%, 94%, 95%, 96%, 97%, 98% or 99% identical to SEQ ID NO 214, |
| | preferably over a length of 50, 75, 100, 125, 150, 175, 200, 225 or 255 amino acids |
| o) | an amino acid sequence that, when the two sequences are optimally aligned and |
| | compared, is at least 91%, 92%, 93%, 94%, 95%, 96%, 97%, 98% or 99% identical to SEQ |
| | ID NO 216, preferably over a length of 50, 75, 100, 120, 140 or 163 amino acids |

TABLE 7
Details of the plants included in the in planta assay

| | | Total number |
|---|---|---|
| Plants | Details | of plants |
| Non transgenic control | 10 replicates | 10 |
| Vector control | 10 replicates x 12 events | 120 |
| Target 1 | 10 replicates x 25 events | 250 |
| Target 13 | 10 replicates x 22 events | 220 |
| Target 21 | 10 replicates x 23 events | 230 |

1. A method for preventing and/or controlling pathogenic Rhizoctonia spp. infestation, comprising exposing a pathogenic Rhizoctonia to at least one dsRNA comprising annealed complementary strands, whereby ingestion of said dsRNA by said Rhizoctonia inhibits the expression of a Rhizoctonia target gene, which target gene comprises a nucleotide sequence that is complementary to one of the strands of said dsRNA.

2. The method according to claim 1, wherein at least 20% Rhizoctonia mortality or at least 20% Rhizoctonia control is obtained by silencing of an essential target gene of a pathogenic Rhizoctonia spp. by application of at least one dsRNA comprising annealed complementary strands, one of which comprises at least 21, preferably at least 22, 23 or 24 contiguous nucleotides that are complementary to a Rhizoctonia target gene essential to said Rhizoctonia spp., wherein said Rhizoctonia target gene
(i) is selected from the group of genes having a nucleotide sequence comprising any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof, or
(ii) is selected from the group of genes having a nucleotide sequence that, when the two sequences are optimally aligned and compared using the BLASTN alignment tool, is at least 75% identical to any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof, or
(iii) is selected from the group of genes having a nucleotide sequence encoding an amino acid sequence that, when the two sequences are optimally aligned and compared using the BLASTP alignment tool, is at least 70% identical to any of SEQ ID NOs 47, 49, 51, 2, 4, 6, 24, 80, 99, 101, 103, 137, 139, 141, 143, 145, 147, 180, 182, 208, 210, 212, 214 or 216,
as compared to the control Rhizoctonia spp. applied with a dsRNA targeting a non-essential gene or a gene not naturally expressed in said pathogenic Rhizoctonia spp.

3. The method according to claim 1, comprising exposing the pathogenic Rhizoctonia spp. to an agent comprising at least one dsRNA that functions upon uptake by the Rhizoctonia to inhibit the expression of a target gene within said Rhizoctonia, wherein said dsRNA comprises annealed complementary strands, one of which comprises a ribonucleotide sequence that is complementary to the sequence of said target gene, wherein said ribonucleotide sequence is transcribed from:
(i) a DNA sequence selected from the group of sequences comprising any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof, or a fragment thereof, or
(ii) a DNA sequence selected from the group of sequences having, when the two sequences are optimally aligned and compared using the BLASTN alignment tool, at least 75% identity with any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof, or
(iii) a DNA sequence selected from the group of sequences encoding an amino acid sequence that, when the two sequences are optimally aligned and compared using the BLASTP alignment tool, is at least 70% identical to any of SEQ ID NOs 47, 49, 51, 2, 4, 6, 24, 80, 99, 101, 103, 137, 139, 141, 143, 145, 147, 180, 182, 208, 210, 212, 214 or 216.

4. The method according to claim 1, for preventing and/or controlling the infection of a plant, part of a plant, reproductive plant material, seed or tuber(s) by a pathogenic Rhizoctonia spp., comprising expressing in or applying to said plant, part of a plant, reproductive plant material, seed or tuber(s) at least one dsRNA comprising annealed complementary strands, one of which comprises at least 21, preferably at least 22, 23 or 24 contiguous nucleotides that are complementary to a Rhizoctonia target gene essential to said Rhizoctonia, wherein said Rhizoctonia target gene
(i) is selected from the group of genes having a nucleotide sequence comprising any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof, or
(ii) is selected from the group of genes having a nucleotide sequence that, when the two sequences are optimally aligned and compared using the BLASTN alignment tool, is at least 75% identical to any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof, or
(iii) is selected from the group of genes having a nucleotide sequence encoding an amino acid sequence that, when the two sequences are optimally aligned and compared using the BLASTP alignment tool, is at least 70% identical to any of SEQ ID NOs 47, 49, 51, 2, 4, 6, 24, 80, 99, 101, 103, 137, 139, 141, 143, 145, 147, 180, 182, 208, 210, 212, 214 or 216.

5. The method according to claim 1, wherein the pathogenic Rhizoctonia spp. is a rice or a potato pathogenic Rhizoctonia spp.

6. The method according to claim 1, wherein the pathogenic Rhizoctonia spp. is a Rhizoctonia strain chosen from the group comprising Rhizoctonia solani spp., Rhizoctonia solani ZG3, anastomosis group AG11, Rhizoctonia solani ZGS, anastomosis group AG2-1 and the rice infecting Rhizoctonia solani, anastomosis group AG1-1A.

7. The method according to claim 1, for preventing and/or controlling the infection of a substrate or material by a Rhizoctonia spp., comprising applying to said substrate or material at least one dsRNA comprising annealed complementary strands, one of which comprises at least 21, preferably at least 22, 23 or 24 contiguous nucleotides that are complementary to a Rhizoctonia target gene essential to said Rhizoctonia, wherein said Rhizoctonia target gene
(i) is selected from the group of genes having a nucleotide sequence comprising any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof, or
(ii) is selected from the group of genes having a nucleotide sequence that, when the two sequences are optimally aligned and compared using the BLASTN alignment tool, is at least 75% identical to any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof, or
(iii) is selected from the group of genes having a nucleotide sequence encoding an amino acid sequence that, when the two sequences are optimally aligned and compared using the BLASTP alignment tool, is at least 70% identical to any of SEQ ID NOs 47, 49, 51, 2, 4, 6, 24, 80, 99, 101, 103, 137, 139, 141, 143, 145, 147, 180, 182, 208, 210, 212, 214 or 216.

8. The method according to claim 1, wherein said dsRNA is expressed by at least one prokaryotic or eukaryotic host cell or host organism.

9. The method of claim 8, wherein the prokaryotic cell is a bacterial cell chosen from the group comprising Gram positive and Gram negative cells comprising Escherichia spp. (such as for instance E. coli), Bacillus spp. (such as for instance B. thuringiensis), Rhizobium spp., Lactobacillus spp., Lactococcus spp., Pseudomonas spp. and Agrobacterium spp.

10. The method of claim 9, wherein the bacterial cell is inactivated by heat or by chemical treatment.

11. The method of claim 8, wherein the eukaryotic cell or organism is a plant cell or a plant.

12. A nucleic acid molecule selected from the group consisting of:
(i) a nucleic acid molecule which comprises at least 21, preferably at least 22, 23 or 24 contiguous nucleotides of a nucleotide sequence as represented by any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof, or
(ii) a nucleic acid molecule which comprises at least 21, preferably at least 22, 23 or 24 contiguous nucleotides of a nucleotide sequence that, when the two sequences are optimally aligned and compared using the BLASTN alignment tool, is at least 75% identical to any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof, or
(iii) a nucleic acid molecule encoding an amino acid sequence that, when the two sequences are optimally aligned and compared using the BLASTP alignment tool, is at least 70% identical to any of SEQ ID NOs 47, 49, 51, 2, 4, 6, 24, 80, 99, 101, 103, 137, 139, 141, 143, 145, 147, 180, 182, 208, 210, 212, 214 or 216.

13. An expression cassette comprising at least one regulatory sequence that directs the expression of at least one nucleic acid molecule selected from:
(i) a nucleic acid molecule of claim 12, or
(ii) a nucleic acid molecule having a first region containing a nucleotide sequence of a nucleic acid molecule of claim 12 identical to at least 21 contiguous nucleotides of a selected target gene, wherein the target gene is pathogenic Rhizoctonia gene, and a second region which is complementary to the first region, said expression cassette being capable of expressing a dsRNA.

14. A dsRNA produced from the expression of a nucleic acid molecule of claim 12.

15. A host cell comprising at least one nucleic acid molecule of claim 12.

16. A host cell according to claim 15, wherein said cell is chosen from a prokaryotic cell, including bacterial cells, or an eukaryotic cell, including yeast cells, plant cells and animal cells.

17. A composition comprising at least one dsRNA of claim 14.

18. A transgenic plant or plant cell comprising at least one nucleic acid molecule of claim 12.

19. A transgenic plant or plant cell according to claim 18, wherein the plant or plant cell is chosen from rice, potato or turfgrass.

20. A transgenic plant or plant cell according to claim 18,
(i) wherein the nucleic acid molecule(s) or the expression cassette(s) comprise(s) more than one nucleic acid molecule having a nucleotide sequence corresponding to more than one target gene, or
(ii) wherein the at least one dsRNA is produced from the expression of a nucleic acid molecule or an expression cassette that comprises more than one nucleotide sequence corresponding to more than one target gene,
wherein the more than one target gene can be a target gene from the same pathogenic Rhizoctonia strain or from different pathogenic Rhizoctonia strains.

21. A method of making a transgenic plant or plant cell which contains at least one dsRNA comprising annealed complementary strands, one of which comprises at least 21 contiguous nucleotides identical to a target gene in a pathogenic Rhizoctonia spp., said method comprising the steps of:
a) providing a nucleic acid molecule of claim 12, wherein the nucleic acid molecule is able to form at least one dsRNA once expressed in the plant; wherein the at least one nucleic acid molecule expressed from said expression cassette is able to form at least one dsRNA once expressed in the plant;
b) transforming a recipient plant or plant cell with said nucleic acid molecule or with said expression cassette;
c) producing one or more offspring of said recipient plant or plant cells; and
d) testing the offspring for expression of said dsRNA.

22. A transgenic plant or plant cell obtained by the method of claim 21.

23. A method of producing a plant that is resistant to Rhizoctonia spp. infection comprising:
(a) crossing a transgenic plant of claim 18 with another plant, and
(b) selecting Rhizoctonia-resistant progeny by analyzing for the presence of a nucleotide sequence that encodes at least one dsRNA comprising annealed complementary strands, one of which comprises at least 21 contiguous nucleotides identical to at least one sequence selected from the group consisting of any of SEQ ID NOs 46, 48, 50, 52, 1, 3, 5, 7, 21, 22, 23, 25, 77, 78, 79, 81, 98, 100, 102, 104, 136, 138, 140, 142, 144, 146, 148, 179, 181, 183, 207, 209, 211, 213, 215, 217 or 250, or the complement thereof.

24. The method of claim 23, wherein the plant is chosen from rice, potato or turfgrass.

25. Seed or tuber(s) produced from the plants obtained by the method of claim 21.

26. Hybrid seed or transgenic tuber(s) produced by crossing a first inbred plant with a second, distinct inbred plant wherein the first or second inbred plant is a transgenic plant of claim 18, and wherein the plant is chosen from rice, potato or turfgrass.

27. A method for producing hybrid seed or transgenic tuber(s) of claim 26, wherein crossing comprises the steps of:
(a) planting the seeds or tuber(s) of first and second inbred plants;
(b) cultivating the seeds or tuber(s) of said first and second inbred plants into plants that bear flowers;
(c) preventing self pollination of at least one of the first or second inbred plant;
(d) allowing cross-pollination to occur between the first and second inbred plants; and
(e) harvesting seeds or tuber(s) on at least one of the first or second inbred plants, said seeds or tuber(s) resulting from said cross-pollination;
wherein the plant is chosen from rice, potato or turfgrass.

28. A hybrid plant produced by growing the seed or tuber(s) of claim 26, wherein the plant is chosen from rice, potato or turfgrass.
